(19) United States
(12) Patent Application Publication
(10) Pub. No.: US 2006/0147491 A1
(43) Pub. Date: Jul. 6, 2006

(54) Biodegradable coating compositions including multiple layers

(76) Inventors: David M. DeWitt, Minneapolis, MN (US); Robert W. Hergenrother, Eden Prairie, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/316,787

(22) Filed: Dec. 22, 2005

Related U.S. Application Data
(60) Provisional application No. 60/641,557, filed on Jan. 5, 2005.

(51) Int. Cl. A61F 2/00

(52) U.S. Cl. 424/426

(57) ABSTRACT

The invention provides devices for treatment of a patient, wherein at least a portion of the device is provided with a biodegradable coating composed of multiple coated layers of biodegradable material. The invention further provides methods of treatment utilizing the devices.

### CROSS-REFERENCE TO RELATED APPLICATIONS

The present non-provisional Application claims the benefit of commonly owned provisional Application having Ser. No. 60/641,557, filed on Jan. 5, 2005, and entitled BIODEGRADABLE COATING COMPOSITIONS INCLUDING MULTIPLE LAYERS.

### FIELD OF THE INVENTION

The invention relates to medical devices having a biodegradable component that are useful for effectively treating a treatment site within a patient's body, for example, treatment of vascular structures and other areas within the body. More specifically, the invention relates to biodegradable coating compositions for drug delivery in association with implantable medical devices.

### BACKGROUND OF THE INVENTION

Tubular organs and structures such as blood vessels are subject to narrowing or occlusion of the lumen. Such narrowing or occlusion can be caused by a variety of traumatic or organic disorders, and symptoms can range from mild irritation and discomfort to paralysis and death. Treatment is typically site-specific and varies with the nature and extent of the occlusion.

Life threatening stenoses are most commonly associated with the cardiovascular system and are often treated using percutaneous transluminal coronary angioplasty (PTCA). This process improves the narrowed portion of the lumen by expanding the vessel's diameter at the blockage site using a balloon catheter. However, three to six months after PTCA, approximately 30% to 40% of patients experience restenosis. Restenosis is thought to be initiated by injury to the arterial wall during PTCA. Restenosis primarily results from vascular smooth muscle cell proliferation and extracellular matrix secretion at the injured site. Restenosis is also a major problem in non-coronary artery disease including the carotid, femoral, iliac, and renal arteries.

Stenosis of non-vascular tubular structures is often caused by inflammation, neoplasm and/or benign intimal hyperplasia. In the case of esophageal and intestinal strictures, the obstruction can be surgically removed and the lumen repaired by anastomosis. The smaller transluminal spaces associated with ducts and vessels can also be repaired in this fashion; however, restenosis caused by intimal hyperplasia is common. Furthermore, dehiscence is also frequently associated with anastomosis requiring additional surgery, which can result in increased tissue damage, inflammation, and scar tissue development leading to restenosis.

Much recent attention has been directed to drug eluting stents (DES) that present or release bioactive agent into their surroundings (for example, luminal walls or coronary arteries). Generally speaking, bioactive agent can be coupled to the surface of a medical device by surface modification, embedded and released from within polymer materials (matrix-type), or surrounded by and released through a carrier (reservoir-type). The polymer materials in such applications should optimally act as a biologically inert barrier and not induce further inflammation within the body. However, the molecular weight, porosity of the polymer, and the thickness of the polymer coating can contribute to adverse reactions to the medical device.

An ongoing technical challenge with present drug eluting coatings applied to devices such as stents is achieving a therapeutic concentration of a bioactive agent locally at a target site for a prescribed time within the body without producing unwanted systemic side effects. Implantation of vascular stents is a prime example of a situation where local therapy is needed utilizing bioactive agents that can also produce unwanted systemic side effects. Because the stent is placed within a flowing blood stream, during placement and upon implantation, potential unwanted systemic effects may result from undesirable quantities (for example, undesirably high quantities) of the therapeutic substance entering the blood stream. Further, if quantities of therapeutic substance are released into the blood stream as part of a “burst” effect, less of the therapeutic substance is available for actual local treatment once the stent is emplaced, resulting in potential inadequate local dosing.

Some recent work has been done to utilize degradable materials in association with stents, as well as DES. Degradable devices and degradable coatings provided on devices typically have bioactive agent physically immobilized in the polymer. The bioactive agent can be dissolved and/or dispersed throughout the polymeric material. The degradable polymeric material is often hydrolytically degraded over time through hydrolysis of labile bonds, allowing the polymer to erode into the fluid, releasing the active agent into the fluid. Generally speaking, hydrophilic polymers typically have a faster rate of erosion relative to hydrophobic polymers. Hydrophobic polymers are believed to have almost purely surface diffusion of water, resulting in erosion from the surface inwards. Hydrophilic polymers are believed to allow water to penetrate the surface of the polymer, allowing hydrolysis of labile bonds beneath the surface, which can lead to homogeneous or bulk erosion of polymer.

The goal of sustained-release systems is to maintain bioactive agent levels within a therapeutic range and ideally a constant and predictable level. In order to achieve relatively constant levels, bioactive agents should be released from a delivery system at a rate that does not change with time (so called zero-order release). Preferably, the initial dose of a bioactive agent is the therapeutic dose that is maintained by the delivery system. In many systems, however, the bioactive agent release is proportional to time (zero order release) or the square root of time (Fickian release).

In nondegradable polymeric matrix systems for bioactive agent delivery, bioactive agent is dispersed throughout a matrix and is released as it dissolves and diffuses through the matrix. A bioactive agent is released from the outer surface of the matrix first, this layer becomes depleted, and the bioactive agent that is released from further within the core of the device must then diffuse through the depleted matrix. The net result is that the release rate slows down over time.

When the polymeric matrix systems are degradable, release of the bioactive agent can also occur by diffusion (as discussed for nondegradable polymeric matrix systems), and also via degradation of the polymer. The lifetime of a biodegradable polymer in vivo can depend upon its molecular weight, crystallinity, biostability, and the degree of crosslinking. In general, the greater the molecular weight, the higher the degree of crystallinity, and the greater the biostability, the slower biodegradation will be. Accordingly, degradation times can vary widely, for example, from less than one day to several months. Thus, release kinetics become even more complex from biodegradable polymeric matrix systems. As a result of the multiple mechanisms of release of bioactive agent from a biodegradable polymeric matrix, zero-order release from these types of systems is very difficult to achieve.

### SUMMARY OF THE INVENTION

Generally, the invention provides implantable medical devices including biodegradable compositions as a coating on a surface of the device. In some aspects, the polymeric formulations of the invention biodegrade within a period that is acceptable for the desired application. In certain aspects, such as in vivo therapy, such degradation occurs in a period usually less than about one year, or less than about six months, three months, one month, fifteen days, five days, three days, or even one day, on exposure to a physiological solution with a pH between 6 and 8 having a temperature in the range of about 25° to about 37° C. In some embodiments, the polymeric formulation degrades in a period in the range of about an hour to several weeks, depending upon the desired application.

In its article aspects, the invention provides a device having a surface and a coating disposed on the surface, the coating comprising a first coated layer comprising a first biodegradable polymer, a second coated layer comprising a second biodegradable polymer, and bioactive agent, wherein the first biodegradable polymer is preferably a polyether ester copolymer, such as poly(ethylene glycol) terephthalate/polybutylene terephthalate (PEGT/PBT). In some embodiments, the device is a stent, and in particular can be a vascular stent.

The biodegradable compositions are composed of multiple layers of biodegradable polymers. Optionally, more than two coated layers can comprise the coating. The first biodegradable polymer and second biodegradable polymer have different bioactive agent release rates. In some embodiments, the second biodegradable polymer has a slower bioactive agent release rate than the first biodegradable polymer. The bioactive agent is present in at least one of the coated layers.

In some article aspects, the invention provides an implantable medical article having a bioactive agent releasing coating at a surface, the coating comprising: (a) a first coated layer comprising bioactive agent and a first biodegradable polymer, wherein the first biodegradable polymer is a copolymer of polyalkylene glycol terephthalate and an aromatic polyester; and (b) a second coated layer covering at least a portion of the first coated layer and comprising a second biodegradable polymer, wherein the first biodegradable polymer and the second biodegradable polymer are different, and wherein the second biodegradable polymer is selected to have a slower bioactive agent release rate relative to the first biodegradable polymer.

In other article aspects, the invention provides an implantable medical article having a bioactive agent releasing coating at a surface, the coating comprising: (a) a first coated layer comprising bioactive agent and a second biodegradable polymer; and (b) an outer coated layer comprising a polyetherester copolymer that is a copolymer of polyalkylene glycol terephthalate and an aromatic polyester, wherein the polyetherester copolymer and the second biodegradable polymer are different, and wherein the second biodegradable polymer is selected to have a slower bioactive agent release rate relative to the polyetherester copolymer.

In addition to polyether ester copolymers, other polymers containing ester linkages that are suitable first biodegradable polymers include terephthalate esters with phosphorus-containing linkages, and segmented copolymers with differing ester linkages. Other suitable first biodegradable polymers include polycarbonate-containing random copolymers. The second biodegradable polymer is selected to modify the bioactive agent release rate from the biodegradable composition, to achieve a controlled release rate.

In some aspects, the biodegradable composition comprises a coating on a surface, such as a surface of an implantable device. A “coating” as described herein can include one or more “coated layers,” each coated layer including one or more coating components. In some cases, the coating includes a first coated layer composed of a first biodegradable polymer and a second coated layer composed of a second biodegradable polymer. Bioactive agent is present in one or more of the coated layers. When more than one coated layer is applied to the surface of a device, it is typically applied successively. For example, a coated layer is typically formed by dipping, spraying, or brushing a coating material on a device to form a layer, and then drying the coated layer. The process can be repeated to provide a coating having multiple coated layers, wherein at least one layer includes bioactive agent. Typically (but not always), at least the coated layer located nearest the device surface includes bioactive agent. For example, in some embodiments, the first coated layer can comprise a first biodegradable polymer and bioactive agent, and a second coated layer can comprise a second biodegradable polymer alone (without bioactive agent).

For purposes of discussion, the coating will be described as containing a “first” coated layer, a “second” coated layer, and so on. Designation of the coated layers in this manner is meant to distinguish the chemical composition of the coated layers and does not necessarily ascribe a particular location of the coated layer in relation to the device surface and/or the other coated layers. Typically, but not necessarily, the first coated layer is located nearest the device surface. The second coated layer is typically, but not necessarily, applied over the first coated layer, and thus located at the outermost surface of the device for a two-layer coating, and so on. Describing the coated layers in this sequential fashion is utilized for purposes of illustrating the inventive concepts only, and such discussion is not intended to limit the composition of the coated layers in any particular order. For example, the order of application of the coated layers can be modified such that the first biodegradable polymer is utilized in an outermost coated layer, while the second biodegradable polymer is utilized in a coated layer at the device surface.

In some aspects, more than two coated layers can be present. Such other layers can be the same or different than the first coated layer and/or second coated layer. The suitability of the coating for use with a particular medical article, and in turn, the suitability of the application technique, can be evaluated by those skilled in the art, given the present description.

In its method aspects, the invention provides methods of making a device for controlled release of a bioactive agent, the method comprising steps of providing a device having a surface, providing a multiple layer biodegradable coating composition to the surface, the coating composed of a first coated layer, a second coated layer, and bioactive agent. The first coated layer comprises a first biodegradable polymer, and the second coated layer comprises a second biodegradable polymer. The bioactive agent is present in at least one of the coated layers. More than two coated layers can compose the coating, if desired. Preferably, no treatment steps (such as heating, application of pressure, and the like) are required between application of the individual coated layers.

In further aspects, the invention provides methods for delivery of bioactive agent to a patient in a controlled manner, the method comprising steps of providing a device to a patient, the device having a surface and a biodegradable coating composition disposed on the surface, the biodegradable coating composition comprising a first coated layer, a second coated layer, and bioactive agent. In some aspects, the method includes a step of maintaining the device in the patient for a selected period of time, during which time the bioactive agent is released from the coating composition in a controlled and predictable manner.

In a more specific aspect, the invention provides devices and methods for providing treatment (for example, of vascular structures), wherein the devices include at least a component that is biodegradable. In preferred aspects, any portions of the device that remain in the body (are not degraded and/or resorbed) do not cause significant adverse foreign body response.

Preferred compositions and methods according to the invention provide the ability to control the release rate of bioactive agent from the device surface over time. In some aspects, such control is provided by selecting the second polymer and adjusting the relative amounts of the first polymer and second polymer to achieve the desired release profile of the bioactive agent. The rate of bioactive agent release from the first polymer and the second polymer are different. Similarly, when the coating comprises more than two coated layers, controlled release of bioactive agent can be accomplished by selection of the second, third, and (optional) subsequent polymers. The relative amounts of such subsequent polymers as well as the relative position of each coated polymer layer within the coating can also be controlled. The rate of release of the individual polymers comprising the coating are preferably different.

In preferred aspects, the inventive biodegradable compositions are selected to provide a controlled release profile of bioactive agent from the biodegradable coatings. The release profile is the cumulative mass of bioactive agent released versus time. The time profile of the release of bioactive agent, including immediate release and subsequent, sustained release can be predictably controlled utilizing the inventive compositions and methods. In some aspects of the invention, the initial release of bioactive agent is controlled, thereby permitting more of the bioactive agent to remain available at later times for a more extended release duration. The shape of the release profile after an initial release can be controlled to be linear, logarithmic, or some more complex shape, depending upon the composition of the coated layers of the coating and bioactive agent(s) in the coating. In some embodiments, additives can be included in the biodegradable composition to further control the release rate. In preferred aspects, the inventive biodegradable compositions maintain bioactive agent levels within a therapeutic range and ideally a relatively constant level.

Surprisingly, some embodiments of the invention provide devices and methods of reproducibly releasing bioactive agent in a linear manner over extended periods of time. As described herein, in vitro elution assays of preferred embodiments of the invention show surprisingly controllable release of bioactive agent over time. In preferred embodiments, coating compositions having varying formulations (in terms of polymer ratios) can provide substantially linear release rates of bioactive agent. Based upon the in vitro data presented herein, it is expected that in vivo release rates will provide reproducible release rates in a linear manner over an extended period of time. Thus the invention can provide controlled release of bioactive agent to an implantation site that can be adjusted to accommodate desired treatment duration and dosage. Because the invention provides local delivery of one or more bioactive agents to an implantation site, the invention also preferably avoids toxic levels of bioactive agents that can be required during systemic treatment.

The inventive biodegradable compositions can find particular application when the bioactive agent comprises a relatively small molecule. In preferred aspects, the inventive concepts provide methods to allow controlled release of small molecules achievable in a therapeutically effective manner from biodegradable coatings provided on implantable device surfaces. Small molecules are typically released from biodegradable polymeric compositions via two routes, namely, diffusion through the polymeric material and degradation of the polymer material. Thus, it can be particularly difficult to control release of such molecules, especially if one wishes to avoid or minimize a relatively fast “burst” release during the initial time period after implantation of the device. The inventive biodegradable compositions can provide improved control over release of such small molecules, for example, by modulating the initial release of the bioactive agent from the biodegradable composition. Typically, small molecule bioactive agents have a molecular weight that in general is less than about 1500.

Some illustrative bioactive agents include smaller molecules having anti-proliferative effects (such as actinomycin D, paclitaxel, taxane, and the like), anti-inflammatory agents (such as dexamethasone, prednisolone, tranilast, and the like), immunosuppressive agents (such as cyclosporine, CD-34 antibody, everolimus, mycophenolic acid, sirolimus, tacrolimus, and the like), smaller molecule antibiotics, and the like. Suitable bioactive agents have been described, for example, a comprehensive listing of bioactive agents and therapeutic compounds can be found in The Merck Index, Thirteenth Edition, Merck & Co. (2001). One of skill in the art, using the guidance of the present description, can readily select bioactive agents that are suitable to be eluted from the polymeric matrices of the invention.

In use, an implantable medical device is provided with a biodegradable coating and positioned within the body at a treatment site. In one such application, a stent is placed into a body vessel after a procedure, such as angioplasty. The stent is left in position, and the biodegradable coating is allowed to degrade. Upon placement of the stent, and thus exposure of the biodegradable coating to physiological fluids, bioactive agent is released from the stent. Typically, an initial release of the bioactive agent is observed, and over time a sustained release of the bioactive agent is observed. As the biodegradable coating degrades, bioactive agent continues to be released in a controlled manner, thereby providing a therapeutically effective amount of the bioactive agent over a treatment course to the treatment site.

These and other aspects and advantages will now be described in more detail.

### BRIEF DESCRIPTION OF THE DRAWINGS

The accompanying drawings, which are incorporated in and constitute a part of this specification, illustrate several aspects of the invention and together with the description of the preferred embodiments, serve to explain the principles of the invention. A brief description of the drawings is as follows:

FIG. 1 is a graph illustrating elution profiles for coatings containing a single coated layer, versus coatings in accordance with some aspects of the invention.

FIG. 2 is a graph illustrating elution profiles for multiple layer coatings in accordance with some aspects of the invention.

FIG. 3 is a graph illustrating elution profiles for coatings containing a single coated layer, versus coatings in accordance with some aspects of the invention.

FIG. 4 is a graph illustrating elution profiles for multiple layer coatings in accordance with some aspects of the invention.

FIG. 5 is a graph illustrating elution profiles for a coating containing a single coated layer, versus a coating in accordance with some aspects of the invention.

FIG. 6 is a graph illustrating elution profiles for a coating containing a single coated layer, versus a coating in accordance with some aspects of the invention.

FIG. 7 is a graph illustrating elution profiles for multiple layer coatings in accordance with some aspects of the invention, wherein time (T, in days) is represented on the X-axis, and percent bioactive agent eluted (%) is represented on the Y-axis.

FIG. 8 is an optical image of a surface of a coated device in accordance with some aspects of the invention, shown at 100× magnification.

FIG. 9 is an optical image of a surface of a coated device in accordance with some aspects of the invention, shown at 100× magnification.

FIG. 10 is a Scanning Electron Microscope (SEM) image of a surface of a coated device in accordance with some aspects of the invention.

FIG. 11 is a Scanning Electron Microscope (SEM) image of a surface of a coated device in accordance with some aspects of the invention.

FIG. 12 is a graph illustrating elution profiles for multiple layer coatings in accordance with some aspects of the invention, wherein time (T, in days) is represented on the X-axis, and percent bioactive agent eluted (%) is represented on the Y-axis.

FIG. 13 is an optical image of a surface of a coated device in accordance with some aspects of the invention, shown at 100× magnification.

FIG. 14 is an optical image of a surface of a coated device in accordance with some aspects of the invention, shown at 100× magnification.

FIG. 15 is a Scanning Electron Microscope (SEM) image of a surface of a coated device in accordance with some aspects of the invention.

FIG. 16 is a Scanning Electron Microscope (SEM) image of a surface of a coated device in accordance with some aspects of the invention.

FIG. 17 is a graph illustrating elution profiles for multiple layer coatings in accordance with some aspects of the invention, wherein time (T, in days) is represented on the X-axis, and percent bioactive agent eluted (%) is represented on the Y-axis.

FIG. 18 is an optical image of a surface of a coated device in accordance with some aspects of the invention, shown at 100× magnification.

FIG. 19 is an optical image of a surface of a coated device in accordance with some aspects of the invention, shown at 100× magnification.

FIG. 20 is a Scanning Electron Microscope (SEM) image of a surface of a coated device in accordance with some aspects of the invention.

FIG. 21 is a Scanning Electron Microscope (SEM) image of a surface of a coated device in accordance with some aspects of the invention.

FIG. 22 is a graph illustrating elution profiles for multiple layer coatings in accordance with some aspects of the invention, wherein time (T, in days) is represented on the X-axis, and percent bioactive agent eluted (%) is represented on the Y-axis.

FIG. 23 is an optical image of a surface of a coated device in accordance with some aspects of the invention, shown at 100× magnification.

FIG. 24 is an optical image of a surface of a coated device in accordance with some aspects of the invention, shown at 100× magnification.

FIG. 25 is a Scanning Electron Microscope (SEM) image of a surface of a coated device in accordance with some aspects of the invention.

FIG. 26 is a Scanning Electron Microscope (SEM) image of a surface of a coated device in accordance with some aspects of the invention.

### DETAILED DESCRIPTION OF THE INVENTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

The invention is directed to medical devices provided with a biodegradable material in the form of a coating. At least a portion of the device is coated with the biodegradable material, and this portion is broken down gradually by the body after implantation. In some embodiments, the biodegradable composition can be bioabsorbable in addition to being biodegradable. According to these embodiments, the biodegradable composition is resorbed by the body. It is not required that the component is resorbed by the body; in some embodiments, the biodegradable composition is broken down into a plurality of portions that are not completely resorbed by the body.

The present invention is directed to methods and apparatuses for effectively treating a treatment site within a patient's body, and in particular for treating vascular sites. According to preferred embodiments of the invention, stents are provided that can provide treatment to a site within the body for a desired period of time, after which at least a portion of the stent (such as the coating) degrades. The inventive methods and apparatuses can be utilized to deliver bioactive agent to a treatment site in a controlled manner. Such methods and apparatuses in accordance with the present invention can advantageously be used to provide flexibility in treatment duration, as well as type of bioactive agent delivered to the treatment site. In particular, the present invention has been developed for controllably providing one or more bioactive agents to a treatment site within the body for a desired treatment course.

As used herein, “controlled release” refers to release of a compound (for example, a bioactive agent) into a patient's body at a desired dosage (including dosage rate and total dosage) and duration of treatment.

The term “implantation site” refers to the site within a patient's body at which the implantable device is placed according to the invention. In turn, a “treatment site” includes the implantation site as well as the area of the body that is to receive treatment directly or indirectly from a device component. For example, bioactive agent can migrate from the implantation site to areas surrounding the device itself, thereby treating a larger area than simply the implantation site.

Bioactive agent is released from the inventive coatings over time, and this relationship can be plotted to establish a release profile (cumulative mass of bioactive agent released versus time). Typically, the bioactive agent release profile can be considered to include an initial release of the bioactive agent, and a release of the bioactive agent over time, and the distinction between these two can often be simply the amount of time. The initial release is that amount of bioactive agent released shortly after the device is implanted, and the release of bioactive agent over time includes a longer period of time (for example, the lifespan of the biodegradable composition).

In some cases, the initial release can be characterized as a “burst” release. For coatings that provide a “burst release” of bioactive agent, an initial release of a significant amount of bioactive agent is observed within a relatively short period of time after an implantable device is provided within a patient. A typical burst release is a much higher release in a relatively short amount of time (for example, more than 30% of the amount of bioactive agent contained in the coating within the first 24 hours after implantation). In contrast, coatings can provide substantially linear release of bioactive agent, wherein the initial release of bioactive agent does not comprise a significantly different slope or shape than the overall release profile. Put another way, a burst release can be characterized as an initial release that differs in magnitude of bioactive agent released as compared to release of bioactive agent over time (that is, a significant amount is released during the initial period).

The significance of a burst release can also be considered in relation to the particular polymeric material that contains the bioactive agent. For example, for a biodegradable polymer having a half-weight degradation time of four weeks, a significant burst release can be considered to be more than about 30% of the bioactive agent contained in the coating that is released within the first 24-hour period. For a biodegradable polymer having a half-weight degradation time of more than four weeks, a longer burst time period can be considered significant for the same amount of bioactive agent. For example, the half-weight degradation time of poly(D,L-lactide) (PLA) is approximately 155 days (depending upon molecular weight of the polymer) compared to 30 days for poly(D,L-lactide-co-glycolide) (PLGA). Thus, a longer time period would be considered therapeutically relevant for the burst release from PLA compared to PLGA.

In accordance with some aspects of the invention, the shape of the bioactive agent release curve can be modulated by controlling one or more characteristics of the coating, such as the chemical composition of the coated layers that make up the coating, the relative position of the coated layers comprising the coating, and/or the relative amounts of the individual polymers comprising the coating (such as the coating weight of the individual coated layers). In accordance with the invention, the time profile of the release of bioactive agent can be modulated to provide any desired shape, including immediate release where the drug elutes all at once (much like a step function) to an extremely slow, linear (i.e., zero order) release, where the drug is evenly released over many months or years. Depending on the drug and the condition being treated, a variety of release profiles can be achieved. The objective of creating coatings with multiple coated layers of polymers is to be able to attain the broad range of release profiles that lie between a step function and a low-slope, zero-order release. Preferably, the relative position of each layer of the biodegradable composition is selected to provide the desired release profile. In addition, or alternatively, the composition of the second layer (and subsequent layer(s), if included) can be selected to provide the desired release profile. By controlling the release profiles as described herein, significant improvements can be made to the efficacy of treatment with bioactive agent.

The desired release profile of the bioactive agent can depend upon such factors as the particular bioactive agent selected, the number of individual bioactive agents to be provided to the implantation site, the therapeutic effect to be achieved, the duration of the implant in the body, and other factors known to those skilled in the art.

Surprisingly, it has been discovered that the layer composition of a multiple layer biodegradable coating can be manipulated to provide significant differences in elution rate profiles. For example, it has been discovered that inclusion of a polyether ester copolymer in a topcoat layer (the layer furtherest from the device surface, and in contact with the biological environment) can increase an initial release rate as compared to similar coated compositions that do not include such a topcoat. In some instances, inclusion of a topcoat of PEGT/PBT can increase an initial release phase as compared to coated compositions that do not include a PEGT/PBT topcoat, but are otherwise compositionally equivalent. In other aspects, it has been discovered that various polymers and copolymers can be provided in a multiple layer format to provide enhanced control over release profiles. Some illustrative release profiles are shown in the Examples herein.

The inventive multiple layer coatings described herein are designed to control (such as, for example, by limiting or even eliminating) the initial burst of bioactive agent from the coating. The bioactive agent still remaining in the coating after the burst release is then released to the site of action over a longer time period. The shape of the release profile (percentage of bioactive agent released versus time) after the initial release phase can be controlled to be linear or logarithmic or some more complex shape, again depending on the composition of the layers of polymers and bioactive agent in the coating.

As used herein, a treatment course is a period of time during which bioactive agent is delivered to a patient. The duration of the treatment course is typically determined by the physician, based upon such factors as condition to be treated, the age and condition of the patient, the normal reaction time of the body to the procedure necessitating stent implantation (such as angioplasty), and the like. Typically, a treatment course will span from hours to days to weeks or even months. For example, a typical treatment course for minimizing risk of restenosis upon implantation of a stent is approximately 4 or more weeks.

The in vivo release of a bioactive agent can be approximated by observing the in vitro release of the bioactive agent. For example, an implantable device can be fabricated to include a biodegradable coating containing a bioactive agent. The coated implantable device can then be placed in an appropriate solution (for example, a buffer solution such as phosphate buffered saline or Tween acetate buffer) for a period of time. During incubation of the device, the solution can be periodically monitored to determine the in vitro release rate of the bioactive agent into the solution. The stent is removed from the solution and placed in fresh buffer solution in a new vial at periodic sampling times. Concentration of bioactive agent at each sampling time can be determined in the spent buffer by spectroscopy using the characteristic wavelength for each bioactive agent. The concentration can be converted to a mass of bioactive agent released from the coating using molar absorptivities. The cumulative mass of the released bioactive agent can be calculated by adding the individual sample mass after each removal. The release profile can be obtained by plotting the cumulative mass of released bioactive agent as a function of time. From this determined in vitro release rate, the in vivo release rate can be approximated using known techniques.

According to the invention, implantable devices include a biodegradable composition that is composed of multiple layers including a first polymer layer and a second polymer layer. The biodegradable composition further includes bioactive agent for treatment of a treatment site. Bioactive agent can be included in one or more of the coated layers. In preferred aspects, the invention provides devices and methods for providing controlled release of the bioactive agent to the treatment site.

In some aspects, the inventive biodegradable compositions can exhibit controlled release characteristics, in contrast to a bolus type administration (which includes an initial burst release of bioactive agent) in which a substantial amount of the bioactive agent is made biologically available at one time. For example, in some embodiments, upon contact with body fluids including blood, spinal fluid, lymph, or the like, the biodegradable compositions (formulated as provided herein) can permit a desired amount of initial release of bioactive agent, and subsequently provide a sustained, predictable delivery of the bioactive agent over time. This release can result in prolonged delivery of therapeutically effective amounts of any incorporated bioactive agent. Sustained release will vary in certain embodiments as described in more detail herein.

The phrase “therapeutically effective amount” is an art-recognized term. In some aspects, the term refers to an amount of the bioactive agent that, when incorporated into a biodegradable composition of the invention, produces some desired effect at a reasonable benefit/risk ratio applicable to any medical treatment. In some aspects, the term refers to that amount necessary or sufficient to eliminate or reduce risk of restenosis. The therapeutically effective amount can vary depending upon such factors as the condition being treated, the particular bioactive agent(s) being administered, the size of the patient, the severity of the condition, and the like. In preferred aspects, the therapeutically effective amount takes into account the amount of bioactive agent released from the biodegradable composition during any selected time period, particularly the time period during implantation and immediately after the device is emplaced (the initial release). Thus, the therapeutically effective amount also applies to the initial release of bioactive agent from the biodegradable composition. By controlling the initial release from the biodegradable composition, preferred embodiments can reduce or eliminate potentially undesirably high amounts of drug release during early stages after implantation. One of ordinary skill in the art can empirically determine the effective amount of a particular bioactive agent without necessitating undue experimentation.

Preferred aspects of the invention can thus provide one or more advantages, including the ability to provide sustained bioactive agent delivery that can maintain the bioactive agent concentration within a therapeutic window for a prolonged period of time and improve bioactive agent efficacy. Local delivery can reduce drug dosage, toxicity effects, and other side effects that are typically associated with administration of therapeutics.

According to the present invention, a device has been developed that can be used to treat any implantation site within the body in which it is desirable to provide a device having a coating that degrades (at least in part) during use. In some embodiments, the device is preferably used to treat an implantation site within the body in which it is desirable to restore and maintain patency of the implantation site while permitting function of the implantation site. For example, in vascular applications, the device can restore and maintain patency of the vascular site treated with the device, thus permitting continued blood flow through the treatment site. The inventive device further provides controlled release of one or more bioactive agents. According to this aspect of the invention, the device can provide controlled release of the bioactive agent to a treatment site within the body. As described herein, controlled release at the treatment site can mean control both in dosage rate and total dosage.

To facilitate the discussion of the invention, use of the invention to treat a vascular site will be addressed. Vascular treatment is selected because the features of the invention, particularly relating to controllable drug delivery capabilities can be clearly presented. Further, the ability to provide controlled and predictable delivery of a bioactive agent that can provide superior qualities while reducing risks to the patient can be a significant advance in the field. Emphasis is given to treatment of a vascular site with a stent; however, other devices such as vascular filters (for example, emboli filters) can also utilize the concepts of the invention.

It is understood that the device and methods disclosed are applicable to any treatment needs, for example, ophthalmic devices, orthopedic appliances or bone cement for repairing injuries to bone or connective tissue (for example, bone screws and other fixative devices that can be utilized to maintain relative position and stability to bones during a healing process, including, but not limited to, connective devices such as ties, tethers, and the like), coatings for degradable or nondegradable fabrics or paper substrates, scaffolds for tissue engineering, and the like.

In some embodiments, the biodegradable composition can include additional layers, for example, between the first and second layers, and/or at the outermost layer of the coated device (thus the tissue-contacting surface), while in other embodiments, the biodegradable compositions are composed of the layers described in detail herein.

In some aspects, the inventive biodegradable compositions are utilized to provide a coating composed of a first coated layer comprising a first biodegradable polymer, a second coated layer comprising a second biodegradable polymer, and a bioactive agent. The first biodegradable polymer is preferably a polyether ester copolymer, such as PEGT/PBT. Other polymers containing ester linkages that are suitable first biodegradable polymers include terephthalate esters with phosphorus-containing linkages, and segmented copolymers with differing ester linkages. In still further aspects, the first biodegradable polymer can comprise a polycarbonate-containing random copolymer. These aspects will now be described in more detail.

As used herein, the term “aliphatic” refers to a linear, branched, and/or cyclic alkane, alkene, or alkyne. Preferred aliphatic groups in polymeric materials that include phosphoester linkages are linear or branched alkanes having 1 to 10 carbon atoms, or linear alkane groups having 1 to 7 carbon atoms. As used herein, the term “aromatic” refers to an unsaturated cyclic carbon-containing compound with 4n+2π electrons.

As used herein, the term “heterocyclic” refers to a saturated or unsaturated ring compound having one or more atoms other than carbon in the ring, for example, nitrogen, oxygen or sulfur.

Generally speaking, the polyetherester copolymers are amphiphilic block copolymers that include hydrophilic (for example, a polyalkylene glycol, such as polyethylene glycol) and hydrophobic blocks (for example, polyethylene terephthalate).

In one embodiment, the polyetherester copolymer comprises a first component that is a polyalkylene glycol, and a second component, which is a polyester, formed as the reaction product from an alkylene glycol having from 2 to 8 carbon atoms and a dicarboxylic acid. The polyalkylene glycol, in one embodiment, is selected from the group consisting of polyethylene glycol, polypropylene glycol, and polybutylene glycol. In one embodiment, the polyalkylene glycol is polyethylene glycol.

In another embodiment, the polyester is selected from the group consisting of polyethylene terephthalate, polypropylene terephthalate, and polybutylene terephthalate. In a preferred embodiment, the polyester is polybutylene terephthalate.

In one preferred embodiment, the copolymer is a polyethylene glycol/polybutylene terephthalate block copolymer (referred to herein interchangeably as PEGT/PBT or PEG/PBT copolymer).

In another embodiment, the polyester has the following recurring structural formula I: 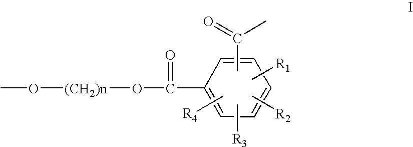
wherein n is from 2 to 8, and each of R₁, R₂, R₃, and R₄ is hydrogen, halogen (such as chlorine, iodine, bromine), nitro-, or alkoxy, and each of R₁, R₂, R₃ and R₄ is the same or different. Preferably, each of R₁, R₂, R₃, and R₄ is hydrogen. Alternatively, the polyester is derived from a binuclear aromatic diacid having the following structural formula II: 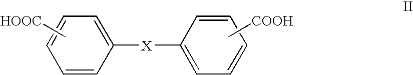
wherein X is —O—, —SO₂—, or —CH₂—.

In a preferred embodiment, the copolymer is a segmented thermoplastic biodegradable polymer comprising a plurality of recurring units of the first component and units of the second component. The first component comprises about 30 weight percent to about 99 weight percent (based upon the weight of the copolymer) of units of the formula III:
—OLO—CO—R—CO— III
wherein O represents oxygen, C represents carbon, L is a divalent organic radical remaining after removal of terminal hydroxyl groups from a poly(oxyalkylene)glycol, and R is a substituted or unsubstituted divalent radical remaining after removal of carboxyl groups from a dicarboxylic acid.

The second component is present in an amount of about 1 weight percent to about 70 weight percent (based upon the weight of the copolymer), and is comprised of units of the formula IV:
—OEO—CO—R—CO— IV
wherein E is an organic radical selected from the group consisting of a substituted or unsubstituted alkylene radical having from 2 to 8 carbon atoms, and a substituted or unsubstituted ether moiety. R is as described above in Formula III.

The poly(oxyalkylene)glycol, in one embodiment, can be selected from the group consisting of poly(oxyethylene)glycol, poly(oxypropylene)glycol, poly(oxybutylene)glycol, and combinations thereof. Preferably, the poly(oxyalkylene)glycol is poly(oxyethylene)glycol.

The poly(oxyethylene)glycol can have a molecular weight in the range of about 200 to about 20,000, or about 200 to about 10,000. The precise molecular weight of the poly(oxyethylene)glycol is dependent upon a variety of factors, including the type of bioactive agent incorporated into the biodegradable composition.

In one embodiment, E is a radical selected from the group consisting of a substituted or unsubstituted alkylene radical having from 2 to 8 carbon atoms, preferably having from 2 to 4 carbon atoms. Preferably, the second component is selected from the group consisting of polyethylene terephthalate, polypropylene terephthalate, and polybutylene terephthalate. In one embodiment, the second component is polybutylene terephthalate.

In a preferred embodiment, the copolymer is a polyethylene glycol/polybutylene terephthalate copolymer.

In one embodiment, the polyethylene glycol/polybutylene terephthalate copolymer can be synthesized from a mixture of dimethylterephthalate, butanediol (in excess), polyethylene glycol, an antioxidant, and catalyst. The mixture is placed in a reaction vessel and heated to about 180° C., and methanol is distilled as transesterification occurs. During the transesterification, the ester bond with methyl is replaced with an ester bond with butylene and/or the polyethylene glycol. After transesterification, the temperature is raised slowly to about 245° C., and a vacuum (finally less than 0.1 mbar) is achieved. The excess butanediol is distilled and a prepolymer of butanediol terephthalate condenses with the polyethylene glycol to form a polyethylene glycol/polybutylene terephthalate copolymer. A terephthalate moiety connects the polyethylene glycol units to the polybutylene terephthalate units of the copolymer, and this copolymer is sometimes hereinafter referred to as a polyethylene glycol terephthalate/polybutylene terephthalate copolymer (also referred to as PEGT/PBT or PEG/PBT copolymer). Alternatively, the polyethylene glycol is present as free polyethylene glycol that is mixed with PEGT/PBT copolymer. In another alternative, polyalkylene glycol/polyester copolymers can be prepared as described in U.S. Pat. No. 3,908,201.

The above discussion of preferred copolymers is not intended to limit the invention to the specific copolymers discussed, or to any particular synthesis means thereof.

The biodegradable composition can be formulated to provide desired degradation rates. Degradation of the biodegradable composition occurs by hydrolysis of the ester linkages, and/or oxidation of ether groups. Further, when the biodegradable composition includes a bioactive agent, the formulation of the biodegradable composition can be adjusted to control the rate of diffusion of the bioactive agent from the polymer.

In some embodiments, the degradation rate of PEGT/PBT copolymer can be controlled in two general manners. For example, the degradation rate can be increased by including hydrophilic antioxidants in the polymeric material. In addition, or alternatively, the degradation rate can be increased by partially replacing the aromatic groups with aliphatic groups. For example, the more hydrophobic aromatic groups, such as terephthalate groups, can be replaced with less hydrophobic aliphatic groups, such as diacid groups (for example, succinate). In another example, more hydrophobic butylene groups can be at least partially replaced with less hydrophobic groups, such as dioxyethylene. The degree of replacement can be determined to provide a selected effect on degradation rate.

In accordance with the invention, an increased degradation of the polyetherester copolymer is not accompanied by a significant, deleterious increase in acid formation. Degradation of the copolymer takes place by hydrolysis of ester linkages and oxidation of ether groups, which can generate a certain amount of acid. However, the levels of acid generated during degradation are, in one aspect, lesser than the levels generated by other known degradable polymers (such as PLA), and in another aspect, are not deleterious to tissues and/or bioactive agent. The acidity of the degradation environment can impact the stability of bioactive agents in that environment. Optionally, hydrophilic antioxidants can be included in the polymer material. Such hydrophilic antioxidants will be described in more detail elsewhere herein and can be particularly desirable when the biodegradable composition includes peptide or protein molecules. According to this aspect of the invention, when the protein or peptide molecule is released from the biodegradable composition upon degradation thereof, the protein is not denatured by acid degradation products. This can provide significant advantages over degradable polymers that include PLA or PLGA, where degradation increases acidity of the polymeric environment. These aspects of the invention will be described in more detail with respect to embodiments of the invention where bioactive agents are released from the biodegradable composition.

In some embodiments of the invention, the polymeric material comprises a biodegradable terephthalate copolymer that includes a phosphorus-containing linkage. Polymers having phosphoester linkages, called poly(phosphates), poly(phosphonates) and poly(phosphites), are known. See, for example, Penczek et al., Handbook of Polymer Synthesis, Chapter 17: “Phosphorus-Containing Polymers,” 1077-1132 (Hans R. Kricheldorf ed., 1992), as well as U.S. Pat. Nos. 6,153,212, 6,485,737, 6,322,797, 6,600,010, 6,419,709. The respective structures of each of these three classes of compounds, each having a different side chain connected to the phosphorus atom, is as follows:

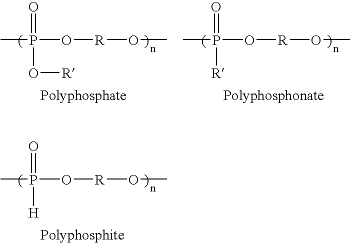

The versatility of these polymers is related to the versatility of the phosphorus atom, which is known for a multiplicity of reactions. Its bonding can involve the 3p orbitals or various 3s-3p hybrids; spd hybrids are also possible because of the accessible d orbitals. Thus, the physicochemical properties of the poly(phosphoesters) can be readily changed by varying either the R or R′ group. The biodegradability of the polymeric material according to these embodiments is related to the physiologically labile phosphoester bond in the backbone of the polymer. By manipulating the backbone or the side chain, wide ranges of biodegradation rates are attainable.

An additional feature of the poly(phosphoesters) is the availability of functional side groups. Because phosphorus can be pentavalent, bioactive agents (such as drugs) can be chemically linked to the polymer. For example, bioactive agents with carboxyl groups can be coupled to the phosphorus via an ester bond, which is hydrolyzable. The P group in the backbone also lowers the glass transition temperature (Tg) of the polymer and, importantly, confers solubility in common organic solvents, which can be desirable for characterization and processing of the polymer.

In one embodiment, the terephthalate polyester includes a phosphoester linkage that is a phosphite. Suitable terephthalate polyester-polyphosphite copolymers are described, for example, in U.S. Pat. No. 6,419,709 (Mao et al., “Biodegradable Terephthalate Polyester-Poly(Phosphite) Compositions, Articles, and Methods of Using the Same). According to this embodiment, the polymeric material comprises recurring monomeric units of the following formula V: 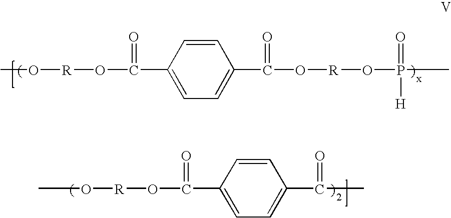
wherein R is a divalent organic moiety. R can be any divalent organic moiety so long as it does not interfere with the polymerization, copolymerization, or biodegradation reactions of the copolymer. Specifically, R can be an aliphatic group, for example, alkylene, such as ethylene, 1,2-dimethylethylene, n-propylene, isopropylene, 2-methylpropylene, 2,2-dimethylpropylene or tert-butylene, tert-pentylene, n-hexylene, n-heptylene, and the like; alkenylene, such as ethenylene, propenylene, dodecenylene, and the like; alkynylene, such as propynylene, hexynylene, octadecynylene, and the like; an aliphatic group substituted with a non-interfering substituent, for example, hydroxy-, halogen-, or nitrogen-substituted aliphatic group; or a cycloaliphatic group such as cyclopentylene, 2-methylcyclopentylene, cyclohexylene, and the like.

R can also be a divalent aromatic group, such as phenylene, benzylene, naphthalene, phenanthrenylene, and the like, or a divalent aromatic group substituted with a non-interfering substituent. Further, R can also be a divalent heterocyclic group, such as pyrrolylene, furanylene, thiophenylene, alkylene-pyrrolylene-alkylene, pyridylene, pyridinylene, pyrimidinylene, and the like; or can be any of these substituted with a non-interfering substituent.

Preferably, however, R is an alkylene group, a cycloaliphatic group, a phenylene group, or a divalent group having the formula VI: 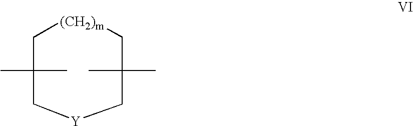
wherein Y is oxygen, substituted nitrogen, or sulfur, and m is 1 to 3. In some preferred embodiments, R is an alkylene group having 1 to 7 carbon atoms and, preferably, R is an ethylene group.

The value of x can vary depending upon the desired solubility of the polymer, the desired Tg, the desired stability of the polymer, the desired stiffness of the final polymers, and the biodegradability and release characteristics desired in the polymer. In general, x is 1 or more, and typically, x varies between 1 and 40. In some preferred embodiments, x is in the range of 1 to 30, preferably in the range of 1 to 20, or in the range of 2 to 20.

The number n can vary greatly depending upon the biodegradability and the release characteristics desired in the polymer, but typically varies from about 3 to about 7,500, preferably between 5 and 5,000. In some preferred embodiments, n is in the range of about 5 to about 300, or in the range of about 5 to about 200.

The most common way of controlling the value of x is to vary the feed ratio of the “x” portion relative to the monomer. For example, in the case of making the polymer VII: 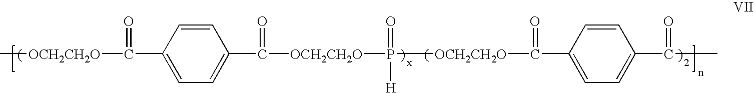
widely varying feed ratios of the dialkyl phosphite “x” reactant can be used with the diol reactant. Feed ratios of the reactants can easily vary from 99:1 to 1:99, for example, 95:5, 90:10, 85:15, 80:20, 75:25, 70:30, 65:35, 60:40, 55:45, 50:50, 45:55, 20:80, 15:85, and the like. Preferably, the feed ratio between the dialkyl phosphite reactant and the diol reactant varies from about 90:10 to about 50:50, or from about 85:15 to about 50:50, or from about 80:20 to about 50:50.

The most common general reaction in preparing a poly(phosphite) is a condensation of a diol with a dialkyl or diaryl phosphite according to the following equation:

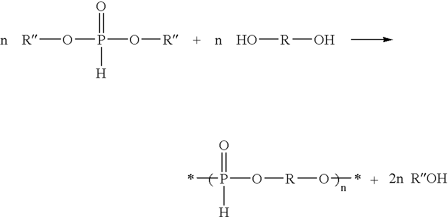

Poly(phosphites) can also be obtained by employing tetraalkyldiamides of phosphorus acid as condensing agents, according to the following equation:

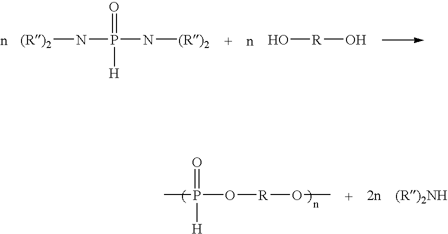

The above polymerization reactions can be either in bulk or solution polymerization. An advantage of bulk polycondensation is that it avoids the use of solvents and large amounts of other additives, thus making purification more straightforward. It can also provide polymers of reasonably high molecular weight.

Typical solvents for solution polycondensation include chlorinated organic solvents, such as chloroform, dichloromethane, or dichloroethane. The solution polymerization is preferably run in the presence of equimolar amounts of the reactants and a stoichiometric amount of an acid acceptor, usually a tertiary amine such as pyridine or triethylamine. The product is then typically isolated from the solution by precipitation with a nonsolvent and purified to remove the hydrochloride salt by conventional techniques known to those of ordinary skill in the art, such as by washing with an aqueous acidic solution, such as dilute hydrochloric acid.

Interfacial polycondensation can be used when high molecular weight polymers are desired at high reaction rates. Mild conditions minimize side reactions. Also, the dependence of high molecular weight on stoichiometric equivalence between diol and phosphite inherent in solution methods is removed. However, hydrolysis of the acid chloride may occur in the alkaline aqueous phase. Phase transfer catalysts, such as crown ethers or tertiary ammonium chloride, can be used to bring the ionized diol to the interface to facilitate the polycondensation reaction. The yield and molecular weight of the resulting polymer after interfacial polycondensation can be affected by reaction time, molar ratio of the monomers, volume ratio of the immiscible solvents, the type of acid acceptor, and the type and concentration of the phase transfer catalyst.

In a preferred embodiment, the process of making the biodegradable terephthalate polymer of formula V comprises the steps of polymerizing p moles of a diol compound having formula VIII: 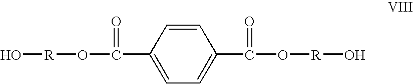
wherein R is as defined above for formula VI, with q moles of dialkyl or diaryl of formula IX: 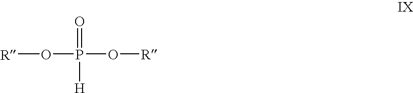
wherein p>q, to form q moles of a homopolymer of formula X, shown below: 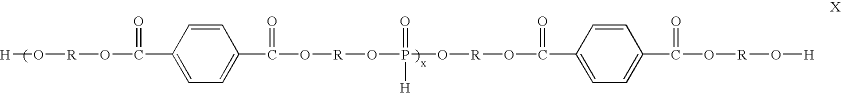
wherein R and x are as defined above for polymers V and VIII. The homopolymer so formed can be isolated, purified and used as is. Alternatively, the homopolymer, isolated or not, can be used to prepare a block copolymer composition of the invention by the steps of: (a) polymerizing as described above, and (b) further reaction the homopolymer of formula X with (p−q) moles of terephthaloyl chloride having the formula XI: 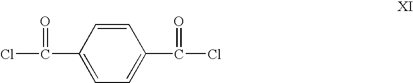
to form the copolymer of formula V.

The polymerization step (a) can take place at widely varying temperatures, depending upon the solvent used, the solubility desired, the molecular weight desired, and the susceptibility of the reactants to form side reactions. Preferably, however, the polymerization step (a) takes place at a temperature in the range of about −40° C. to about 160° C.; for solution polymerization, at a temperature in the range of about 0° C. to about 65° C.; for bulk polymerization, at temperatures of approximately 150° C.

The time required for the polymerization step (a) also can vary widely, depending upon the type of polymerization being used and the molecular weight desired. Preferably, however, the polymerization step (a) takes place in about 30 minutes to about 24 hours.

While the polymerization step (a) can be in bulk, in solution, by interfacial polycondensation, or any other convenient method of polymerization, preferably, the polymerization step (a) is a solution polymerization reaction. Particularly when solution polymerization reaction is used, an acid acceptor is advantageously present during the polymerization step (a). A particularly suitable class of acid acceptor comprises tertiary amines, such as pyridine, trimethylamine, triethylamine, substituted anilines, and substituted aminopyridines. The most preferred acid acceptor is the substituted aminopyridine 4-dimethyl-aminopyridine (“DMAP”).

The purpose of the copolymerization of step (b) is to form a block copolymer comprising (i) the phosphorylated homopolymer chains produced as a result of polymerization step (a), and (ii) interconnecting polyester units. The result is a block copolymer having a microcrystalline structure particularly well-suited to use as a controlled release biodegradable composition.

The copolymerization step (b) of the invention usually takes place at a slightly higher temperature than the temperature of the polymerization step (a), but also can vary widely, depending upon the type of copolymerization reaction used, the presence of one or more catalysts, the molecular weight desired, the solubility desired, and the susceptibility of the reactants to undesirable side reaction. However, when the copolymerization step (b) is carried out as a solution polymerization reaction, it typically takes place at a temperature in the range of about −40° C. to about 100° C. Typical solvents include methylene chloride, chloroform, or any of a wide variety of inert organic solvents.

The time required for the copolymerization of step (b) can also vary widely, depending upon the molecular weight of the material desired and, in general, the need to use more or less rigorous conditions for the reaction to proceed to the desired degree of completion. Typically, however, the copolymerization step (b) takes place during a time of about 30 minutes to about 24 hours.

The terephthalate-poly(phosphite) polymer produced, whether a homopolymer or a block copolymer, is isolated from the reaction mixture by conventional techniques, such as by precipitating out, extraction with an immiscible solvent, evaporation, filtration, crystallization, and the like. Typically, however, the polymer of formula V is both isolated and purified by quenching a solution of said polymer with a non-solvent or a partial solvent, such as diethyl ether or petroleum ether.

In another embodiment, the terephthalate polyester includes a phosphoester linkage that is a phosphonate. Suitable terephthalate polyester-poly(phosphonate) copolymers are described, for example, in U.S. Pat. Nos. 6,485,737 and 6,153,212 (Mao et al., “Biodegradable Terephthalate Polyester-Poly(Phosphonate) Compositions, Articles and Methods of Using the Same). According to this embodiment, the polymeric material comprises recurring monomeric units shown in Formula XII: 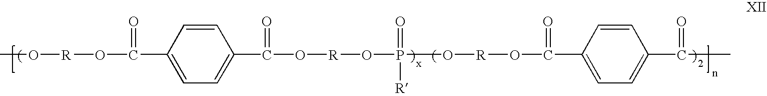
wherein R is a divalent organic moiety as defined above for terephthalate poly(phosphites) of formula V. R′ in the polymeric material of this embodiment is an aliphatic, aromatic, or heterocyclic residue. When R′ is aliphatic, it is preferably alkyl, such as methyl, ethyl, n-propyl, isopropyl, n-butyl, tert-butyl, —C₈H₇, and the like; or alkyl substituted with a non-interfering substituent, such as halogen, alkoxy, or nitro.

When R′ is aromatic, it typically contains about 5 to about 14 carbon atoms, or about 5 to about 12 carbon atoms and, optionally, can contain one or more rings that are fused to each other. Examples of particularly suitable aromatic groups include phenyl, naphthyl, anthracenyl, phenanthranyl, and the like.

When R′ is heterocyclic, it typically contains about 5 to 14 ring atoms, preferably about 5 to 12 ring atoms, and one or more heteroatoms. In one preferred embodiment, R′ is an alkyl group or a phenyl group and, even more preferably, an alkyl group having 1 to 7 carbon atoms. Preferably, R′ is an ethyl group.

The value of x can be varied as described above for polymeric material containing phosphite ester linkages. Similarly, one method for controlling the value of x is to vary the feed ratio of the “x” portion relative to the monomer. In this particular embodiment, feed ratios of the ethyl phosphonic dichloride “x” reactant (“EP”) can be used with the terephthaloyl chloride reactant (“TC”) to manufacture the polymer of formula XIII:

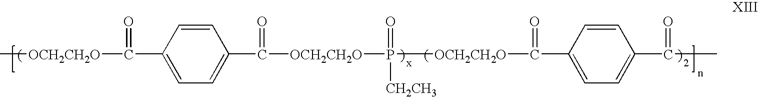

The most common general reaction in preparing a poly(phosphonate) is a dehydrochlorination between a phosphonic dichloride and a diol according to the following equation:

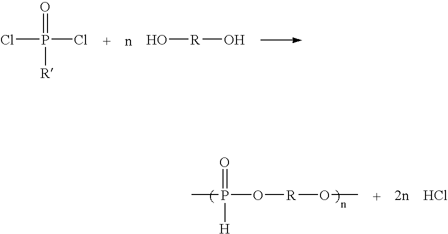

Bulk polycondensation, solution polycondensation, or interfacial polycondensation can be used to synthesize the polymers. A Friedel-Crafts reaction can also be used to synthesize poly(phosphonates). Polymerization typically is effected by reacting either bis(chloromethyl) compounds with aromatic hydrocarbons or chloromethylated diphenyl ether with triaryl phosphonates. Poly(phosphonates) can also be obtained by bulk condensation between phosphorus diimidazolides and aromatic diols, such as resorcinol and quinoline, usually under nitrogen or some other inert gas.

In one preferred embodiment, the process of making the biodegradable terephthalate polymer of formula XIII comprises the steps of polymerizing p moles of a diol compound having formula VIII above, with q moles of a phosphonic dichloride of formula XIV: 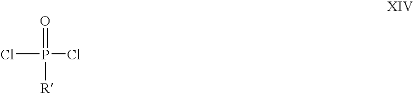
Wherein R′ is defined as above, and p>q, to form q moles of a homopolymer of formula XV shown below: 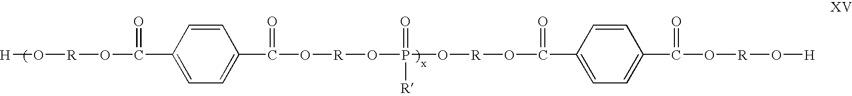
wherein R, R′ and x are as defined above. The homopolymer so formed can be isolated, purified and used as is. Alternatively, the homopolymer, isolated or not, can be used to prepare a block copolymer composition of the invention by: (a) polymerizing as described above, and (b) further reacting the homopolymer of formula XV and excess diol of formula VIII with (p−q) moles of terephthaloyl chloride having the formula XVI: 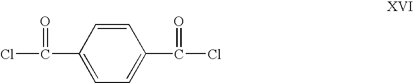
to form the copolymer of formula XII.

The function of the polymerization reaction of step (a) is to phosphorylate the di-ester starting material and then to polymerize it to form the homopolymer. As described above for polymeric material containing phosphite ester linkages, the polymerization step (a) can take place at widely varying temperatures and times.

The addition sequence of the polymerization step (a) can vary significantly depending upon the relative reactivities of the diol of formula VIII, the phosphonic dichloride of formula XIV, and the homopolymer of formula XV; the purity of these reactants; the temperature at which the polymerization reaction is performed; the degree of agitation used in the polymerization reaction; and the like. Preferably, however, the diol of formula VIII is combined with a solvent and an acid acceptor, and the phosphonic dichloride is added slowly, for example, a solution of the phosphonic dichloride in a solvent can be trickled in or added dropwise to the chilled reaction mixture of diol, solvent, and acid acceptor, the control the rate of the polymerization reaction.

The purpose and conditions of the copolymerization of step (b) are as described above for polymeric material containing phosphite ester linkages.

The polymer of formula XII, whether a homopolymer or a block polymer, is isolated from the reaction mixture by conventional techniques, such as by precipitating out, extraction with an immiscible solvent, evaporation, filtration, crystallization, and the like. Typically, however, the polymer of formula XII is both isolated and purified by quenching a solution of the polymer with a non-solvent or a partial solvent, such as diethyl ether or petroleum ether.

The polymer of formula XII is usually characterized by a release rate of the bioactive agent in vivo that is controlled at least in part as a function of hydrolysis of the phosphoester bond or the polymer during biodegradation.

Further, the structure of the side chain can influence the release behavior of the polymer. For example, it is expected that conversion of the phosphorus side chain to a more lipophilic, more hydrophobic or bulky group would slow down the degradation process. Thus, for example, release is usually faster from copolymer compositions with a small aliphatic group side chain than with a bulky aromatic side chain.

In another embodiment, the terephthalate polyester includes a phosphoester linkage that is a phosphate. Suitable terephthalate polyester-poly(phosphate) copolymers are described, for example, in U.S. Pat. Nos. 6,322,797 and 6,600,010 (Mao et al., “Biodegradable Terephthalate Polyester-Poly(Phosphate) Polymers, Compositions, Articles, and Methods for Making and Using the Same). According to this embodiment, the polymeric material comprises recurring monomeric units shown in Formula XVII: 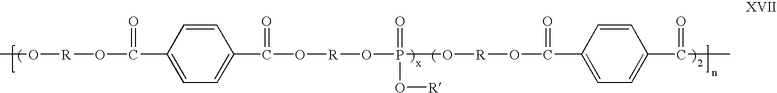
wherein R is a divalent organic moiety as described above for terephthalate poly(phosphites) of Formula V and terephthalate poly(phosphonates) of Formula XII. Preferably, R is an alkylene group, a cycloaliphatic group, a phenylene group, or a divalent group of the formula XVIII: 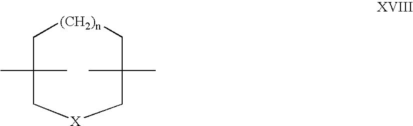
wherein X is oxygen, substituted nitrogen, or sulfur, and n is 1 to 3. Preferably, R is an alkylene group having 1 to 7 carbon atoms and, preferably, R is an ethylene group, a 2-methyl-propylene group, or a 2,2′-dimethylpropylene group. R′ is as describe above for terephthalate poly(phosphites) of Formula V and terephthalate poly(phosphonates) of Formula XII, with the proviso that R′ could also comprise an alkyl conjugated to a biologically active substance to form a pendant bioactive agent delivery system. The value x is 1 or more and can vary as described for terephthalate poly(phosphites) of Formula V and terephthalate poly(phosphonates) of Formula XII. Similarly, one method for controlling the value of x is to vary the feed ratio of the “x” portion relative to the other monomer (for example, varying the feed ratios of the ethyl phosphorodichloridate “x” reactant (“EOP”) relative to the terephthaloyl chloride reactant (“TC”)). The value n is 1 to 5,000.

The most common general reaction in preparing poly(phosphates) is a dehydrochlorination between a phosphodichlorinate and a diol according to the following equation:

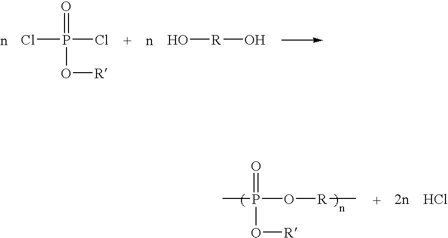

A Friedel-Crafts reaction can also be used to synthesize poly(phosphates). The principals described above for poly(phosphonates) can be utilized for synthesis of poly(phosphates) as well. The poly(phosphates) can be synthesized via bulk polycondensation, solution polycondensation, and interfacial polycondensation as described herein.

In a preferred embodiment, the process of making a biodegradable terephthalate homopolymer of formula XVII comprises the step of polymerizing p moles of a diol compound having formula XIX: 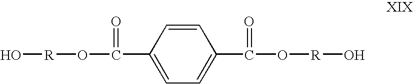
wherein R is as defined above, with q moles of a phosphorodichloridate of formula XX: 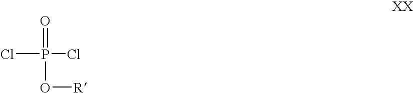
wherein R′ is defined above, and p>q, to form q moles of a homopolymer of formula XXI as shown below: 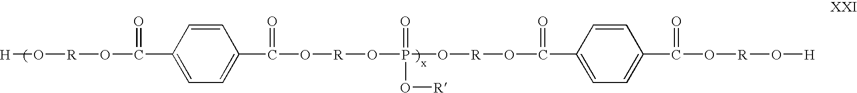
wherein R, R′ and x are as defined above. The homopolymer so formed can be isolated, purified and used as is. Alternatively, the homopolymer, isolated or not, can be used to prepare a block copolymer by (a) polymerizing as described above, and (b) further reacting the homopolymer of formula XXI and excess diol of formula XIX with (p−q) moles of terephthaloyl chloride having the formula XVI to form the polymer of formula XVII.

The function of polymerization steps (a) and (b), as well as conditions therefor are as described above for poly(phosphonates). The addition sequence for the copolymerization step (b) can vary significantly depending upon the relative reactivities of the homopolymer of formula XXI and the terephthaloyl chloride of formula XVI; the purity of these reactants; the temperature at which the copolymerization reaction is performed; the degree of agitation used in the copolymerization reaction; and the like. Preferably, however, the terephthaloyl chloride of formula XVI is added slowly to the reaction mixture, rather than vice versa. For example, a solution of the terephthaloyl chloride in a solvent can be trickled in or added dropwise to the chilled or room temperature reaction, to control the rate of the copolymerization reaction.

The polymeric materials comprising a biodegradable terephthalate copolymer that includes a phosphorus-containing linkage (poly(phosphates), poly(phosphonates) and poly(phosphites)) can comprise additional biocompatible monomeric units so long as they do not interfere with the biodegradable characteristics of the polymeric material. Such additional monomeric units can, in some embodiments, offer even greater flexibility in designing the precise release profile desired for targeted bioactive agent delivery or the precise rate of biodegradability. Examples of such additional biocompatible monomers include, but are not limited to, the recurring units found in polycarbonates, polyorthoesters, polyamides, poly(iminocarbonates), and polyanhydrides.

The polymeric material of these embodiments is preferably soluble in one or more common organic solvents for ease of fabrication and processing. Common organic solvents can include chloroform, dichloromethane, acetone, ethyl acetate, DMAC, N-methylpyrrolidone, dimethylformamide, and dimethylsulfoxide. The polymeric material is preferably soluble in at least one of these solvents.

The Tg of the polymeric material according to these embodiments can vary widely depending upon the branching of the diols used to prepare the polymer, the relative proportion of phosphorus-containing monomer used to make the polymer, and the like. However, preferably, the Tg is within the range of about −10° C. to about 100° C., or in the range of about 0° C. to about 50° C.

When working with poly(phosphates) and poly(phosphonates), the structure of the side chain can influence the release behavior of the polymer. For example, it is generally expected that, with the classes of poly(phosphoesters) described herein, conversion of the phosphorus side chain to a more lipophilic, more hydrophobic or bulky group would slow down the degradation process. For example, release would usually be faster from copolymer compositions with a small aliphatic group side chain than with a bulky aromatic side chain.

The terephthalate poly(phosphites) of formula V are usually characterized by a release rate of the bioactive agent in vivo that is controlled at least in part as a function of hydrolysis of the phosphoester bond of the polymer during biodegradation. However, poly(phosphites) do not have a side chain that can be manipulated to influence the rate of biodegradation.

In still further embodiments of the invention, the first polymer comprises a copolymer comprising a biodegradable, segmented molecular architecture that includes at least two different ester linkages. According to these particular embodiments, the first polymer can comprise block copolymers (of the AB or ABA type) or segmented (also known as multiblock or random-block) copolymers of the (AB)ₙ type. These copolymers are formed in a two (or more) stage ring opening copolymerization using two (or more) cyclic ester monomers that form linkages in the copolymer with greatly different susceptibilities to transesterification. These embodiments are described, for example, in U.S. Pat. No. 5,252,701 (Jarrett et al., “Segmented Absorbable Copolymer”) and will now be described in some detail herein.

In one aspect, the first polymer comprises a copolymer comprising a biodegradable, segmented molecular architecture that includes at least two different ester linkages. Generally speaking, the segmented molecular architecture comprises a plurality of fast transesterifying linkages and a plurality of slow transesterifying linkages. The fast transesterifying linkages have a segment length distribution of greater than 1.3. Sequential addition copolymerization of cyclic ester monomers is utilized in conjunction with a selective transesterification phenomenon to create biodegradable copolymer molecules with specific architectures.

According to the invention, the copolymer can be manufactured by sequential addition of at least two different cyclic ester monomers in at least two stages. The first cyclic ester monomer is selected from carbonates and lactones, and mixtures thereof. The second cyclic ester monomer is selected from lactides and mixtures thereof. The sequential addition comprises the following steps:
(1) first polymerizing a first stage at least the first cyclic ester monomer in the presence of a catalyst at a temperature in the range of about 160° C. to about 220° C. to obtain a first polymer melt;
(2) adding at least the second cyclic ester monomer to the first polymer melt; and
(3) copolymerizing in a second stage the first polymer melt with at least the second cyclic ester monomer to obtain a second copolymer melt.

The process also comprises transesterifying the second copolymer melt for up to about 5 hours at a temperature of greater than about 180° C.

Another process for manufacturing a copolymer having a biodegradable, segmented molecular architecture comprises sequential addition of at least two different cyclic ester monomers in three stages. The first cyclic ester monomer is selected from carbonates, lactones, and mixtures of carbonates and lactones. The second cyclic ester monomer is selected from lactides and mixtures thereof. The sequential addition comprises the following steps:
(1) first polymerizing in a first stage at least the first cyclic ester monomer in the presence of a catalyst at a temperature in the range of about 160° C. to about 220° C. to obtain a first polymer melt;
(2) first adding at least the second cyclic ester monomer to the first polymer melt;
(3) second copolymerizing in a second stage the first polymer melt with at least the second cyclic ester monomer to obtain a second copolymer melt;
(4) second adding at least the second cyclic ester monomer to the second copolymer melt; and
(5) copolymerizing in a third stage the second copolymer melt with at least the second cyclic ester monomer to obtain a third copolymer melt.

The process also comprises transesterifying the third copolymer melt from up to about 5 hours at a temperature of greater than about 180° C.

Optionally, the process can involve polymerization in the presence of a metal coordination catalyst and/or an initiator. In some embodiments, the initiator can be selected from monofunctional and polyfunctional alcohols. It is generally preferred to conduct the sequential polymerization in a single reaction vessel, by sequentially adding the monomers thereto. However, if desired, one or more of the stages can be polymerized in separate reaction vessels, finally combining the stages for transesterification in a single reaction vessel. Such a process would allow the use of a cyclic polyester forming monomers for one or more of the stages.

Transesterification in aliphatic polyesters derived from cyclic monomers is known in the art. For example, Gnanou and Rempp, Macromol. Chem., 188:2267-2275 (1987) have described the anion polymerization of ε-caprolactone in the presence of lithium alkoxides as being a living polymerization that is accompanied by simultaneous reshuffling. According to this reference, if reshuffling occurs between two different molecules, it can be referred to as “scrambling.” If reshuffling occurs intramolecularly, it is called back-biting, and it results in the formation of cycles, the remaining linear macromolecules are of lower molecular weight, but they still carry an active site at the chain end.

In still further embodiments, the first polymer comprises a random copolymer comprising at least one carbonate unit as the major component, the carbonate copolymerized with at least one second monomeric component. According to these embodiments, certain aliphatic carbonates can form highly crystalline random copolymers with other monomer components, so long as the appropriate carbonate is present as the major component. These copolymers can provide one or more advantages, such as relatively high modulus and tensile strength, controllable biodegradation rates, blood compatibility, and biocompatibility with living tissue. In preferred aspects, these copolymers also induce minimal inflammatory tissue reaction, as biodegradation of the carbonate polymer by hydrolytic depolymerization results in degradation substances having physiologically neutral pH. Exemplary random copolymers are described, for example, in U.S. Pat. No. 4,891,263 (Kotliar et al.), U.S. Pat. No. 5,120,802 (Mares et al.), U.S. Pat. No. 4,916,193 (Tang et al.), U.S. Pat. No. 5,066,772 (Tang et al.), and U.S. Pat. No. 5,185,408 (Tang et al.).

According to these embodiments, the copolymers are random copolymers comprising as a minor component one or more recurring monomeric units, and as a major component, a recurring carbonate monomeric unit of the following general structures (XXII): 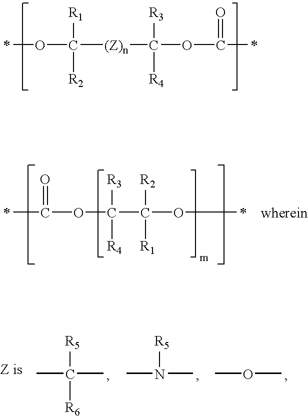
or combinations thereof, where Z is selected such that there are no adjacent heteroatoms;

n and m are the same or different and are integers from about 1 to about 8; and

R₁, R₂, R₃, and R₄ are the same or different at each occurrence and are hydrogen, alkoxyaryl, aryloxyaryl, arylalkyl, alkylarylalkyl, arylalkylaryl, alkylaryl, arylcarbonylalkyl, aryloxyalkyl, alkyl, aryl, alkylcarbonylalkyl, cycloalkyl, arylcarbonylaryl, alkylcarbonylaryl, alkoxyalkyl, or aryl or alkyl substituted with one or more biologically compatible substituents such as alkyl, aryl, alkoxy, aryloxy, dialkyamino, diarylamino, alkylarylamino substituents; R₅ and R₆ are the same or different and are R₁, R₂, R₃, R₄, dialkylamino, diarylamino, alkylarylamino, alkoxy, aryloxy, alkanoyl, or arylcarbonyl; or any two of R₁ to R₆ together can form an alkylene chain completing a 3, 4, 5, 6, 7, 8, or 9 membered monocyclic, alicyclic, spiro, bicyclic, and/or tricyclic ring system, which system can optionally include one or more non-adjacent carbonyl, oxa, alkylaza, or arylaza groups; with the proviso that at least one of R₁ to R₆ is other than hydrogen.

Illustrative of useful R₁, R₂, R₃, and R₄ groups are hydrogen; alkyl such as methyl, ethyl, propyl, butyl, pentyl, octyl, nonyl, tert-butyl, neopentyl, isopropyl, sec-butyl, dodecyl, and the like; cycloalkyl such as cyclohexyl, cyclopentyl, cyclooctyl, cycloheptyl, and the like; alkoxyalkylene such as methoxymethylene, ethoxymethylene, butoxymethylene, propoxyethylene, pentoxybutylene, and the like; aryloxyalkylene and aryloxyarylene such as phenoxyphenylene, phenoxymethylene and the like; and various substituted alkyl and aryl groups such as 4-dimethylaminobutyl, and the like.

Illustrative of other R₁ to R₄ groups are divalent aliphatic chains, which can optionally include one or more oxygen, trisubstituted amino or carbonyl groups, such as —(CH₂)₂—, —CH₂(O)CH₂—, —(CH₂)₃—, —CH₂—H(CH₃)—, —(CH₂)₄—, —(CH₂)₅—, —CH₂OCH₂—, —(CH₂)₂—N(CH₃)CH₂—, —CH₂C(O)CH₂—, —(CH₂)₂—N(CH₃)—(CH₂)₂—, and the like, and divalent chains to form fused, spiro, bicyclic or tricyclic ring systems, such as —CH(CH₂CH₂)₂CH—, —CH(CH₂CH₂CH₂)₂CH—, —CH(CH₂)(CH₂CH₂)CH—, —CH(CH₂)(CH₂—CH₂CH₂)CH—, —CH(C(CH₃)₂)(CH₂CH₂)CH—, and the like.

Illustrative of useful R₅ and R₆ groups are the above-listed representative R₁ to R₄ groups, including —OCH₂C(O)CH₂—, —(CH₂)₂—NCH₃—, —OCH₂C(O)CH₂—, (CH₂)₂, alkoxy such as propoxy, butoxy, methoxy, isopropoxy, pentoxy, nonyloxy, ethoxy, octyloxy, and the like; dialkylamino such as dimethylamino, methylethylamino, diethylamino, dibutylamino, and the like; alkanoyl such as propanoyl, acetyl, hexanoyl, and the like; arylcarbonyl such as phenylcarbonyl, p-methylphenyl carbonyl, and the like; and diarylamino and arylalkylamino such as diphenylamino, methylphenylamino, ethylphenylamino, and the like.

Preferred for use in accordance with these embodiment are random copolymers comprising as a major component, carbonate recurring units of structure XXIIA, wherein Z is —(R₅—C—R₆)—, or a combination thereof; n is 1, 2, or 3; and R₁ to R₆ are as defined above, preferably where aliphatic moieties included in R₁ to R₆ include up to about 10 carbon atoms and the aryl moieties include up to about 16 carbon atoms.

Illustrative of these preferred copolymers are those wherein, in the major component, n is 1 and Z is of the formula XXIII: 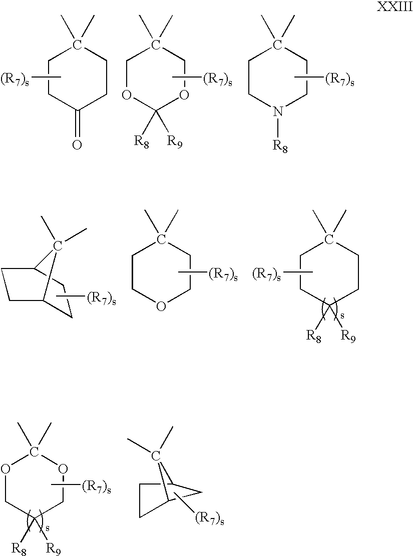
where —C— denotes the center carbon atom of Z, when Z is —C(R₅)(R₆)—; R₇ is the same or different and is aryl, alkyl or an alkylene chain completing a 3 to 16 membered ring structure, including fused, spiro, bicyclic and/or tricyclic structures, and the like; R₈ and R₉ are the same or different at each occurrence and are R₇ or hydrogen, and s is the same or different at each occurrence and is 0 to 3, and the open valencies are substituted with hydrogen atoms.

Also illustrative of these preferred major components are those comprising recurring units of the formula XXIV: 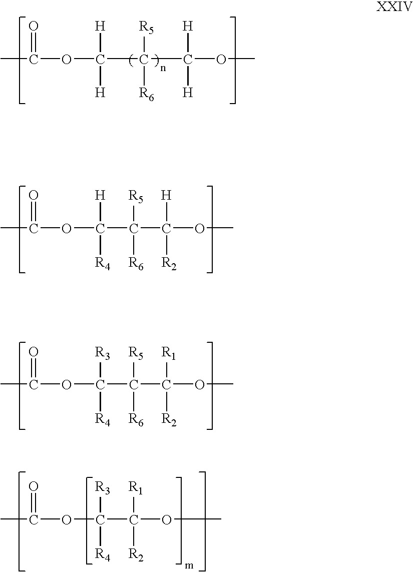
wherein:

R₁, R₂, R₃, and R₄, are the same or different at each occurrence and are hydrogen, alkyl such as methyl, ethyl, n-propyl, isopropyl, n-butyl, sec-butyl, t-butyl, neopentyl, and the like; phenyl; anisyl; phenylalkyl, such as benzyl, phenethyl, and the like; phenyl substituted with one or more alkyl or alkoxy groups such as tolyl, xylyl, p-methoxyphenyl, m-ethoxyphenyl, p-propoxyphenyl, and the like; and alkoxyalkyl such as methoxymethyl, ethoxymethyl, and the like; R₈ and R₆ are the same or different and are R₁ to R₄; alkoxy, alkanoyl, arylcarbonyl, dialklyamino; or any two of R₁ to R₆ together can form alkylene chain completing 4, 5, 6, 7, 8, or 9 membered monocyclic, spiro, bicyclic and/or tricyclic ring structure which structure can optionally include one or more non-adjacent divalent carbonyl, oxa, alkylaza, or arylaza groups with the proviso that at least one of R₁ or R₆ is other than hydrogen; and

n and m are the same or different and are 1, 2, or 3.

Particularly preferred for use in these embodiments are random copolymers comprising as a major component, recurring units of the formula XXV: 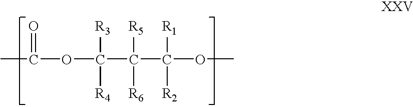
wherein:

R₁ to R₄ are the same or different and are alkyl, hydrogen, alkoxyalkyl, phenylalkyl, alkoxyphenyl, or alkylphenyl, wherein the aliphatic moieites include 1 to 9 carbon atoms; and

R₅ and R₆ are the same or different at each occurrence and are selected from the group of R₁ to R₄ substituents, aryloxy, and alkoxy, or R₅ and R₆ together can form an aliphatic chain completing a 3 to 1 membered spiro, bicyclic, and/or tricyclic structure which can include one or two non-adjacent oxa, alkylaza, or arylaza groups, with the proviso that at least one of R₁ to R₄ is other than hydrogen.

Preferably, the random copolymer comprises as a major component, recurring monomeric units of the following formula XXVI: 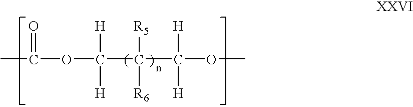
wherein:

n is 1;

R₅ and R₆ are the same or different and are hydrogen, phenyl, phenylalkyl, or phenyl or phenylalkyl substituted with one or more alkyl or alkoxy groups; or alkyl or R₅ and R₆ together make a divalent chain forming a 3 to 6 membered spiro, bicyclic, and/or tricyclic ring structure which can include one or two non-adjacent carbonyl, oxa, alkylaza, or arylaza groups, with the proviso that at least one of R₅ and R₆ is other than hydrogen.

It is preferred that the random copolymer comprises as a major component, recurring monomeric units of Formula XXVI, particularly when R₅ and R₆ are the same or different and are alkyl, phenyl, phenylalkyl, or phenyl or phenylalkyl substituted with one or more alkyl or alkoxy groups; or a divalent chain forming a 3 to 10 membered, preferably 5 to 7 membered, spiro or bicyclic ring structure that can optionally include one or two non-adjacent oxa, carbonyl, or disubstituted amino groups. It can be particularly preferred that R₅ and R₆ are the same or different and are phenyl, alkylphenyl or phenylalkyl such as tolyl beneyl, phenethyl or phenyl, or lower alkyl of 1 to 7 carbon atoms such as methyl, ethyl, propyl, isopropyl, n-butyl, tertiary butyl, pentyl, neopentyl, hexyl, and secondary butyl.

In most preferred embodiments utilizing Formula XXVI, R₅ and R₆ are the same or different, and are lower alkyl having about 1 to about 4 carbon atoms, and do not differ from each other by more than about 3 carbon atoms, and preferably by not more than about 2 carbon atoms. It is preferred that R₅ and R₆ be the same and comprise alkyl of about 1 to 2 carbon atoms, and preferably methyl for each of R₅ and R₆.

According to these embodiments, the copolymers include a minor component comprising one or more other recurring monomer units. The minor component of the random copolymers of the invention can vary widely. It is preferred that the minor component is also biodegradable and bioresorbable.

Illustrative of the second recurring monomeric components are those derived from carbonates, including but not limited to certain of the monomeric units included within the scope of Formula XXIIA wherein n is 1 to 8 within (Z)ₙ, and Formula XXIIB and Formula XXVI, wherein n=1, particularly those less preferred as the major component, and those derived from substituted or nonsubstituted ethylene carbonates, tetramethylene carbonates, trimethylene carbonates, pentamethylene carbonates, and the like. Also illustrative of the second recurring monomeric unit are those that are derived from monomers that polymerize by ring opening polymerization as, for example, substituted and unsubstituted beta, gamma, delta, omega, and other lactones such as those of the formula XXVII: 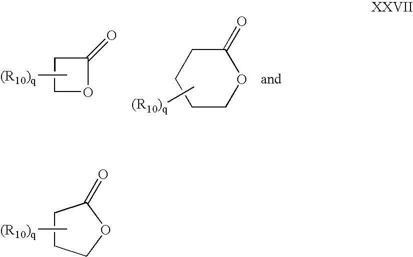
where R₁₀ is alkoxy, alkyl or aryl, and q is 0 to 3, wherein the open valencies are substituted with hydrogen atoms. Such lactones include caprolactones, valerolactones, butyrolactones, propiolactones, and the lactones of hydroxy carboxylic acids such as 3-hydroxy-2-phenylpropanoic acid, 3-hydroxy-3-phenylpropanoic acid, 3-hydroxybutanoic acid, 3-hydroxy-3-methylbutanoic acid, 3-hydroxypentanoic acid, 5-hydroxypentanoic acid, 3-hydroxy-4-methylheptanoic acid, 4-hydroxyocatnoic acid, and the like; and lactides such as L-lactide, D-lactide, D,L-lactide; glycolide; and dilactones such as those of the formula XXVIII: 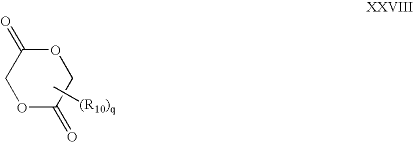
where R₁₀ and q are as defined above in formula XXVII and where the open valencies are substituted with hydrogen atoms. Such dilactones include the dilactones of 2-hydroxybutyric acid, 2-hydroxy-2-phenylpropanoic acid, 2-hydroxyl-3-methylbutanoic acid, 2-hydroxypentanoci acid, 2-hydroxy-4-methylpentanoic acid, 2-hydroxyhexanoic acid, 2-hydroxyoctanoic acid, and the like.

Illustrative of still further useful minor components are units derived from dioxepanones such as those described in U.S. Pat. No. 4,052,988 and U.K. Patent No. 1,273,733. Such dioxepanones include alkyl and aryl substituted and unsubstituted dioxepanones of the formula XXIX:

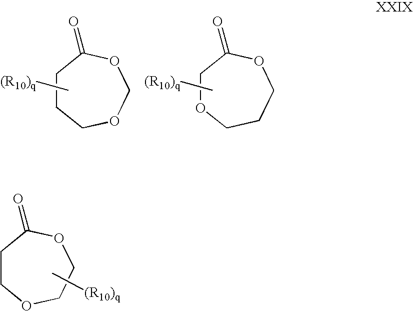

and monomeric units derived from dioxanones such as those described in U.S. Pat. Nos. 3,952,016, 4,052,988, 4,070,375, and 3,959,185, as for example, alkyl or aryl substituted and unsubstituted dioxanones of the formula XXX: 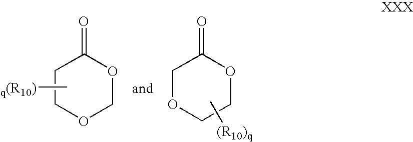
wherein q is as defined above; R₁₀ is the same or different at each occurrence and are hydroxycarbonyl groups such as alkyl and substituted alkyl, and aryl or substituted aryl; and the open valencies are substituted with hydrogen atoms. Preferably R₁₀ is the same or different and are alkyl groups containing 1 to 6 carbon atoms, preferably 1 or 2 carbon atoms, and q is 0 or 1.

Suitable minor components also include monomeric units derived from ethers such as 2,4-dimethyl-1,3-dioxane, 1,3-dioxane, 1-,4-dioxane, 2-methyl-5-methoxy-1,3-dioxane, 4-methyl-1,3-dioxane, 4-methyl-4-phenyl-1,3-dioxane, oxetane, tetrahydrofuran, tetrahydropyran, hexamethylene oxide, heptamethylene oxide, octamethylene oxide, nonamethylene oxide, and the like.

Still further minor components include monomeric units derived from epoxides such as ethylene oxide, propylene oxide, alkyl substituted ethylene oxides such as ethyl, propyl, and butyl substituted ethylene oxide, the oxides of various internal olefins such as the oxides of 2-butene, 2-pentene, 2-hexene, 3-hexene, an like epoxides; and also including units derived from epoxides with carbon dioxide; and monomeric units derived from orthoesters or orthocarbonates such as alkyl or aryl substituted or unsubstituted orthoesters, orthocarbonates, and cyclic anhydrides which may optionally include one or more oxa, alkylaza, arylaza, and carbonyl groups of the formula XXXI: 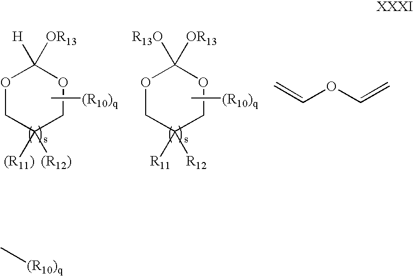
where q and R₁₀ are as described above, r is o to about 10, R₁₃ is the same or different at each occurrence and is alkyl or aryl, and R₁₁ and R₁₂ are the same or different and are hydrogen, alkyl or aryl.

Relative percentages of each of the recurring monomeric units that make up the copolymers of these embodiments can vary widely. The only requirement is that at least one type of recurring monomeric unit within the scope of Formula XXIIA be in the major amount, and that the other type of recurring unit or units be in the minor amount. As used herein, “major amount” is more than about 50 weight % based upon the total weight of all recurring monomeric units in the copolymer and “minor amount” is less than about 20 weight % based upon the total weight of all recurring monomeric units in the copolymer.

In addition, for certain applications, end-capping of these biopolymers can be desirable. End-capping can be accomplished by, for example, acylating, alkylating, silylating agents and the like.

Thus, the invention provides implantable devices (such as stents) that include a coating composition including a first coated layer comprising a first polymer and a second coated layer comprising a second polymer. The first biodegradable polymer is preferably a polyether ester copolymer, such as PEGT/PBT. Other suitable first polymers are described herein. The second polymer comprises a biodegradable polymer that is selected to provide controlled release of a bioactive agent. These aspects of the invention will now be described.

As illustrated in the Examples, when a single layer coating comprising PEGT/PBT alone is formulated with a small molecule bioactive agent (such as dexamethasone), the bioactive agent is quickly released from the coating. As illustrated in Example 1, for example, greater than 90% of dexamethasone is released within 24 hours from a coating composed of a single layer of PEGT/PBT and dexamethasone. However, in accordance with the invention, once a second (or even third, fourth, etc.) coated layer is provided in connection with the PEGT/PBT coated layer, the initial burst of bioactive agent can be controlled, allowing for more sustained release of bioactive agent for a longer period of time. Depending upon the second polymer chosen, and the relative location of the first and second polymers as compared to the device surface and/or bioactive agent, the release of small molecular weight bioactive agents can be significantly reduced within the first 24 hours. Following an initial release period, substantially linear release of bioactive agent can be achieved, thereby providing controlled release of the bioactive agent (other release profiles are also contemplated, in addition to linear release over time).

In accordance with the invention, a polyether ester copolymer (or other first polymer) is provided in a multiple layer arrangement with a second biodegradable polymer, to form a biodegradable coating that can controllably release bioactive agent when exposed to biological conditions. A wide variety of second polymers can be utilized in accordance with principles of the invention. Typically, the second polymer has a slower bioactive agent release rate relative to the first polymer. The second polymer can include organic esters or ethers, which when degraded result in physiologically acceptable degradation products. In addition, anhydrides, amides, orthoesters, or the like, can be used. The second polymer can be composed of addition or condensation polymers, crosslinked or non-crosslinked. For the most part, besides carbon and hydrogen, the polymers will include oxygen and nitrogen, particularly oxygen. The oxygen can be present as oxy (for example, hydroxy, ether, carbonyl, and the like), carboxylic acid ester, and the like. The nitrogen can be present as amide, cyano, or amino. Table 1 lists some known biodegradable polymers that can be used as the second polymer according to the invention. It is understood the invention is not limited to the polymers listed in the table; rather, this list is illustrative.

TABLE 1
Representative Second Biodegradable Polymers

Synthetic
Polypeptides
Polydepsipeptides
Polyamides
Aliphatic polyesters
Polyglycolide (PGA) and copolymers (including PEG copolymers)
Polylactide (PLA) and copolymers (including PEG copolymers)
Polyanhydrides
Poly(alkylene succinates)
Poly(hydroxy butyrate) (PHB)
Poly(caprolactone) and copolymers
Poly(butylene diglycolate)
Polydihydropyrans
Polyphosphazenes
Poly(ortho esters)
Polydioxanone (PDS)
Poly(phosphate esters)
Polyhydroxyvalerate
Poly(acetals)
Polypropylene fumarate
Trimethylene carbonates
Poly(ethyl glutamate-co-glutamic acid)
Poly(tert-butyloxy-carbonylmethyl glutamate)
Polybutyrates
Polycarbonates
Poly(ester-amides), including blends thereof

It is understood that poly(lactide) includes the naturally occurring isomer, poly(L-lactide) (PLLA), and poly(D,L-lactide) (PLA). Further, the polyanhydrides include poly[bis(p-carboxyphenoxy) propane]anhydride (PCPP) and poly(terephthalic anhydride (PTA)).

In some aspects, aliphatic polyesters can be useful second polymers. In some embodiments, aliphatic polyesters that are derived from monomers selected from lactic acid, glycolic acid, caprolactone, ethylene glycol, ethyloxyphosphate, and similar monomeric units, can be useful. These polymers can be homopolymers or copolymers. Illustrative aliphatic polyesters of this nature include, but are not limited to: poly(1,4-butylene adipate-co-polycaprolactam); polycaprolactone; polycaprolactone diol; polyglycolide; poly(DL-lactide); poly-L-lactide; poly(DL-lactide-co-caprolactone) (various mole % of DL-lactide); poly(L-lactide-co-caprolactone-co-glycolide) (various MW and various mole % of DL-lactide); and poly(DL-lactide-co-glycolide) (various MW and various mole % of DL-lactide).

In some aspects, polyphosphoesters can be useful as second polymers, since these polymers can exhibit many properties important for bioactive agent delivery. Polyphosphoesters biodegrade through hydrolysis and possibly enzymatic digestion, and many of these polymers and copolymers are soluble in a range of organic solvents, such as THF, chloroform, acetonitrile, and ethyl acetate. In some embodiments, polyphosphoesters including monomeric units of lactide and/or ethylene glycol can be useful. Useful polyphosphoesters in accordance with the principles of the invention possess a bioactive agent elution rate that is slower than the first polymer (polyether ester copolymer), to provide controlled release of bioactive agent as contemplated herein. In some aspects, useful polyphosphoesters are soluble in a common solvent for the first polymer and bioactive agent.

In further aspects, the second polymer itself can comprise a blend of polymers. For example, a blend of two or more poly(ester-amide) polymers (PEA) can be utilized, such as those described in U.S. Pat. No. 6,703,040 (Katsarava et al.). Such polymers can be prepared by polymerization of a diol (D), a dicarboxylic acid (C), and an alpha-amino acid (A) through ester and amide links in the form (DACA)ₙ. Illustrative amino acids include any natural or synthetic alpha-amino acid, in particular neutral amino acids.

According to these aspects, suitable diols include any aliphatic diol, such as alkylene diols like HO—(CH₂)ₖ—OH (i.e., non-branched), branched diols (such as propylene glycol), cyclic diols (such as dianhydrohexitols and cyclohexanediol), or oligomeric diols based on ethylene glycol (such as diethylene glycol, triethylene glycol, tetraethylene glycol, or poly(ethylene glycols)s). Dicarboxylic acids can be any aliphatic dicarboxylic acid, such as α,ω-dicarboxylic acids (i.e., non-branched), branched dicarboxylic acids, cyclic dicarboxylic acids (such as cyclohexanedicarboxylic acid).

In some aspects, the PEA polymers have the following Formula XXXII: 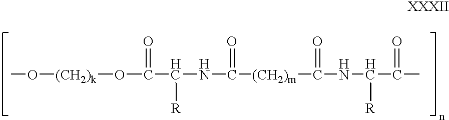
where k=2-12, especially 2, 3, 4 or 6; m=2-12, especially 4 or 8; and R=CH(CH₃)₂, CH₂CH(CH₃)₂, CH(CH₃)CH₂CH₃, (CH₂)₃CH₃, CH₂C₆H₅, or (CH₂)₃SCH₃.

In some embodiments, the second polymer comprises a mixture of a first PEA polymer in which A is L-phenylalanine (Phe-PEA) and a second PEA polymer in which A is L-leucine (Leu-PEA). The ration of Phe-PEA to Leu-PEA can be in the range of 10:1 to 1:1, or 5:1 to 2.5:1.

Optionally, the PEA polymer mixture includes an enzyme capable of hydrolytically cleaving the PEA polymer, such as α-chymotrypsin. The enzyme can be adsorbed on the surface of the biodegradable coated composition or can be included in bacteriophage that are released by action of the enzyme.

In accordance with the invention, the second polymer can be selected to compliment properties of the first polymer, such as fast bioactive agent release, relatively weak mechanical properties, and solvent solubility. In some aspects, acceptable second polymers can have slow bioactive agent release, indicating low diffusivities, which can be a function of higher glass transition temperatures, crystallinity, or specific chemical interactions with the bioactive agent.

Illustrative mechanical properties include flexibility and adhesion. For example, when the medical device to be coated with the inventive coatings is an expandable device (such as a stent), second polymers can be selected to be robust to device expansions. For example, polymers that may be considered robust can possess sufficient flexibility to accommodate device expansion, indicating that lower glass transition temperatures and lower crystallinity can be desirable. The second polymer can be selected to provide enhanced adhesion of the polymer coating to a device surface. In these aspects, unblended, single coatings of the first polymer can insufficiently adhere to a device surface. For example, PEGT/PBT does not typically adhere well to metal substrates. However, upon addition of additional coated layers composed of a different polymer in accordance with principles of the invention, such adhesion to the device surface can be enhanced.

In some aspects, the second polymer typically dissolves in the same solvents (such as chloroform, THF, dichloromethane, and trichloromethane) as the first polymer (such as PEGT/PBT).

In some aspects, one or more coated layers of the inventive biodegradable coatings can be composed of a blend of two or more polymers. Such blends can be composed of two or more polymers having different bioactive agent release rates, for example. In some embodiments, the blend comprises a first polymer (selected from those described as first polymers herein) and a second polymer (selected from those described as second polymers herein). In other embodiments, the blend comprises two or more polymers selected from those described as suitable for use as second polymers (for example, a blend of PLA and PLGA, or a blend of collagen and hyaluronic acid, and so on). The individual polymers of a particular blend can be chosen to provide a desired release rate of bioactive agent. For example, a faster releasing polymer (such as PolyActive™ polymer) can be blended with a relatively slower releasing polymer (such as PLLA) to provide a blended polymer that demonstrates a release rate that is intermediate to the release rates of either polymer (PolyActive™ polymer or PLLA) alone.

In some aspects, the individual polymers of the blend can be chosen based upon other features of the polymers, such as, for example, the chemical characteristics of the degradation products of each polymer. For example, a polymer having relatively acidic degradation products (such as PLLA) can be blended with another polymer having non-acidic degradation products. Such blends can be beneficial, for example, when the particular bioactive agent to be included in the coating is sensitive to acidic environments. By blending polymers chosen to reduce the acidity of degradation products, the acidity of the treatment site can be reduced, thereby increasing efficacy of the bioactive agent in the treatment site.

The first biodegradable polymer and second biodegradable polymer are provided in a multiple layer format, thereby forming a bioactive agent releasing coating. The bioactive agent can be present in the first polymer, the second polymer, or both (in other words, bioactive agent can be present in any one or more of the individual coated layers of a coating). In some aspects, the coated composition does not undergo significant phase separation under conditions of use (typically, the conditions of use will range from storage conditions to device usage temperatures). Typical storage temperatures will be ambient temperatures (or about 18° C. to about 30° C.), while typical usage temperatures will be body temperatures (or about 36° C. to about 38° C.). Once provided at a medical article surface, the individual coated layers can remain distinct, with little to no mixing of components between layers. As illustrated in the Examples herein, the invention can provide a multiple layer format wherein individual coated layer integrity is retained on the device surface, and bioactive agent is mixed well within the particular coated layer(s) selected to include bioactive agent.

Selection of the second polymer can be impacted by one or more considerations, such as, for example, the bioactive agent release rate desired for a particular application, the bioactive agent release rate of the individual polymer under consideration as a second polymer, the hydrophobicity of the individual polymer, and solvent compatibility. As an initial step, a bioactive agent is selected for treatment. Next a release rate that would provide a therapeutic dosage of the bioactive agent to a patient can be determined, based upon (for example) many of the considerations mentioned herein. Once a biodegradable composition release rate is determined, this rate can be utilized to establish parameters for selection of the second polymer. The bioactive agent release rate for the first polymer can be determined, as discussed herein. The bioactive agent release rate for the second polymer can be determined separately, for example, utilizing information provided by the supplier of the polymer. Typically, the biodegradable composition release rate will be a rate that is intermediate to the release rate of the first polymer alone and second polymer alone.

The relative amounts and dosage rates of bioactive agent delivered over time can be controlled by modulating any one or more of the following: selection of the second biodegradable polymer; adjustment of the amounts of faster releasing polymers relative to slower releasing polymers within the biodegradable compositions; placement of the biodegradable polymers as layers within the biodegradable composition (for example, placement at the outer surface of a coating versus a more interior position that is proximate to the device surface, or placement at an intermediate location, between inner and outer layers). For higher initial release rates, the proportion of faster releasing polymer can be increased relative to the slower releasing polymer. If most of the dosage is desired to be released over a long time period, the proportion of slower releasing polymer can be increased relative to the slower releasing polymer.

The relative hydrophobicity of the second polymer can impact release rate of the bioactive agent. For example, compositions composed of one or more coated layers of PEGT/PBT (which is a relatively hydrophilic copolymer), one or more layers of a more hydrophobic second polymer can be chosen to modulate the release profile of bioactive agent over time.

Another selection parameter for the second polymer is solvent compatibility. In some preferred aspects, the solvent system for the first polymer and second polymer are compatible. In further aspects, the solvent system for the first biodegradable polymer, second biodegradable polymer and bioactive agent are compatible. Further, the first polymer and second polymer can be formulated to provide a coating solution that is easily applied to a device surface. For example, when it is desirable to apply the coating solution to a device surface utilizing spray techniques, it can be useful to form a coating solution that provides good atomization for such application, without undergoing phase separation during the application process.

The principle mode of degradation for many of the biodegradable polymers (and particularly the lactide and glycolide polymers and copolymers) is hydrolysis. Degradation proceeds first by diffusion of water into the material followed by random hydrolysis, fragmentation of the material, and finally a more extensive hydrolysis accompanied by phagocytosis, diffusion, and metabolism. The hydrolysis can be affected by the size and hydrophilicity of the particular polymer material, the crystallinity of the polymer, and the pH and temperature of the environment.

Once the polymer is hydrolyzed, the products of hydrolysis are either metabolized or secreted. The lactic acid generated by the hydrolytic degradation of PLA becomes incorporated into the tricarboxylic acid cycle and is secreted as carbon dioxide and water. Poly(glycolic acid) (PGA) is also broken down by random hydrolysis accompanied by non-specific enzymatic hydrolysis to glycolic acid that is either secreted or enzymatically converted to other metabolized species.

In some aspects, degradation of PLA, PGA and the like can generate an acidic environment in proximity to the device. Such acidic conditions can adversely impact bioactive agent, biodegradable polymer, or both. In preferred aspects, the inventive biodegradable compositions are formulated such that the amount of acidic degradation products (such as those generated upon degradation of PLLA or PLGA) are controlled to reduce or minimize risk of damage to bioactive agent provided in the biodegradable compositions. Since many bioactive agents are acid-sensitive, it can be beneficial to provide biodegradable compositions that can reduce the amount of biodegradable polymer that could create an acidic environment upon degradation, and/or provide a protective environment for such bioactive agents.

In some aspects, the degradable composition includes a first coated layer composed of a PEGT/PBT polymer (commercially available from Octoplus BV, under the trade designation PolyActive™), and a second coated layer composed of PLLA. These embodiments deliver bioactive agent over a longer time period, and with a lower initial burst, relative to embodiments having a single coated layer composed of PolyActive™. The bioactive agent release is intermediate the release rates of PolyActive™ polymer alone and PLLA alone. The relative thicknesses of the first coated layer and second coated layer can be adjusted to achieve the desired combination of initial dosage rate and subsequent constant and longer lasting dosage rate.

The location and chemical composition of the coated layers can be selected to provide controlled release of a bioactive agent. As mentioned herein, the designation of a “first” coated layer, “second” coated layer, and so on, is not meant to limit the inventive compositions and methods to a particular sequence of coated layers on a surface. For purposes of describing the invention, however, the coated layers comprising the coating are so designated to indicate the distinct chemical composition of the coated layers.

The multilayer coatings include a first coated layer (which is typically the coated layer placed directly in contact with the implantable device surface), a second coated layer (which is typically the coated layer placed directly in contact with the first coated layer), and so on. In the case of a two-layer construction, then, the second coated layer can be considered the outermost coated layer. For three-layer constructions, the third coated layer can be considered the outermost coated layer. It is the outermost coated layer that initially comes in contact with bodily fluids upon implantation of the device. While not intending to be bound by a particular theory, it is believed that selection of the outermost coated layer on the device can impact biocompatibility and release rate of the biodegradable composition. In some preferred aspects, a hydrophilic polymer is selected as the outermost coated layer, and thus the layer that comes in contact with bodily fluids upon placement of the device in the body.

For example, the outermost coated layer can be selected to improve coating biocompatibility. In one such preferred embodiment, an outermost coated layer of PolyActive™ polymer can improve coating biocompatibility by presenting a surface that generates significantly less acid relative to PLLA, PDLLA, PLGA, or the like during degradation than the hydrophobic biodegradable polymers. As a result, at least the implantation site (and perhaps a larger area surrounding the implanted device) will be less acidic during degradation of the biodegradable composition.

Further, placement of a more hydrophilic outermost coated layer (such as PolyActive™ polymer) can also preferably increase the degradation rate of the coatings by allowing a greater rate of water penetration into the coatings.

Alternatively, when generation of acidic degradation products is not an issue, an outermost coated layer of PLLA (or similar polymer) can be provided. According to these embodiments, the outermost layer will have a bioactive agent release rate that is slower than the first coated layer. Thus, the outermost layer can act to slow release of the bioactive agent from the biodegradable composition. Moreover, the relatively hydrophobic nature of these types of polymers (such as PLLA) can reduce the water permeability of the biodegradable composition, thereby reducing the diffusion of bioactive agent from the biodegradable composition. This can impact the initial release of the bioactive agent.

Suitable solvents that can be used to formulate the biodegradable composition include, but are not limited to, chloroform, water, alcohol (including, for example, methyl, ethyl, isopropyl and the like), acetone, acetonitrile, ether, methyl ethyl ketone (MEK), ethyl acetate, tetrahydrofuran (THF), dioxane, methylene chloride, xylene, toluene, N,N-dimethylformamide (DMF), dimethyl sulfoxide (DMSO), N,N-dimethylacetamide (DMAC), N-methylpyrrolidone (NMP), dichloromethane, hexane, combinations of these, and the like.

To form biodegradable composition with bioactive agent, the selected biodegradable polymer is mixed with a bioactive agent. The bioactive agent can be present as a liquid, a finely divided solid, or any other appropriate physical form. The variety of different bioactive agents that can be used in conjunction with the polymers of the invention is vast. The inventive biodegradable compositions can find particular utility for delivery of small molecular weight bioactive agents, as described herein. Optionally, the biodegradable composition can include one or more additives, such as diluents, carriers, excipients, stabilizers, or the like.

Upon contact with body fluids, the biodegradable composition undergoes gradual degradation (mainly through hydrolysis) with concomitant release of the bioactive agent for a sustained or extended period. This can result in prolonged delivery (such as a period of several weeks) of therapeutically effective amounts of the bioactive agent. The therapeutically effective amount can be determined based upon such factors as the patient being treated, the severity of the condition, the judgment of the prescribing physician, and the like. In light of the teaching herein, those skilled in the art will be capable of preparing a variety of formulations.

The various figures illustrate the changes in elution rates possible with variation in coating materials and relative position of biodegradable polymers. The various examples herein illustrate the different elution rates possible for a coated layers of various compositions, various relative positions within the coating, various coating thicknesses, and the like.

Each coating system has its own release kinetics profile that can be adjusted by polymeric composition and relative position of individual polymer layers within the coating. Each biodegradable coating can consist of different polymers as well as being provided with different molecules (bioactive agents and other additives). This provides the ability to control release kinetics at each coated layer, and, in turn, the ability to manipulate dosage of one or more bioactive agents from the biodegradable composition.

The biodegradable compositions of the invention include one or more bioactive agents, thereby providing a drug-delivery device. These drug-delivery aspects will now be described in more detail.

As used herein, “bioactive agent” refers to an agent that affects physiology of biological tissue. Bioactive agents useful according to the invention include virtually any substance that possesses desirable therapeutic characteristics for application to the implantation site.

For ease of discussion, reference will repeatedly be made to a “bioactive agent.” While reference will be made to a “bioactive agent,” it will be understood that the invention can provide any number of bioactive agents to a treatment site. Thus, reference to the singular form of “bioactive agent” is intended to encompass the plural form as well. Moreover, for purposes of discussion, reference will be made to association of the bioactive agent with a biodegradable composition composed of blends of PEGT/PBT and a second polymer, such as PLA. However, it will be apparent upon review of this disclosure that the bioactive agent can be associated with any of the biodegradable polymeric compositions described herein. Further, the additives described herein are applicable to all polymer systems disclosed as well.

Exemplary bioactive agents include, but are not limited to, thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, antimicrobial agents, antibiotics, antibacterial agents, antiparasite and/or antiprotozoal solutes, antiseptics, antifungals, angiogenic agents, anti-angiogenic agents, inhibitors of surface glycoprotein receptors, antimitotics, microtubule inhibitors, antisecretory agents, actin inhibitors, remodeling inhibitors, antisense nucleotides, anti-metabolites, miotic agents, anti-proliferatives, anticancer chemotherapeutic agents, anti-neoplastic agents, antipolymerases, antivirals, anti-AIDS substances, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, extracellular matrix components, ACE inhibitors, free radical scavengers, chelators, anti-oxidants, photodynamic therapy agents, gene therapy agents, anesthetics, immunotoxins, neurotoxins, opioids, dopamine agonists, hypnotics, antihistamines, tranquilizers, anticonvulsants, muscle relaxants and anti-Parkinson substances, antispasmodics and muscle contractants, anticholinergics, ophthalmic agents, antiglaucoma solutes, prostaglandins, antidepressants, antipsychotic substances, neurotransmitters, anti-emetics, imaging agents, specific targeting agents, and cell response modifiers.

More specifically, in embodiments the active agent can include heparin, covalent heparin, synthetic heparin salts, or another thrombin inhibitor; hirudin, hirulog, argatroban, D-phenylalanyl-L-poly-L-arginyl chloromethyl ketone, or another antithrombogenic agent; urokinase, streptokinase, a tissue plasminogen activator, or another thrombolytic agent; a fibrinolytic agent; a vasospasm inhibitor; a calcium channel blocker, a nitrate, nitric oxide, a nitric oxide promoter, nitric oxide donors, dipyridamole, or another vasodilator; HYTRIN® or other antihypertensive agents; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; dimethyl sulfoxide (DMSO), a retinoid, or another antisecretory agent; cytochalasin or another actin inhibitor; cell cycle inhibitors; remodeling inhibitors; deoxyribonucleic acid, an antisense nucleotide, or another agent for molecular genetic intervention; methotrexate, or another antimetabolite or antiproliferative agent; tamoxifen citrate, TAXOL®, paclitaxel, or the derivatives thereof, rapamycin (or other rapalogs, e.g. ABT-578 or sirolimus), vinblastine, vincristine, vinorelbine, etoposide, tenopiside, dactinomycin (actinomycin D), daunorubicin, doxorubicin, idarubicin, anthracyclines, mitoxantrone, bleomycin, plicamycin (mithramycin), mitomycin, mechlorethamine, cyclophosphamide and its analogs, chlorambucil, ethylenimines, methylmelamines, alkyl sulfonates (e.g., busulfan), nitrosoureas (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, morpholino phosphorodiamidate oligomer or other anti-cancer chemotherapeutic agents; cyclosporin, tacrolimus (FK-506), pimecrolimus, azathioprine, mycophenolate mofetil, mTOR inhibitors, or another immunosuppressive agent; cortisol, cortisone, dexamethasone, dexamethasone sodium phosphate, dexamethasone acetate, dexamethasone derivatives, betamethasone, fludrocortisone, prednisone, prednisolone, 6U-methylprednisolone, triamcinolone (e.g., triamcinolone acetonide), or another steroidal agent; trapidil (a PDGF antagonist), angiopeptin (a growth hormone antagonist), angiogenin, a growth factor (such as vascular endothelial growth factor (VEGF)), or an anti-growth factor antibody (e.g., ranibizumab, which is sold under the tradename LUCENTIS®), or another growth factor antagonist or agonist; dopamine, bromocriptine mesylate, pergolide mesylate, or another dopamine agonist; ⁶⁰Co (5.3 year half life), ¹⁹²Ir (73.8 days), ³²P (14.3 days), ¹¹¹In (68 hours), ⁹⁰Y (64 hours), ⁹⁹Tc (6 hours), or another radiotherapeutic agent; iodine-containing compounds, barium-containing compounds, gold, tantalum, platinum, tungsten or another heavy metal functioning as a radiopaque agent; a peptide, a protein, an extracellular matrix component, a cellular component or another biologic agent; captopril, enalapril or another angiotensin converting enzyme (ACE) inhibitor; angiotensin receptor blockers; enzyme inhibitors (including growth factor signal transduction kinase inhibitors); ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³¹I-, ³²P- or ³⁶ S-radiolabelled form or other radiolabelled form of any of the foregoing; an estrogen (such as estradiol, estriol, estrone, and the like) or another sex hormone; AZT or other antipolymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluorozinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin, or other antibody targeted therapy agents; gene therapy agents; enalapril and other prodrugs; PROSCAR®, HYTRIN® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these.

Other biologically useful compounds that can also be included in the coating material include, but are not limited to, hormones, β-blockers, anti-anginal agents, cardiac inotropic agents, corticosteroids, analgesics, anti-inflammatory agents, anti-arrhythmic agents, immunosuppressants, anti-bacterial agents, anti-hypertensive agents, anti-malarials, anti-neoplastic agents, anti-protozoal agents, anti-thyroid agents, sedatives, hypnotics and neuroleptics, diuretics, anti-parkinsonian agents, gastro-intestinal agents, anti-viral agents, anti-diabetics, anti-epileptics, anti-fungal agents, histamine H-receptor antagonists, lipid regulating agents, muscle relaxants, nutritional agents such as vitamins and minerals, stimulants, nucleic acids, polypeptides, and vaccines.

Antibiotics are substances that inhibit the growth of or kill microorganisms. Antibiotics can be produced synthetically or by microorganisms. Examples of antibiotics include penicillin, tetracycline, chloramphenicol, minocycline, doxycycline, vancomycin, bacitracin, kanamycin, neomycin, gentamycin, erythromycin, geldanamycin, geldanamycin analogs, cephalosporins, or the like. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

Antiseptics are recognized as substances that prevent or arrest the growth or action of microorganisms, generally in a nonspecific fashion, e.g., either by inhibiting their activity or destroying them. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Antiviral agents are substances capable of destroying or suppressing the replication of viruses. Examples of anti-viral agents include α-methyl-1-adamantanemethylamine, hydroxy-ethoxymethylguanine, adamantanamine, 5-iodo-2′-deoxyuridine, trifluorothymidine, interferon, and adenine arabinoside.

Enzyme inhibitors are substances that inhibit an enzymatic reaction. Examples of enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(α-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylxanthne, papaverine HCl, indomethacind, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate R(+), p-aminoglutethimide tartrate S(−), 3-iodotyrosine, alpha-methyltyrosine L(−), alpha-methyltyrosine D(−), cetazolamide, dichlorphenamide, 6-hydroxy-2-benzothiazolesulfonamide, and allopurinol.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Local anesthetics are substances that have an anesthetic effect in a localized region. Examples of such anesthetics include procaine, lidocaine, tetracaine and dibucaine.

Imaging agents are agents capable of imaging a desired site, e.g., tumor, in vivo. Examples of imaging agents include substances having a label that is detectable in vivo, e.g., antibodies attached to fluorescent labels. The term antibody includes whole antibodies or fragments thereof.

Cell response modifiers are chemotactic factors such as platelet-derived growth factor (PDGF). Other chemotactic factors include neutrophil-activating protein, monocyte chemoattractant protein, macrophage-inflammatory protein, SIS (small inducible secreted), platelet factor, platelet basic protein, melanoma growth stimulating activity, epidermal growth factor, transforming growth factor alpha, fibroblast growth factor, platelet-derived endothelial cell growth factor, insulin-like growth factor, nerve growth factor, bone growth/cartilage-inducing factor (alpha and beta), and matrix metalloproteinase inhibitors. Other cell response modifiers are the interleukins, interleukin receptors, interleukin inhibitors, interferons, including alpha, beta, and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins, antisense molecules, androgenic receptor blockers and statin agents.

In an embodiment, the active agent can be in a microparticle. In an embodiment, microparticles can be dispersed on the surface of the substrate.

The weight of the coating attributable to the active agent can be in any range desired for a given active agent in a given application. In some embodiments, weight of the coating attributable to the active agent is in the range of about 1 microgram to about 10 milligrams of active agent per cm² of the effective surface area of the device. By “effective” surface area it is meant the surface amenable to being coated with the composition itself. For a flat, nonporous, surface, for instance, this will generally be the macroscopic surface area itself, while for considerably more porous or convoluted (e.g., corrugated, pleated, or fibrous) surfaces the effective surface area can be significantly greater than the corresponding macroscopic surface area. In an embodiment, the weight of the coating attributable to the active agent is between about 0.01 mg and about 0.5 mg of active agent per cm² of the gross surface area of the device. In an embodiment, the weight of the coating attributable to the active agent is greater than about 0.01 mg.

In some embodiments, more than one active agent can be used in the coating. Specifically, co-agents or co-drugs can be used. A co-agent or co-drug can act differently than the first agent or drug. The co-agent or co-drug can have an elution profile that is different than the first agent or drug.

In some embodiments, the active agent can be hydrophilic. In an embodiment, the active agent can have a molecular weight of less than 1500 daltons and can have a water solubility of greater than 10 mg/ml at 25° C. In some embodiments, the active agent can be hydrophobic. In an embodiment, the active agent can have a water solubility of less than 10 mg/ml at 25° C.

Biodegradable compositions can be formulated by mixing one or more bioactive agents with the polymers. The bioactive agent can be present as a liquid, a finely divided solid, or any other appropriate physical form. Typically, but optionally, the biodegradable composition will include one or more additives, such as diluents, carriers, excipients, stabilizers, or the like.

The particular bioactive agent, or combination of bioactive agents, can be selected depending upon one or more of the following factors: the application of the device (for example, coronary stent, orthopedic device, fixation element), the amount of the device composed of the polymeric material (for example, fabricating the entire device of polymeric material, versus providing the polymeric material as a coating on a device substrate), the medical condition to be treated, the anticipated duration of treatment, characteristics of the implantation site, the number and type of bioactive agents to be utilized, and the like.

The concentration of the bioactive agent in the biodegradable composition can be in the range of about 0.01% to about 75% by weight, or about 0.01% to about 50%, or about 1% to about 35%, or about 1% to about 20%, or about 1% to about 10% by weight, based on the weight of the final biodegradable composition. In some aspects, the bioactive agent is present in the biodegradable composition in an amount in the range of about 75% by weight or less, or about 50% by weight or less, or about 35% or less, or about 25% or less, or about 10% or less. The amount of bioactive agent in the biodegradable composition can be in the range of about 1 μg to about 10 mg, or about 100 μg to about 1000 μg, or about 300 μg to about 600 μg.

In some aspects, the bioactive agent should be stable in the selected solvent for the coating composition. For example, some organic solvents can adversely impact bioactive agent stability, particularly when the bioactive agent is present in the solvent over time. In some embodiments, bioactive agents such as rapamycin can be adversely impacted (e.g., degrade) over time when present in an aqueous solution. Thus, selection of solvent system for the coating compositions can be determined in part by consideration of the bioactive agent to be delivered from a medical article.

In one illustrative embodiment, when a relatively small-sized bioactive agent (for example, many antimicrobial agents, antiviral agents, and the like) is included in a PEGT/PBT polymeric material, the polyethylene glycol component of the copolymer preferably has a molecular weight in the range of about 200 to about 10,000, or about 300 to about 4,000. Also, the polyethylene glycol terephthalate is preferably present in the copolymer in an amount in the range of about 30 weight percent to about 80 weight percent of the weight of the copolymer, or in the range of about 50 weight percent to about 60 weight percent of the weight of the copolymer. According to these particular embodiments, the polybutylene terephthalate is present in the copolymer in an amount in the range of about 20 weight percent to about 70 weight percent of the copolymer, or in the range of about 40 weight percent to about 50 weight percent of the copolymer.

In some aspects, it can be desirable to provide one or more additives to the one or more of the polymers of the biodegradable composition. Such additives can be particularly desirable when bioactive agent is included in the polymer comprising the biodegradable composition. Additives can be included to impact the release of bioactive agent from the device. Suitable additives according to these aspects include, but are not limited to, hydrophobic antioxidants, hydrophobic molecules, and hydrophilic antioxidants, and excipients. Alternatively, additives can be included to impact imaging of the device once implanted. Illustrative additives will now be described in more detail. However, it is understood that such additives are optional; in some aspects, the inventive coating compositions do not require any additive to impact release of bioactive agent, since selection of first polymer and second polymer, as well as relative positioning of coated layers on a device surface, can achieve a wide variety of bioactive agent release rates without use of additives. Thus, in some embodiments, any additives utilized are useful for other features of the coated, besides the bioactive agent release rate.

In some embodiments, one or more of the polymers comprising the biodegradable composition can optionally include at least one hydrophobic antioxidant. For example, when the polyetherester material (such as PEGT/PBT) includes a hydrophobic small-sized drug (such as, for example, a steroid hormone), the polymer material can include at least one hydrophobic antioxidant. Exemplary hydrophobic antioxidants that can be employed include, but are not limited to, butylated hydroxytoluene (BHT), tocopherols (such as α-tocopherol, β-tocopherol, γ-tocopherol, δ-tocopherol, ε-tocopherol, zeta₁-tocopherol, zeta₂-tocopherol, and eta-tocopherol), and ascorbic acid 6-palmitate. Such hydrophobic antioxidants can retard the degradation of the polyetherester copolymer material, and can retard the release of the bioactive agent contained in the polymer. Thus, the use of a hydrophobic or lipophilic antioxidant can be desirable particularly to the formation of biodegradable compositions that include drugs that tend to be released quickly from the polymer, such as, for example, small drug molecules having a molecular weight less than 1500 (in other words, the use of a hydrophobic or lipophilic antioxidant can slow release of the drug from the biodegradable composition if desired). In some embodiments, the antioxidant can improve drug stability as well. For example, inclusion of rapamycin in drug eluting stents (“DES”) can be problematic, as rapamycin can be less stable than desired. Thus, inclusion of a hydrophobic antioxidant can, in some embodiments, improve the stability of rapamycin in a bioactive agent delivery device.

The hydrophobic antioxidant(s) can be present in the polymer in an amount in the range of about 0.01 weight percent to about 10 weight percent of the total weight of the polymer, or in the range of about 0.5 weight percent to about 2 weight percent.

In some embodiments, one or more polymers comprising the biodegradable composition can optionally include one or more hydrophobic molecules. For example, when the polyetherester material includes a hydrophilic small-size drug (for example an aminoglycoside such as gentamycin), the biodegradable composition can also include, in addition to or instead of the hydrophobic antioxidant herein described, at least one hydrophobic molecule such as cholesterol, ergosterol, lithocholic acid, cholic acid, dinosterol, betuline, and/or oleanolic acid. One or more hydrophobic molecules can act to retard the release rate of the bioactive agent from the polyetherester copolymer. Such hydrophobic molecules can prevent water penetration into the biodegradable composition, but do not compromise the degradability of the biodegradable composition. In addition, such molecules have melting points in the range of 150° C. to 200° C. or more. Therefore, a small percentage of these molecules increase the Tg of the polymer, which decreases the matrix diffusion coefficient for the bioactive agent to be released. Thus, such hydrophobic molecules can provide for a more sustained release of a bioactive agent from the biodegradable composition.

The hydrophobic molecule(s) can be present in the polymer in an amount in the range of about 0.1 weight percent to about 20 weight percent, or about 1 weight percent to about 5 weight percent, based upon the total weight of the polymer.

When the polyetherester copolymer contains a protein, the copolymer can also optionally include a hydrophilic antioxidant. Examples of hydrophilic antioxidants include, but are not limited to, those having the following structural formula XXXIII:
(X₁)_{Y}A-(X₂)_{Z} XXXIII
wherein each of Y and Z is 0 or 1, wherein at least one of Y and Z is 1. Each of X₁ and X₂ is independently selected from the group consisting of compounds of the formula XXXV: 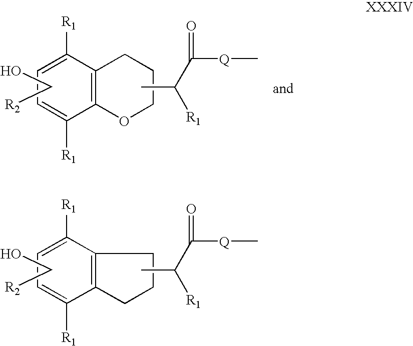
wherein each R₁ is hydrogen or an alkyl group having 1 to 4 carbon atoms, preferably methyl, and each R₁ is the same or different. R₂ is hydrogen or an alkyl group having 1 to 4 carbon atoms, preferably methyl. Q is NH or oxygen. Each of X₁ and X₂ can be the same or different. A is:
—(—R₃—O)ₙ—R₄ XXXV
wherein R₃ is an alkyl group having 1 or 2 carbon atoms, preferably 2 carbon atoms; n is 1 to 100, preferably from 4 to 22; R₄ is an alkyl group having 1 to 4 carbon atoms, preferably 1 or 2 carbon atoms.

In one embodiment, one of Y and Z is 1, and the other of Y and Z is 0. In another embodiment, each of Y and Z is 1.

In yet another embodiment, R₃ is ethyl.

In a further embodiment, R₄ is methyl or ethyl.

In yet another embodiment, R₁ is methyl, R₂ is methyl, R₃ is ethyl, R₄ is methyl, one of Y and Z is 1 and the other of Y and Z is 0, Q is NH, n is 21 or 22, and the antioxidant has the following structural formula XXXVI: 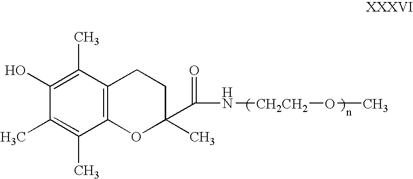
In another embodiment, the hydrophilic antioxidant has the following structural formula:
(X₃)_{Y}-A-(X₄)_{Z} XXXVII
wherein each of Y and Z is 0 or 1, wherein at least one of Y and Z is 1. Each of X₃ and X₄ is: 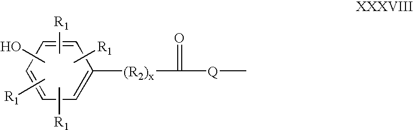
wherein each R₁ is hydrogen or an alkyl group having 1 to 4 carbon atoms, R₂ is an alkyl group having 1 to 4 carbon atoms, x is 0 or 1, and Q is NH or oxygen. Each R₁ is the same or different, and each of the X₃ and X₄ is the same or different. A is:
—(R₃—O—)ₙ—R₄ XXXIX
wherein R₃ is an alkyl group having 1 or 2 carbon atoms, preferably 2 carbon atoms; n is from 1 to 100, preferably from 4 to 22; and R₄ is an alkyl group having 1 to 4 carbon atoms, preferably 1 or 2 carbon atoms.

In one embodiment, at least one, preferably two, of the R₁ moieties is a tert-butyl moiety. When two of the R₁ moieties are tert-butyl moieties, each tert-butyl moiety is preferably adjacent to the —OH group.

The hydrophilic antioxidant(s) can be present in the polymer in an amount in the range of about 0.1 weight percent to about 10 weight percent, or about 1 weight percent to about 5 weight percent, based upon the total weight of the polymer.

As discussed herein, one or more of the polymers comprising the biodegradable composition can include a hydrophobic antioxidant, hydrophobic molecule, and/or a hydrophilic antioxidant in the amounts described herein. The type and precise amount of antioxidant or hydrophobic molecule employed can be dependent upon the molecular weight of the bioactive agent (protein), as well as properties of the polymer itself. If the polymer includes a large peptide or protein (such as, for example, insulin), the matrix can also optionally include a hydrophilic antioxidant such as those described herein and in the amounts described herein, and can also include polyethylene glycol having a molecular weight in the range of about 1,000 to about 4,000, in an amount in the range of about 1 weight percent to about 10 weight percent, based upon the total weight of the copolymer.

In some embodiments, one or more polymers comprising the biodegradable composition can further include imaging materials. For example, materials can be included in the biodegradable composition to assist in medical imaging of the device once implanted. Medical imaging materials are well known. Exemplary imaging materials include paramagnetic material, such as nanoparticular iron oxide, Gd, or Mn, a radioisotope, and non-toxic radio-opaque markers (for example, caged barium sulfate and bismuth trioxide). This can be useful for detection of medical devices that are implanted in the body (that are emplaced at the treatment site) or that travel through a portion of the body (that is, during implantation of the device). Paramagnetic resonance imaging, ultrasonic imaging, or other suitable detection techniques can detect such coated medical devices. In another example, microparticles that contain a vapor phase chemical can be used for ultrasonic imaging. Useful vapor phase chemicals include perfluorohydrocarbons, such as perfluoropentane and perfluorohexane, which are described in U.S. Pat. No. 5,558,854 (Issued 24 Sep., 1996); other vapor phase chemicals useful for ultrasonic imaging can be found in U.S. Pat. No. 6,261,537 (Issued 17 Jul., 2001).

In some aspects, one or more polymers comprising the biodegradable composition can include an excipient. A particular excipient can be selected based upon its melting point, solubility in a selected solvent (such as a solvent that dissolves the polymer and/or the bioactive agent), and the resulting characteristics of the composition. Excipients can comprises a few percent, about 5%, 10%, 15%, 20%, 25%, 30%, 40%, 50%, or higher percentage of the particular polymer in which it is included.

Buffers, acids, and bases can be incorporated in the polymer or polymers to adjust their pH. Agents to increase the diffusion distance of bioactive agents released from the polymer matrix can also be included. Illustrative excipients include salts, PEG or hydrophilic polymers, and acidic compounds.

Thus, additives can be included in one or more polymers comprising the biodegradable composition to assist in controlling release of bioactive agent, impacting degradation of the biodegradable composition, and/or impacting imaging of the device once implanted.

Release of bioactive agent can also be impacted by modification of one or more of the polymers comprising the biodegradable composition. Another technique for impacting release of bioactive agent can involve modifying the configuration of the device.

Optionally, the copolymer itself can be modified to affect the degradation rate and release rate of a bioactive agent. These aspects are particularly useful in embodiments comprising PEGT/PBT. For example, the copolymer can be modified by replacing components (monomeric units) with a particular hydrophobicity with a component (monomeric unit) that has a differing hydrophobicity.

In some embodiments, the configuration of the device can be manipulated to control release of the bioactive agent. For example, the surface area and/or size of the device can be manipulated to control dosage of the bioactive agent(s) provided to the implantation site.

The composition of the copolymer and/or the device configuration can be modified whether additives are included in the copolymer or not.

Preferably, the biodegradable composition is applied to selected surfaces of a medical device, such as a stent, wherein the stent itself is fabricated from a different material. The biodegradable composition coating can comprise a first polymer that is preferably a polyether ester copolymer, such as PEGT/PBT, and a second polymer selected as described herein. Other polymers that are suitable first biodegradable polymers are described herein.

In preferred aspects, the invention provides compositions and methods for providing biodegradable coatings containing bioactive agent to medical devices. The invention can be utilized in connection with medical devices having a variety of biomaterial surfaces. Illustrative biomaterials include metals and ceramics. The metals include, but are not limited to, titanium, Nitinol, stainless steel, tantalum, and cobalt chromium. A second class of metals includes the noble metals such as gold, silver, copper, and platinum iridium. Alloys of metals are suitable for biomaterials as well. The ceramics include, but are not limited to, silicon nitride, silicon carbide, zirconia, and alumina, as well as glass, silica, and sapphire.

Other illustrative biomaterials include those formed of synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, and vinylidene difluoride. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polylactic acid, polyglycolic acid, polydimethylsiloxanes, and polyetherketone.

Certain natural materials are also suitable biomaterials, including human tissue such as bone, cartilage, skin and teeth; and other organic materials such as wood, cellulose, compressed carbon, and rubber.

Combinations of ceramics and metals are another class of biomaterials. Another class of biomaterials is fibrous or porous in nature. The surface of such biomaterials can be pretreated (for example, with a Parylene coating composition) in order to alter the surface properties of the biomaterial, when desired.

The coatings of the invention are applied to a surface in a manner sufficient to provide a suitably durable and adherent coating on the surface. Typically, the coatings are provided in a manner such that they are not chemically bound to the surface. Rather, the coatings can be envisioned as encapsulating the device surface. Given the nature of the association between the coating and surface, it will be readily apparent that the coatings can be applied to virtually any surface material to provide a suitably durable and adherent coating. Moreover, in some embodiments, a suitable surface pretreatment can be utilized, to enhance the association between the coating and the device surface. For example, the device substrate surface may be roughened, or given a surface texture, by utilizing techniques (e.g. abrasion or micro-abrasion) well known in the art.

In some embodiments, the biodegradable composition is spray coated onto a surface of an implantable device, as described in the Examples herein. In other embodiments, the stent can be immersed in a biodegradable composition solution. Alternatively, the biodegradable composition can be extruded in the form of a tube that is then codrawn over a tube of stainless steel or Nitinol. By codrawing two tubes of the biodegradable composition over the metal tube, one positioned about the exterior of the metal tube and another positioned within such metal tube, a tube having multi-layered walls can be formed. Subsequent perforation of the tube walls to define a preselected pattern of spines and struts can impart the desired flexibility and expandability to the tube to create a stent.

The inventive biodegradable compositions can be applied to any desired portion of the device surface. For example, in some embodiments, the biodegradable composition coating can be provided on the entire surface of the device. In other embodiments, only a portion of the device can include the biodegradable composition coating. The portion of the device carrying the biodegradable composition coating can be selected based upon such factors as the application of the device, the amount of bioactive agent to be applied at a treatment site, the number and types of bioactive agents to be delivered, and like factors.

Moreover, each coated layer of the biodegradable composition can be provided on the surface of the device in any number of applications. The number of applications can be selected to provide individual coated layers of suitable thickness, as well as a desired total number of multiple coated layers of biodegradable composition, as desired. In such embodiments, the composition of individual layers of the coating can be the same or different, as desired. Typically, the thickness of the outermost coated layer is in the range of about 0.1 m to about 50 μm, or in the range of about 1 μm to about 10 μm. The thickness of the outermost coated layer can be selected based upon such factors as the chemical composition of the outermost coated layer (polymer selected, inclusion of bioactive agent, and the like). In some embodiments, the number of applications can be controlled to provide a desired overall thickness to the polymer coating. Generally, the thickness of the coating is selected so that it does not significantly increase the profile of the device for implantation and use within a patient. Typically, the overall thickness of the biodegradable composition coating is on the order of about 1 μm to about 100 μm.

The biodegradable composition can be applied as a multilayer coating on any device that is introduced temporarily or permanently into a mammal for the prophylaxis or therapy of a medical condition. These devices include any that are introduced subcutaneously, percutaneously, or surgically to rest within an organ, tissue, or lumen of an organ, such as arteries, veins, ventricles, or atrium of the heart.

Biodegradable compositions of the invention can be used to coat the surface of a variety of implantable devices, for example: drug-delivering vascular stents (e.g., self-expanding stents typically made from nitinol, balloon-expanded stents typically prepared from stainless steel); other vascular devices (e.g., grafts, catheters, valves, artificial hearts, heart assist devices); implantable defibrillators; blood oxygenator devices (e.g., tubing, membranes); surgical devices (e.g., sutures, staples, anastomosis devices, vertebral disks, bone pins, suture anchors, hemostatic barriers, clamps, screws, plates, clips, vascular implants, tissue adhesives and sealants, tissue scaffolds); membranes; cell culture devices; chromatographic support materials; biosensors; shunts for hydrocephalus; wound management devices; endoscopic devices; infection control devices; orthopedic devices (e.g., for joint implants, fracture repairs); dental devices (e.g., dental implants, fracture repair devices), urological devices (e.g., penile, sphincter, urethral, bladder and renal devices, and catheters); colostomy bag attachment devices; ophthalmic devices; glaucoma drain shunts; synthetic prostheses (e.g., breast); intraocular lenses; respiratory, peripheral cardiovascular, spinal, neurological, dental, ear/nose/throat (e.g., ear drainage tubes); renal devices; and dialysis (e.g., tubing, membranes, grafts).

Examples of useful devices include urinary catheters (e.g., surface-coated with antimicrobial agents such as vancomycin or norfloxacin), intravenous catheters (e.g., treated with antithrombotic agents (e.g., heparin, hirudin, coumadin), small diameter grafts, vascular grafts, artificial lung catheters, atrial septal defect closures, electro-stimulation leads for cardiac rhythm management (e.g., pacer leads), glucose sensors (long-term and short-term), degradable coronary stents (e.g., degradable, non-degradable, peripheral), blood pressure and stent graft catheters, birth control devices, benign prostate and prostate cancer implants, bone repair/augmentation devices, breast implants, cartilage repair devices, dental implants, implanted drug infusion tubes, intravitreal drug delivery devices, nerve regeneration conduits, oncological implants, electrostimulation leads, pain management implants, spinal/orthopedic repair devices, wound dressings, embolic protection filters, abdominal aortic aneurysm grafts, heart valves (e.g., mechanical, polymeric, tissue, percutaneous, carbon, sewing cuff), valve annuloplasty devices, mitral valve repair devices, vascular intervention devices, left ventricle assist devices, neuro aneurysm treatment coils, neurological catheters, left atrial appendage filters, hemodialysis devices, catheter cuff, anastomotic closures, vascular access catheters, cardiac sensors, uterine bleeding patches, urological catheters/stents/implants, in vitro diagnostics, aneurysm exclusion devices, and neuropatches.

Examples of other suitable devices include, but are not limited to, vena cava filters, urinary dialators, endoscopic surgical tissue extractors, atherectomy catheters, clot extraction catheters, percutaneous transluminal angioplasty catheters, PTCA catheters, stylets (vascular and non-vascular), coronary guidewires, drug infusion catheters, esophageal stents, circulatory support systems, angiographic catheters, transition sheaths and dialators, coronary and peripheral guidewires, hemodialysis catheters, neurovascular balloon catheters, tympanostomy vent tubes, cerebro-spinal fluid shunts, defibrillator leads, percutaneous closure devices, drainage tubes, thoracic cavity suction drainage catheters, electrophysiology catheters, stroke therapy catheters, abscess drainage catheters, biliary drainage products, dialysis catheters, central venous access catheters, and parental feeding catheters.

Examples of medical devices suitable for the present invention include, but are not limited to catheters, implantable vascular access ports, blood storage bags, vascular stents, blood tubing, arterial catheters, vascular grafts, intraaortic balloon pumps, cardiovascular sutures, total artificial hearts and ventricular assist pumps, extracorporeal devices such as blood oxygenators, blood filters, hemodialysis units, hemoperfusion units, plasmapheresis units, hybrid artificial organs such as pancreas or liver and artificial lungs, as well as filters adapted for deployment in a blood vessel in order to trap emboli (also known as “distal protection devices”).

In some aspects, the polymeric compositions can be utilized in connection with ophthalmic devices. Suitable ophthalmic devices in accordance with these aspects can provide bioactive agent to any desired area of the eye. In some aspects, the devices can be utilized to deliver bioactive agent to an anterior segment of the eye (in front of the lens), and/or a posterior segment of the eye (behind the lens). Suitable ophthalmic devices can also be utilized to provide bioactive agent to tissues in proximity to the eye, when desired.

In some aspects, the polymeric compositions can be utilized in connection with ophthalmic devices configured for placement at an external or internal site of the eye. Suitable external devices can be configured for topical administration of bioactive agent. Such external devices can reside on an external surface of the eye, such as the cornea (for example, contact lenses) or bulbar conjunctiva. In some embodiments, suitable external devices can reside in proximity to an external surface of the eye.

Devices configured for placement at an internal site of the eye can reside within any desired area of the eye. In some aspects, the ophthalmic devices can be configured for placement at an intraocular site, such as the vitreous. Illustrative intraocular devices include, but are not limited to, those described in U.S. Pat. No. 6,719,750 B2 (“Devices for Intraocular Drug Delivery,” Varner et al.) and U.S. Pat. No. 5,466,233 (“Tack for Intraocular Drug Delivery and Method for Inserting and Removing Same,” Weiner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and U.S. application Ser. No. 11/204,195 (filed Aug. 15, 2005, Anderson et al.), Ser. No. 11/204,271 (filed Aug. 15, 2005, Anderson et al.), Ser. No. 11/203,981 (filed Aug. 15, 2005, Anderson et al.), Ser. No. 11/203,879 (filed Aug. 15, 2005, Anderson et al.), Ser. No. 11/203,931 (filed Aug. 15, 2005, Anderson et al.), Ser. No. 11/225,301 (filed Sep. 12, 2005, Anderson et al.); and related applications.

In some aspects, the ophthalmic devices can be configured for placement at a subretinal area within the eye. Illustrative ophthalmic devices for subretinal application include, but are not limited to, those described in U.S. Patent Publication No. 2005/0143363 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); U.S. application Ser. No. 11/175,850 (“Methods and Devices for the Treatment of Ocular Conditions,” de Juan et al.); and related applications.

Suitable ophthalmic devices can be configured for placement within any desired tissues of the eye. For example, ophthalmic devices can be configured for placement at a subconjunctival area of the eye, such as devices positioned extrasclerally but under the conjunctiva, such as glaucoma drainage devices and the like.

The compositions are particularly useful for those devices that will come in contact with aqueous systems, such as bodily fluids. Such devices are coated with a coating composition adapted to release bioactive agent in a prolonged and controlled manner, generally beginning with the initial contact between the device surface and its aqueous environment. It is important to note that the local delivery of combinations of bioactive agents may be utilized to treat a wide variety of conditions utilizing any number of medical devices, or to enhance the function and/or life of the device. Essentially, any type of medical device may be coated in some fashion with one or more bioactive agents that enhances treatment over use of the singular use of the device or bioactive agent.

In one preferred embodiment, the coating composition can also be used to coat stents, e.g., either self-expanding stents, which are typically prepared from nitinol, or balloon-expandable stents, which are typically prepared from stainless steel. Other stent materials, such as cobalt chromium alloys, can be coated by the coating composition as well.

Devices which are particularly suitable include vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of suitable balloon-expandable stents are shown in U.S. Pat. No. 4,733,665 issued to Palmaz, U.S. Pat. No. 4,800,882 issued to Gianturco and U.S. Pat. No. 4,886,062 issued to Wiktor.

Optionally, the surface of some biomaterials can be pretreated (e.g., with a Parylene™ coating composition) in order to alter the surface properties of the biomaterial. Parylene C™ is the polymeric form of the low-molecular-weight dimer of para-chloro-xylylene. Supplied by Specialty Coating Systems (Indianapolis), a Parylene C™ coating can be deposited as a continuous coating on a variety of medical device parts to provide an evenly distributed, transparent coating. This deposition is accomplished by a process termed vapor deposition polymerization, in which dimeric Parylene C™ composition is vaporized under vacuum at 150° C., pyrolyzed at 680° C. to form a reactive monomer, then pumped into a chamber containing the component to be coated at 25° C. At the low chamber temperature, the monomeric xylylene is deposited on the part, where it immediately polymerizes via a free-radical process.

Deposition of the xylylene monomer takes place in only a moderate vacuum (0.1 torr) and is not line-of-sight. That is, the monomer has the opportunity to surround all sides of the part to be coated, penetrating into crevices or tubes and coating sharp points and edges, creating what is called a “conformal” coating. Other illustrative priming materials include silane, siloxane, polyurethane, polybutadiene, and polycarbodiimide.

In preferred aspects, the multiple layers that compose the biodegradable coating are applied sequentially and without intermediate curing or laminating steps. Typically, the individual polymer layers are simply dried between applications. Preferably, the coated layers adhere to the device surface and to each other without requiring any heating, pressure, or other treatment steps that could impact the stability of the bioactive agents and/or the polymer components of the coating. Surprisingly, the coated layers provide substantially durable coatings on device surfaces without requiring such treatments.

In use, the implantable device is placed within a patient at a desired implantation site. Upon contact with body fluids, the body fluids initially permeate at least a portion of the biodegradable composition, allowing for dissolution and diffusion of the bioactive agent from the biodegradable composition. The biodegradable composition undergoes gradual degradation (usually primarily through hydrolysis) with concomitant release of the dispersed bioactive agent for a sustained or extended period. This can result in prolonged delivery of therapeutically effective amounts of the bioactive agent.

In preferred aspects, the biodegradable composition includes polymers that are surface erodible and bulk erodible biodegradable materials. Surface erodible materials are materials in which bulk mass is lost primarily at the surface of the material that is in direct contact with the physiologic environment, such as body fluids. Bulk erodible materials are materials in which bulk mass is lost throughout the mass of the material; in other words, loss of bulk mass is not limited to mass loss that occurs primarily at the surface of the material in direct contact with the physiological environment.

In preferred aspects, the biodegradable composition is composed of only biodegradable polymers. In other words, the components of the biodegradable composition are selected to be broken down by the body over time.

Typically, current drug-eluting stents release anti-restenosis agent over a period of four (4) or more weeks. In preferred aspects, the inventive biodegradable compositions can provide a controlled release of bioactive agent to thereby provide a therapeutically effective dose of the bioactive agent for a sufficient time to provide the intended benefits. The controlled release includes both an initial release and subsequent sustained-release of the bioactive agent.

In preferred aspects, the inventive biodegradable compositions provide coatings that demonstrate excellent uniformity and durability during use. Coating uniformity and durability can be observed and assessed as follows.

One aspect of coating uniformity relates to surface features of the coating. The inventive coatings can be examined for uniformity and defects using a Field Emission Scanning Electron Microscope (SEM) at a low beam voltage (1 kV) which allows detailed imaging of surface features. Illustrative surface defects can include areas of delamination or cracking of the coating, surface areas that lack one or more coated layers, and the like. An overall survey of the coating quality is made at low magnification, and when features of interest are identified, higher magnification images are taken. From the overall survey, a qualitative ranking of the relative amount and type of defects in the coatings can be made.

Another aspect of coating uniformity relates to the uniformity of mixing of bioactive agent into the biodegradable compositions. This aspect of the coatings can be imaged using a confocal scanning Raman microscope. Laser light (532 nm wavelength) is focused onto the coating via a 100× microscope objective (numerical aperture 0.95), and the coating is scanned in three directions using a piezoelectric transducer driven platter. The scattered light from the coating is collected by the microscope, filtered, split into its spectrum using a spectrograph, and detected with a CCD detector. Thus, for each position (pixel) in the image, a Raman spectrum is measured. Reference spectra of the pure bioactive agent and pure polymer are incorporated into an augmented classical least squares analysis to create separate images of bioactive agent only and polymer only. These images are overlapped to create a composite color coded image of the distribution of bioactive agent within the polymer.

Uniformity of bioactive agent distribution within the coatings can impact the release profile of the bioactive agent. If a large percentage of the bioactive agent is concentrated at a particular portion of the coating, the release of the bioactive agent is less likely to exhibit controlled release kinetics. For example, if a large percentage of bioactive agent is concentrated at the surface of a coated layer, the bioactive agent is more likely to be released quickly from the coated layer, since the bioactive agent does not have a large diffusion distance to the surface. In contrast, a bioactive agent that is concentrated towards the device surface may have a larger diffusion distance to travel, and thus release of the bioactive agent may be delayed relative to the prior exemplary coating. Moreover, concentration of a bioactive agent within a coating can result in a release profile that includes one or more sudden increases in release, as polymer degradation reaches the area of bioactive agent concentration.

As used herein, the term “durability” refers to the ability of a coating to adhere to a device surface when subjected to forces typically encountered during use (for example, normal force, shear force, and the like). A more durable coating is less easily removed from a substrate by abrasion or compression. Durability of a coating can be assessed by subjecting the device to conditions that simulate use conditions. To simulate use of the coated devices, the coated stents are placed over sample angioplasty balloons. The stent is then crimped onto the balloon using a laboratory test crimper (available from Machine Solutions, Brooklyn, N.Y.). The stent and balloon are then placed in a water bath having a temperature of 37° C. After 5 minutes of soaking, the balloon is expanded using air at 5 atmospheres (3800 torr) of pressure. The balloon is then deflated, and the stent is removed. The stent is then examined by optical and scanning electron microscopy to determine the amount of coating damage caused by cracking and/or delamination. Herein, this durability testing will be referred to as the “Mechanical Testing.” Coatings with extensive damage are considered unacceptable for a commercial medical device. Testing can be followed up with contact angle testing, staining in Toluidine Blue solution (Aldrich, Milwaukee, Wis.), and/or SEM analysis to visualize the coating adherence to the substrate.

For purposes of illustrating the inventive concepts herein, the present discussion has focussed on providing the biodegradable compositions in the form of a coating on a surface of a device. However, given the present description, one of skill in the relevant art would readily appreciate that the biodegradable compositions can be utilized to form a structural component of the device itself. In these aspects, then, any selected component of the device structure can be fabricated of the biodegradable compositions of the invention, as desired.

The invention will now be described with reference to the following non-limiting examples.

### EXAMPLES

The following procedures and materials were used for the Examples.

For the examples, three multiblock copolymers of poly(ethylene glycol)terephthalate/poly(1,4-butylene)terephthalate (PEGT/PBT) were obtained from OctoPlus, B.V. Leiden, The Netherlands. These polymers are referred to as PolyActive™ and had the following properties:

| PEGT/PBT wt. | PEG average molecular weight (g/ | |
|---|---|---|
| ratio | mol) | Nomenclature |
| 55/45 | 300 | 300PEGT55PBT45 |
| 80/20 | 1000 | 1000PEGT80PBT20 |
| 55/45 | 1000 | 1000PEGT55PBT45 |

Poly(L-Lactide) with a weight-average molecular weight 100,000-150,000 and an inherent viscosity of 0.90-1.20 dL/g was used without further purification. Poly(DL-Lactide) with a weight-average molecular weight 75,000-120,000 and an inherent viscosity of 0.55-0.75 dL/g was used without further purification. Poly(DL-Lactide-co-Glycolide) with a weight-average molecular weight 50,000-75,000 and a composition of 50 mole percent of each monomer was used without further purification. These polymers are referred to as PLLA, PDLLA, and PLGA, respectively. All three polymers were purchased from Sigma-Aldrich (St. Louis, USA).

Poly(L-lactide-co-caprolactone-co-glycolide) [P(LLA-CL-GLA)] was obtained from Sigma-Aldrich (St. Louis, USA; Product No. 568562, average M_{w} approximately 100,000 by GPC, L-lactide 70%).

Poly[(lactide-co-ethyleneglycol)-co-ethyloxyphosphate] was obtained from Sigma-Aldrich (St. Louis, USA; Product No. 659606).

Dexamethasone (“Dexa”) was purchased from Sigma Aldrich (St. Louis, USA) and was 98% pure.

Paclitaxel (“PTX”) was purchased from LC Laboratories, a division of PKC Pharmaceuticals, Inc. (Woburn, Mass.) and was greater than 99% pure.

Surface Pretreatment by Application of Parylene C™ Coating

For all of the Examples, stents were first provided with a priming coated layer of Parylene C™. The Parylene C™ coating was accomplished by a process termed vapor deposition polymerization, in which dimeric Parylene C™ composition was vaporized under vacuum at 150° C., pyrolyzed at 680° C. to form a reactive monomer, then pumped into a chamber containing the component to be coated at 25° C. At the low chamber temperature, the monomeric xylylene was deposited on the part, where it immediately polymerized via a free-radical process. The polymer coating reached molecular weights of approximately 500 kilodaltons.

Deposition of the xylylene monomer took place in only a moderate vacuum (0.1 torr) and was not line-of-sight. That is, the monomer had the opportunity to surround all sides of the part to be coated, penetrating into crevices or tubes and coating sharp points and edges, creating what is called a “conformal” coating.

Preparation of Coated Layers Containing PolyActive™ Polymer

For preparation of polymer coating compositions including dexamethasone, the dexamethasone was first dissolved in THF and then added to a polymer/chloroform solution. Each PolyActive™ polymer was dissolved into chloroform with dexamethasone or paclitaxel. The concentration of PolyActive™ polymer was 27 milligram per milliliter while concentration of dexamethasone or paclitaxel was 3 milligram per milliliter. The resulting solution was agitated at 25° C. until there was no evidence by visible inspection of insoluble material.

Preparation of PLLA, PDLLA, or PLGA Coatings

For preparation of polymer coating compositions including dexamethasone, the dexamethasone was first dissolved in THF and then added to a polymer/chloroform solution. Each PLLA, PDLLA, and PLGA polymer was dissolved into chloroform with dexamethasone or paclitaxel. The concentration of polymer was 27 milligram per milliliter while the concentration of dexamethasone or paclitaxel was 3 milligram per milliliter. The resulting solution was agitated at 25° C. until there was no evidence by visible inspection of insoluble material.

Coating Procedure

Each coating solution was applied to commercially available stainless steel stents (for example Laserage Technology Corporation, IL) using an ultrasonic spray head connected to a syringe pump. See U.S. Patent Application Publication No. US 2004/0062875 A1 (Chappa et al., “Advance Coating Apparatus and Method,” Apr. 1, 2004). After coating, the stents were placed under vacuum to remove the solvent. Typical coating weights on each stent were approximately 500 micrograms after drying, unless indicated specifically to the contrary.

Bioactive Agent Elution Experiments

The following elution experiments were utilized for coatings containing dexamethasone. Before and after stent coating, each stent was weighed to measure the amount of coating on the stent. Bioactive agent release was measured in phosphate-buffered saline (PBS, pH 7.4) or 0.45% Tween Acetate Buffer (TAB, in distilled water). In a typical procedure, each stent was placed in a 5-milliliter amber scintillation vial. A magnetic stir bar and 4 milliliters of PBS buffer (1 liter water, 9 grams sodium chloride, 0.27 grams potassium phosphate monobasic (KH₂PO₄), and 1.4 grams potassium phosphate dibasic (K₂HPO₄)) was added to each of the vials. The vials were placed in a 37° C. water bath. At each sampling time (usually 4 or 5 times on the first day followed by daily sampling thereafter), the stent was removed and placed in fresh buffer solution in a new vial. Concentration of bioactive agent (dexamethasone) at each sampling time was determined in the spent buffer by UV spectroscopy using the characteristic wavelength for each bioactive agent. This concentration was converted to a mass of bioactive agent released from the coating using molar absorptivities.

For assays utilizing TAB, 4 milliliters of TAB buffer (1 liter water, 0.704 g sodium acetate, and 1.6 ml 1 M acetic acid, and 4.05 ml Tween 80) was added to each of the vials. The vials were placed in a 37° C. water bath. At each sampling time, the stent was removed and placed in fresh buffer solution in a new vial, as described for the PBS elution assay. Concentration of bioactive agent at each sampling time was determined in the spent buffer by HPLC.

The cumulative mass of the released bioactive agent was calculated by adding the individual sample mass after each removal. The release profile was obtained by plotting the amount of released bioactive agent as a function of time.

Once the elution experiment was finished, the stents were dried overnight in a vacuum oven set at room temperature (25-27° C.) and weighed to ensure the accuracy of the UV spectroscopy results.

For coatings containing paclitaxel, the following procedures were followed to observe bioactive agent elution. Paclitaxel content and quality from coated stent samples were analyzed by immersing the paclitaxel coated stents into a glass test tube filled with 2.5 to 4 ml 0.1% acetic acid in MeOH, which dissolves coated material, including paclitaxel, from the cobalt chromium stent surface. The tube was capped, covered with aluminum foil and shaken for 3 hours using a mechanical shaker. After shaking, the solution was filtered via a 0.45 micron Nylon Acrodisc syringe filter (having the extracted paclitaxel) and was analyzed by HPLC using the following parameters:

HPLC column=ODS Hypersil C18, 150×4.6 mm, 5 u particle size

Column temp=35 deg C.

Mobile phase=50:50 acetonitrile/water

Flow rate=1.2 ml/min

Injection volume=10 ul

Rinse solution=80:20 acetonitrile/water

UV Detection wavelength=227 nm

Run time=10 min

The HPLC column was equilibrated with the mobile phase solution (50:50 acetonitrile/water) and tested using a paclitaxel standard, which produces a peak for paclitaxel at 227 nm. In order to determine the amount of paclitaxel (μg) content in the stent coating, 3 paclitaxel standard solutions were run in duplicate. The average peak area of each standard was used to generate a calibration curve (Peak area vs concentration in μg/ml). Next, test samples (0.1% AA/MeOH with paclitaxel extracted from coated stents) were run on the HPLC. The PTX concentration (in μg/ml) was determined from the standard curve and multiplied by the volume of 0.1% AA/MeOH to determine amount (in μg) of paclitaxel extracted from the coated stents.

### Example 1

### Elution of Bioactive Agent from Representative Multilayer Coatings Including Two Coated Layers

Various biodegradable coatings were prepared to include a representative small molecular weight bioactive agent, and the resultant elution profiles were observed.

For baseline comparisons, two groups of stents were provided with a single coated layer containing the bioactive agent. The first group of stents was provided with a single coated layer of PLLA and dexamethasone (Coating A), the coating composition prepared as described above. The second group of stents was provided with a single coated layer of PolyActive™ polymer and dexamethasone (Coating B), the coating composition prepared as described above.

In addition, a group of stents were provided with a second coated layer composed of PolyActive™ polymer (without addition of a bioactive agent) (Coating C). For these coated layers containing PolyActive™ polymer, the PolyActive™ polymer was dissolved in chloroform to a concentration of 20 milligrams per milliliter, and the resulting coating solution was applied over the first coated layer by ultrasonic spraying as described previously.

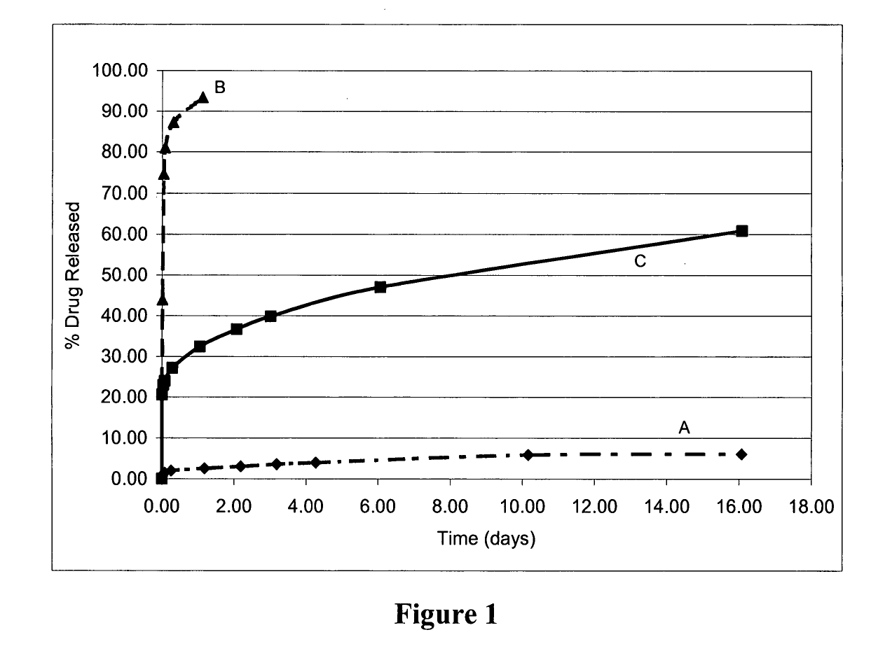

The various coated layers were applied to the stainless steel stents as previously described. The stents were then dried in a vacuum oven set at room temperature for 3 hours. Table 2 lists the coating and bioactive agent weights. Dexamethasone elution results from the coatings in Table 1 are shown in FIG. 1.

TABLE 2
Coating Characteristics

| | | | | Second | |
|---|---|---|---|---|---|
| | | | | Layer | Dexa |
| | First Coated | First Layer | Second Coated | Weight | Weight |
| Coating | Layer | Weight (μg) | Layer | (μg) | (μg) |
| A | PLLA/Dexa | 486 | None | N/A | 54 |
| B | 1000PEGT80PBT20/ | 521 | None | N/A | 52 |
| | Dexa | | | | |
| C | PLLA/Dexa | 280 | 1000PEGT80PBT20 | 285 | 92 |

Results indicated the individual polymers (unblended) exhibited set release rates. Stent coatings containing a single coated layer of PolyActive™ polymer released dexamethasone quickly due to the hydrophilic portions of the polymer allowing water penetration and rapid bioactive agent diffusion, Coating B. Stent coatings containing a single coated layer of PLLA released dexamethasone very slowly due to the PLLA hydrophobicity, Coating A. By creating a multi-layer coating composition, Coating C, the release rate was brought between these extremes. Coatings B and C demonstrated a steep initial release due to the dissolution of dexamethasone through the biodegradable composition. For Coating C, the steep initial release was followed by a sustained release controlled by the multilayer configuration of the biodegradable composition. The sustained release for Coating C was observed for 16 days.

The observed release profiles illustrated in FIG. 1 can be described as follows. Dexamethasone has a relatively low molecular weight (MW=392) and thus diffuses through a polymer matrix more easily than larger molecular weight bioactive agents. Release of dexamethasone from a single coated layer composed of PolyActive™ polymer (Coating B) showed a substantial burst (greater than 90% of bioactive agent) of bioactive agent within the first day. In contrast, coatings composed of a single coated layer composed of PLLA released dexamethasone much more slowly due to the hydrophobicity of PLLA (Coating A). No burst release was observed with the single coated layer of PLLA. For the multilayer coating, an initial release of approximately 30% of the dexamethasone was observed in the first day, followed by a substantially controlled release of the bioactive agent subsequent to the initial release. Thus, these experiments demonstrate the ability to control the initial burst of a relatively small molecular weight bioactive agent from biodegradable coating compositions.

FIG. 1 also shows the cumulative percentage of released dexamethasone over time for the three different biodegradable coatings. Compared to coatings containing a single coated layer of PolyActive™ polymer with dexamethasone, the multilayer coatings clearly demonstrate sustained-release kinetics. For example, the time for the release of 50% dexamethasone (t_{1/2}) is 8 days for the multilayer coating. In comparison, over 90% of the dexamethasone was released in the first day from the single layer PolyActive™ polymer formulation. Further, at day 16, the single layer PLLA coating released less than 10% of the dexamethasone.

Given the duration of the experiments (approximately 16 days), release of dexamethasone was primarily due to diffusion of the bioactive agent through the polymer matrix, and not by degradation of the polymer matrix.

### Example 2

### Elution of Bioactive Agent from Representative Multilayer Coatings Including Three Coated Layers and PolyActive™ Polymer Outer Coating

Stainless steel stents were provided with a coating composed of three coated layers, wherein the first coated layer included a model small molecular weight bioactive agent, dexamethasone. The coatings were evaluated for bioactive agent release as follows.

A first coated layer composed of PLLA and dexamethasone was prepared and applied to the stents as previously described. A second coated layer composed of PLLA (without bioactive agent) was prepared and applied to the stents as previously described. A third coated layer composed of PolyActive™ polymer (without bioactive agent) was prepared and applied to the stents as previously described. The average weight of the third coated layer for the stents was 130 micrograms. Table 3 lists the coating weights and composition for the first two coated layers.

TABLE 3
Coating Characteristics

| | | First | | Second | |
|---|---|---|---|---|---|
| | | Layer | | Layer | |
| | First Coated | Weight | Second Coated | Weight | Dexa |
| Coating | Layer | (μg) | Layer | (μg) | Weight (μg) |
| D | PLLA/Dexa | 486 | PLLA | 28 | 175 |
| E | PLLA/Dexa | 521 | PLLA | 60 | 170 |
| F | PLLA/Dexa | 280 | PLLA | 130 | 130 |

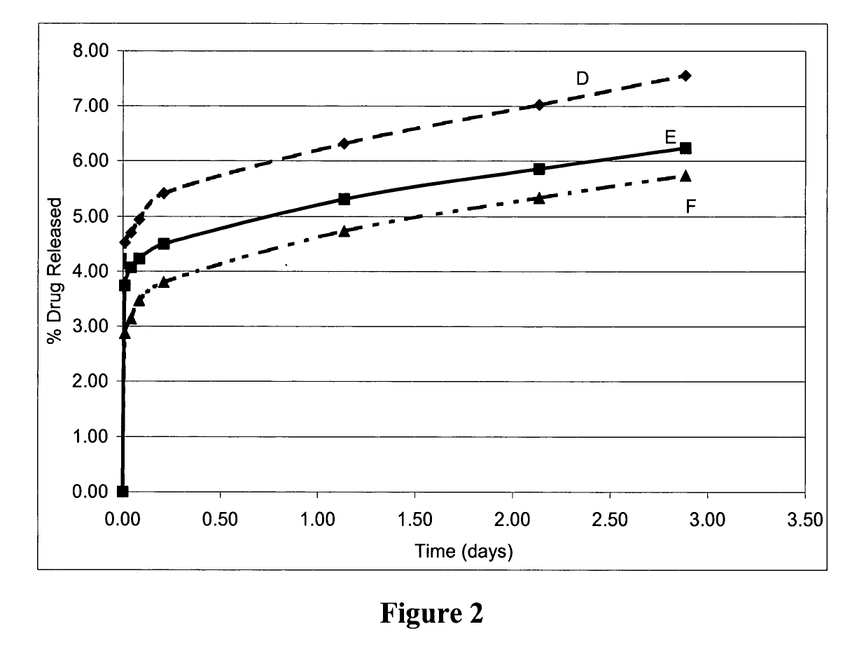

In FIG. 2, results indicated that these multi-layer coatings demonstrated a much lower initial release than coatings that did not include the bioactive agent-free third coated layer. Dexamethasone elution was controlled with the amount of bioactive agent-free second coated layer applied. Release control was observed for the initial release of the bioactive agent, as well as the subsequent release (that is, beginning at 0.25 Day and continuing thereafter at a relatively constant release rate). The thicker the second coated layer, the slower the bioactive agent eluted from the coating. All Coatings D-F demonstrated a dual phase release, including a steep initial release due to the dissolution of dexamethasone through the biodegradable composition followed by a sustained release controlled by the multilayer configuration of the biodegradable composition. The coatings demonstrated different burst doses, but similar sustained-release rates. The coatings shared the same multilayer configuration, but the coated layer thickness for the first coated layer and second coated layer was varied.

In addition to controlling the elution of a bioactive agent, the third coated layer (PolyActive™ polymer) can preferably improve coating biocompatibility by presenting a surface that generates significantly less acid relative to PLLA, PDLLA, PLGA, or the like during degradation than the hydrophobic biodegradable polymers. The hydrophilic third coated layer can also increase the degradation rate of the coatings by allowing a greater rate of water penetration into the coatings. These results are illustrated in FIG. 2.

The observed release profiles illustrated in FIG. 2 can be described as follows. For each of the coating compositions, the weight of the first polymer layer and second polymer layer were adjusted. All coatings included a third, outermost coated layer composed of PolyActive™ polymer, with an average coated layer weight of 130 μg. Coating D included the least amount of the second coated layer. Release of dexamethasone from Coating D showed the highest initial release (albeit still much less than 10% of the bioactive agent contained within the coating composition) within the first approximately six hours. In contrast, Coating F included the least amount of first coated layer and the highest amount of second coated layer. The resulting initial release and subsequent sustained release rate were markedly lower than Coating D. Coating E, which included the thickest first coated layer and an intermediate second coated layer, exhibited an initial release rate and subsequent release rate intermediate to the other coatings.

Results indicate that modification of the intermediate PLLA layer (second coated layer) can control the initial release rate and subsequent release rate of a relatively small molecular weight bioactive agent from biodegradable coating compositions. Thus, both the initial release rate and subsequent sustained release rate can be precisely controlled by adjusting the relative coating weights of the first and second coated layers.

FIG. 2 also shows the cumulative percentage of released dexamethasone over time for the three different biodegradable coatings. All coatings clearly demonstrate controlled initial release of bioactive agent, as well as sustained-release kinetics.

The release rate can be varied by using different loadings of the bioactive agent in the first coated layer, the bioactive agent free layers of PLLA type polymers, and/or different layer thicknesses of PolyActive™ polymers.

### Example 3

### Elution of Bioactive Agent from Representative Multilayer Coatings Including PLLA Outer Layer

Experiments were conducted to illustrate the effect of multiple coated layers on bioactive agent release profiles.

Stents were provided with a first coated layer containing either PolyActive™ polymer or PLLA with paclitaxel as a model bioactive agent. These coatings were prepared and applied to the stainless steel stents as described previously. For one group of stents (Stent K), a second coated layer of bioactive-agent free PLLA was applied to adjust bioactive agent release rate. For these coated layers, PLLA was dissolved in tetrahydrofuran to a concentration of 20 milligrams per milliliter, and then applied as a second layer over the existing bioactive agent containing coating by ultrasonic spraying. The stents were then dried in a vacuum oven set at room temperature.

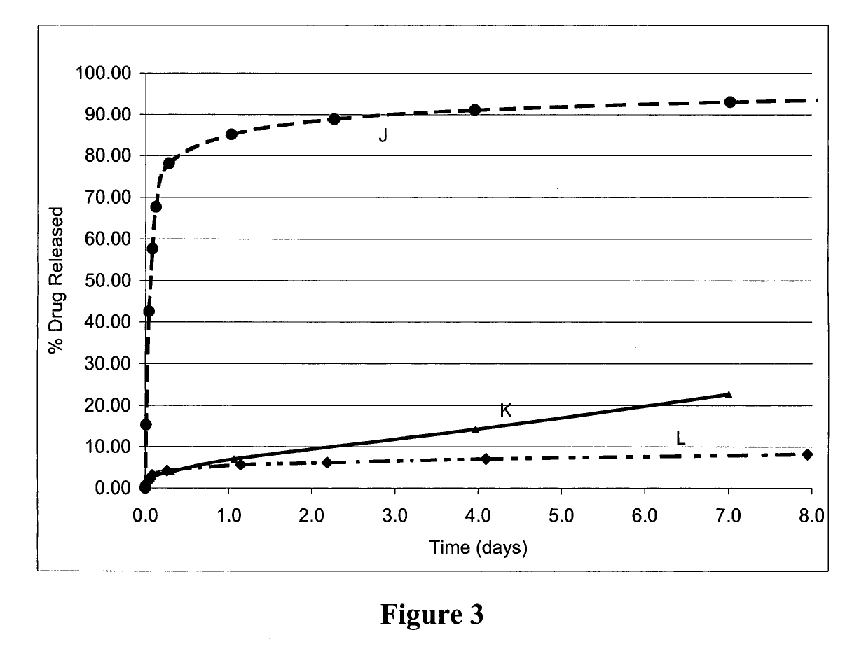

Table 4 lists the coating compositions and bioactive agent weights. FIG. 3 displays the paclitaxel elution results. The outer coated layer of PLLA slowed the elution to a rate between the polymer extremes including a lower bioactive agent burst than shown by the PolyActive™ polymer bioactive agent containing layer alone.

TABLE 4
Coating Characteristics

| | | First | | | |
|---|---|---|---|---|---|
| | | Layer | Second | Second | PTX |
| | | Weight | Coated | Layer | Weight |
| Coating | First Coated layer | (μg) | layer | Weight (μg) | (μg) |
| J | 300PEGT55PBT45/ | 560 | N/A | N/A | 56 |
| | PTX | | | | |
| K | 300PEGT55PBT45/ | 585 | PLLA | 110 | 58 |
| | PTX | | | | |
| L | PLLA/PTX | 575 | N/A | N/A | 56 |

As shown in FIG. 3, the initial bioactive agent release and subsequent release rate can be controlled by providing a second coated layer composed of PLLA. Coating J included a single layer of PolyActive™ polymer containing paclitaxel, and Coating L included a single layer of PLLA containing paclitaxel. These two single layer coatings lacked the second polymer coating of PLLA. For Coating K, the presence of a second coated layer of PLLA reduced the initial release of paclitaxel and provided a sustained release rate of the bioactive agent.

FIG. 3 also shows the cumulative percentage of released paclitaxel over time for the three different biodegradable coating compositions. For the single layer coating composed of PolyActive™ polymer and paclitaxel, approximately 85% of the bioactive agent was released within the first day. In contrast, the total amount of paclitaxel released from the single layer coating composed of PLLA was less than 10% at 8 days. The multilayer coating composed of a first coated layer including PolyActive™ polymer and paclitaxel, and a second coated layer of PLLA (no bioactive agent) exhibited an intermediate, controlled release profile that approximated zero order kinetics. Initial release of the paclitaxel (in the first day) was less than 10%, and at 7 days, a total of slightly more than 20% of the bioactive agent was released.

Results indicate that inclusion of a second coated layer composed of PLLA can control the initial bioactive agent release rate and subsequent release rate of a relatively small molecular weight bioactive agent from biodegradable coating compositions. Thus, both the initial burst release and subsequent sustained release rate (approximating zero-order release) can be precisely controlled by adjusting the relative coating weights of the first and second coated layers.

### Example 4

### Elution of Bioactive Agent from Representative Multilayer Coatings Including PLLA Outer Layer of Varying Weights

For these experiments, multiple layer coatings were prepared and applied to stents, wherein the amount of PLLA coated layer (not containing bioactive agent) was varied to show the effect of layer thickness on bioactive agent release rates.

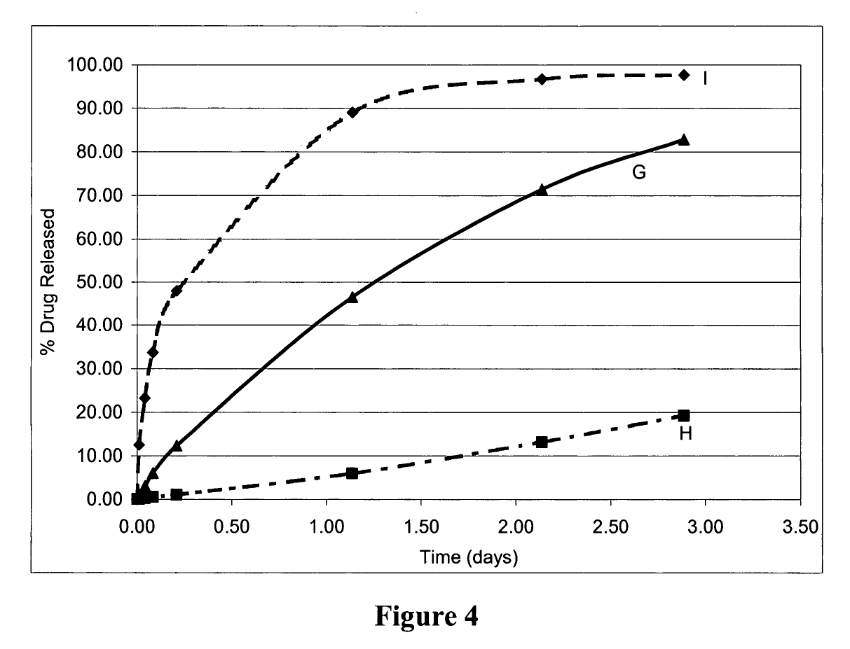

First coated layers containing PolyActive™ polymer and dexamethasone were prepared as previously described and applied to the stents as described previously. A second coated layer composed of PLLA along (no bioactive agent) was prepared as previously described and applied to the first coated layer as previously described. Table 5 lists the coating compositions and weights for this study. Elution results for these coatings are shown in FIG. 4.

TABLE 5
Coating Characteristics

| | | First | | Second | |
|---|---|---|---|---|---|
| | | Layer | Second | Layer | |
| | First Coated | Weight | Coated | Weight | Dexa |
| Coating | layer | (μg) | layer | (μg) | Weight (μg) |
| G | 300PEGT55PBT45/ | 530 | PLLA | 97 | 175 |
| | Dexa | | | | |
| H | 300PEGT55PBT45/ | 500 | PLLA | 210 | 162 |
| | Dexa | | | | |
| I | 300PEGT55PBT45/ | 517 | PLLA | 16 | 175 |
| | Dexa | | | | |

As seen in FIG. 4, results indicated that as the PLLA coated layer thickness increased, the burst release and subsequent release rates were lowered. Release of dexamethasone from coatings containing the least amount of second coated layer exhibited the highest initial burst release. As the weight of the second coated layer of PLLA was increased, the initial burst and subsequent release rate were both reduced significantly. Thus, these experiments show the ability to control the initial burst and sustained release of a relatively small molecular weight bioactive agent from biodegradable coating compositions. Results show the coating weight can be used to adjust the elution rate to meet specific dosage requirements.

FIG. 4 also shows the cumulative percentage of released dexamethasone over time for three different biodegradable coatings. The time t_{1/2} for the coatings containing the least amount of a PLLA second coated layer was on the order of 6 hours. In comparison, as the amount of the PLLA second coated layer was increased, the t_{1/2} was significantly extended. For the intermediate PLLA coating thickness, the t_{1/2} was extended to over 1 day. For the coating composition including PLLA second coating at a weight of 210 μg, approximately 20% of the dexamethasone was released at day 3.

### Example 5

### Elution of Bioactive Agent from Representative Multilayer Coatings Including PLGA Outer Layer

Experiments were conducted to illustrate the effect of multiple coated layers on bioactive agent release profiles.

Stents were provided with a first coated layer containing either PolyActive™ polymer or PLGA with dexamethasone as a model bioactive agent. These coatings were prepared and applied to the stainless steel stents as described previously. For one group of stents, a second coated layer of bioactive-agent free PLGA was applied to adjust bioactive agent release rate. For these coated layers, PLGA was dissolved in tetrahydrofuran to a concentration of 20 milligrams per milliliter, and then applied as a second layer over the existing bioactive agent containing coating by ultrasonic spraying. The stents were then dried in a vacuum oven set at room temperature.

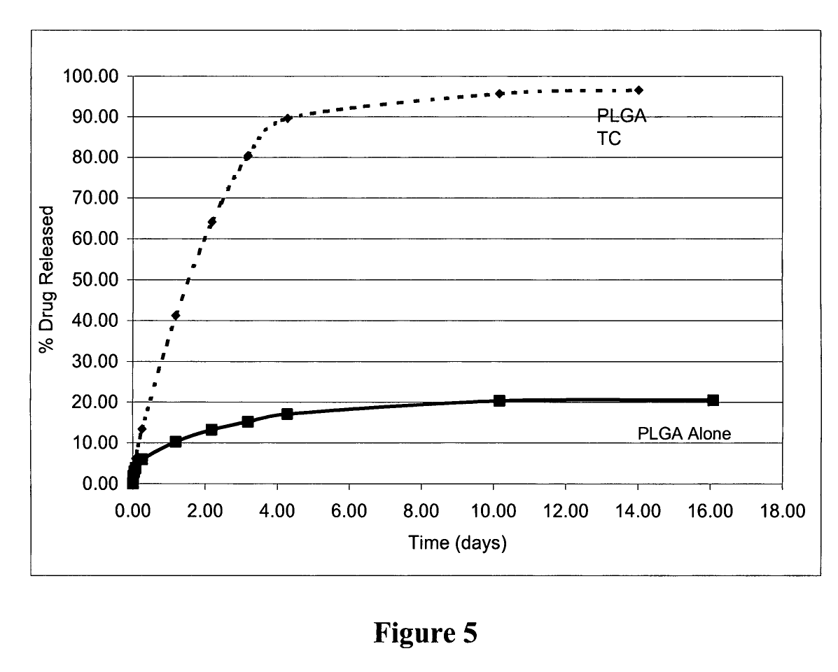

Table 6 lists the coating compositions and bioactive agent weights. FIG. 5 displays the dexamethasone elution results.

TABLE 6
Coating Characteristics

| | | First | | Second | |
|---|---|---|---|---|---|
| | | Layer | Second | Layer | |
| | First Layer | Weight | Layer | Weight | Dexa |
| Coating | Polymer | (μg) | Polymer | (μg) | Weight (μg) |
| PLGA | PLGA/Dexa | 545 | N/A | N/A | 55 |
| PLGA | 300PEGT55PBT45/ | 420 | PLGA | 105 | 42 |
| Topcoat | Dexa | | | | |

As shown in FIG. 5, the release rate of bioactive agent can be controlled by providing a second coated layer composed of PLGA. The stents designated “PLGA Alone” included a single coated layer of PLGA containing dexamethasone, and stents designated “PLGA TC” included a first coated layer of PolyActive™ polymer with dexamethasone, and a second coated layer of PLGA (without bioactive agent). The inclusion of PLGA as a second coated layer over a first coated layer of PolyActive™ polymer provided a controlled release of the bioactive agent.

As shown previously in Example 1, inclusion of a single coated layer of PolyActive™ polymer with bioactive agent provides a very fast release of bioactive agent due to rapid diffusion of dexamethasone through the polymer coating. FIG. 5 illustrates the relatively slow release rate provided by a single coated layer of PLGA containing bioactive agent. When the PLGA is provided as a second coated layer over a first coated layer of PolyActive™ polymer with dexamethasone, the resulting coating provides a controlled release of dexamethasone. An initial release is observed for the first 3-4 days, followed by a sustained release. The initial release shows a controlled release of the dexamethasone that is less than the burst release observed in Example 1 for the PolyActive™ polymer coating containing dexamethasone, yet higher than the release rate for PLGA with dexamethasone.

FIG. 5 also shows the cumulative percentage of released dexamethasone over time for the two different biodegradable coating compositions. For the single layer coating composed of PLGA and dexamethasone, approximately 20% of the bioactive agent was released by Day 16. In contrast, the total amount of dexamethasone released from the multiple layer coating composed of PolyActive™ polymer and PLGA was close to 100% at Day 14. The multilayer coating composed of a first coated layer including PolyActive™ polymer and dexamethasone, and a second coated layer of PLGA (no bioactive agent) exhibited a controlled release profile that included an initial release, followed by a sustained release that approximated zero order kinetics.

Results indicate that inclusion of a second coated layer composed of PLGA can control the initial burst release and subsequent release rate of a relatively small molecular weight bioactive agent from biodegradable coating compositions. Thus, both the initial burst release and subsequent sustained release rate (approximating zero-order release) can be precisely controlled by adjusting the relative coating weights of the first and second coated layers.

### Example 6

### Elution of Bioactive Agent from Representative Multilayer Coatings Including PDLLA Outer Layer

Experiments were conducted to illustrate the effect of multiple coated layers on bioactive agent release profiles.

Stents were provided with a first coated layer containing either PolyActive™ polymer or PDLLA with dexamethasone as a model bioactive agent. These coatings were prepared and applied to the stainless steel stents as described previously. For one group of stents, a second coated layer of bioactive-agent free PDLLA was applied to adjust bioactive agent release rate. For these coated layers, PDLLA was dissolved in tetrahydrofuran to a concentration of 20 milligrams per milliliter, and then applied as a second layer over the existing bioactive agent containing coating by ultrasonic spraying. The stents were then dried in a vacuum oven set at room temperature.

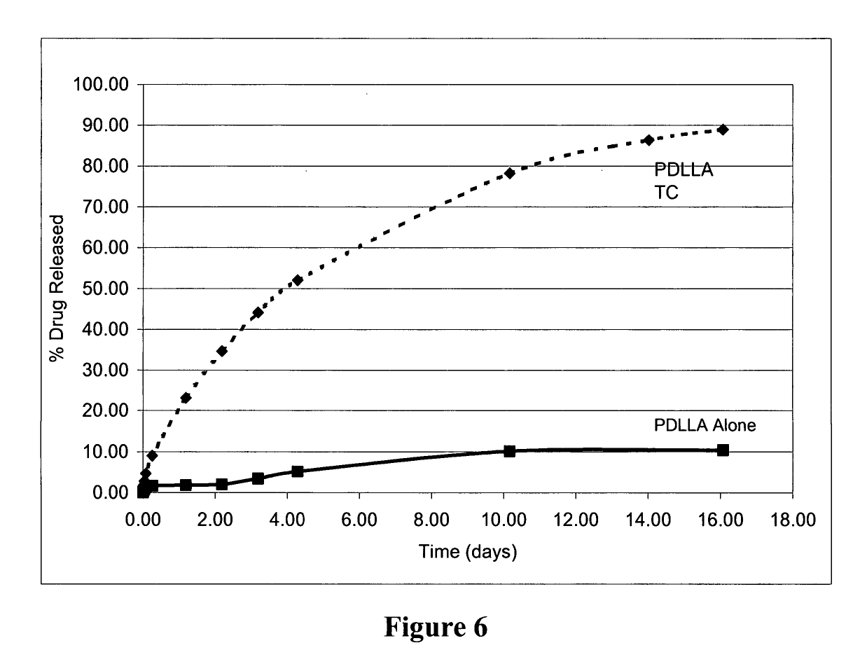

Table 7 lists the coating compositions and bioactive agent weights. FIG. 6 displays the dexamethasone elution results.

TABLE 7
Coating Characteristics

| | | First | | Second | |
|---|---|---|---|---|---|
| | | Layer | Second | Layer | |
| | First Layer | Weight | Layer | Weight | Dexa |
| Coating | Polymer | (μg) | Polymer | (μg) | Weight (μg) |
| PDLLA | PDLLA/Dexa | 535 | N/A | N/A | 54 |
| PDLLA | 300PEGT55PBT45/ | 457 | PDLLA | 86 | 46 |
| Topcoat | Dexa | | | | |

As shown in FIG. 6, the release rate of bioactive agent can be controlled by providing a second coated layer composed of PDLLA. The stents designated “PDLLA Alone” included a single coated layer of PDLLA containing dexamethasone, and stents designated “PDLLA TC” included a first coated layer of PolyActive™ polymer with dexamethasone, and a second coated layer of PDLLA (without bioactive agent). The inclusion of PDLLA as a second coated layer over a first coated layer of PolyActive™ polymer provided a controlled release of the bioactive agent.

As shown previously in Example 1, inclusion of a single coated layer of PolyActive™ polymer with bioactive agent provides a very fast release of bioactive agent due to rapid diffusion of dexamethasone through the polymer coating. FIG. 6 illustrates the relatively slow release rate provided by a single coated layer of PDLLA containing bioactive agent. When the PDLLA is provided as a second coated layer over a first coated layer of PolyActive™ polymer with dexamethasone, the resulting coating provides a controlled release of dexamethasone. An initial release is observed for approximately the first 3 days, followed by a sustained release. The initial release shows a controlled release of the dexamethasone that is less than the burst release observed in Example 1 for the PolyActive™ polymer coating containing dexamethasone, yet higher than the release rate for PDLLA with dexamethasone.

FIG. 6 also shows the cumulative percentage of released dexamethasone over time for the two different biodegradable coating compositions. For the single layer coating composed of PDLLA and dexamethasone, approximately 10% of the bioactive agent was released by Day 16. In contrast, the total amount of dexamethasone released from the multiple layer coating composed of PolyActive™ polymer and PDLLA was approximately 90% at the same time point. The multilayer coating composed of a first coated layer including PolyActive™ polymer and dexamethasone, and a second coated layer of PDLLA (no bioactive agent) exhibited a controlled release profile that included an initial release, followed by a sustained release that approximated zero order kinetics.

Results indicate that inclusion of a second coated layer composed of PDLLA can control the initial burst release and subsequent release rate of a relatively small molecular weight bioactive agent from biodegradable coating compositions. Thus, both the initial burst release and subsequent sustained release rate (approximating zero-order release) can be precisely controlled by adjusting the relative coating weights of the first and second coated layers.

### Example 7

### Elution of Bioactive Agent from Representative Multilayer Coatings Including PolyActive™/Bioactive Agent Base Coat

Stents were provided with a first coated layer containing PolyActive™ polymer with paclitaxel as a model bioactive agent. These coatings were prepared and applied to The stainless steel stents as described previously. A second coated layer of bioactive-agent free P(LLA-CL-GLA) was applied over the base coats to adjust bioactive agent release rate. For one group of stents, a third coated layer of bioactive-agent free PolyActive™ was applied over the second coated layer.

For the base coat, PolyActive™ polymer was dissolved into chloroform with paclitaxel, as described previously. The concentration of PolyActive™ polymer was 40 milligram per milliliter. For coated layers that did not contain bioactive agent, the P(LLA-CL-GLA) or PolyActive™ polymer was dissolved into chloroform, as described previously, to a concentration of 20 milligram per milliliter.

Coating compositions were applied by ultrasonic spraying. The coated stents were then dried in a vacuum oven set at room temperature.

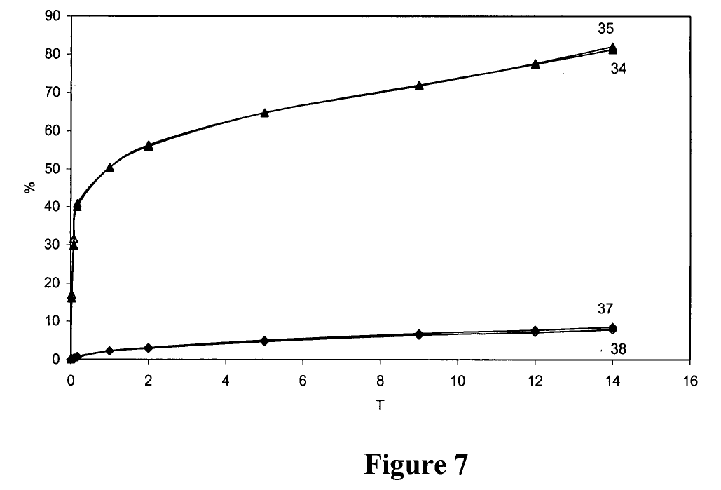

Table 8 lists the coating compositions and bioactive agent weights. FIG. 7 displays the paclitaxel elution results.

TABLE 8
Coating Characteristics

| | | | Second | |
|---|---|---|---|---|
| | Base Coat Layer | Wt % in | Coat Layer | Third Coat Layer |
| Coating | Composition | Base Coat | Composition | Composition |
| 34-36, | PTX/1000PEGT55PBT45 | 33/67 | P(LLA-CL- | 1000PEGT80PBT20 |
| 42 | | | GLA) | |
| 37-39, | PTX/1000PEGT55PBT45 | 33/67 | P(LLA-CL- | N/A |
| 43 | | | GLA) | |

For each group of stents, two samples were subjected to elution studies, one sample was subjected to surface characterization (optical, Raman and SEM), and one sample was subjected to mechanical studies.

Elution Studies

As shown in FIG. 7, the release rate of bioactive agent can be controlled by providing a second coated layer composed of P(LLA-CL-GLA). The inclusion of P(LLA-CL-GLA) as a second coated layer over a first coated layer of PolyActive™ polymer provided a controlled release of the bioactive agent. The addition of a third coated layer composed of PolyActive™ did not further decrease the elution rate of paclitaxel from the coatings. For samples 34 and 35, approximately 50% of paclitaxel was released within the first 24 hours. At 14 days, over 81% of the paclitaxel had been released. In contrast, samples 37 and 38 (including two coated layers only) released approximately 2% of paclitaxel within the first 24 hours, and at 14 days, 8.5% or less of the paclitaxel had been released from these samples. Thus, the two-layer sample not only had a lower initial release phase, but the initial phase was followed by a slower release rate over time (approximating zero-order release kinetics). Thus, more paclitaxel remained in the 2-layer coatings at the conclusion of the assay, which in turn can provide a longer therapeutic treatment period.

Results indicate that inclusion of a second coated layer composed of P(LLA-CL-GLA) can control the initial burst release and subsequent release rate of a relatively small molecular weight bioactive agent from biodegradable coating compositions. Thus, both the initial burst release and subsequent sustained release rate (approximating zero-order release) can be precisely controlled by adjusting the relative coating weights of the first and second coated layers. Results also indicate that this slower release rate can be accelerated to an intermediate rate by inclusion of a third coated layer (an outer layer, or top coat) over the two coated layers, wherein the third coating composition is composed of PolyActive™. This latter feature can be utilized to fine-tune release profiles for selected bioactive agents, depending upon the final application of the device.

Surface Characterization

Surface analysis of the samples was performed to characterize the polymer coating on the stents, observing coating quality, uniformity, and mixing of the components.

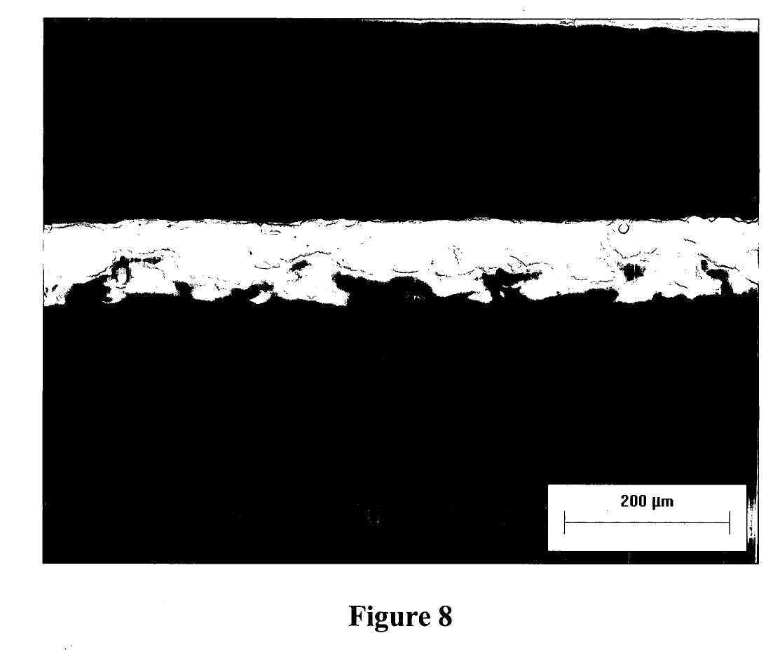

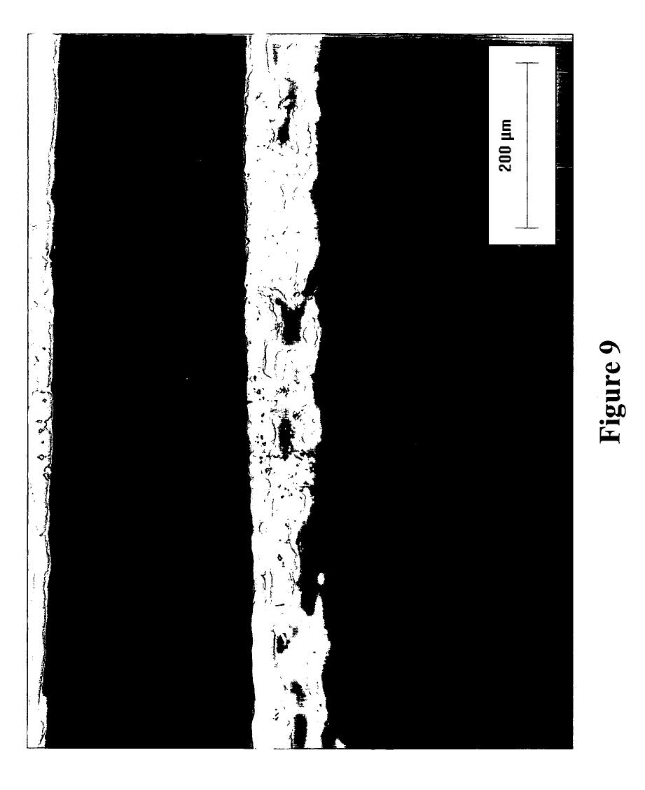

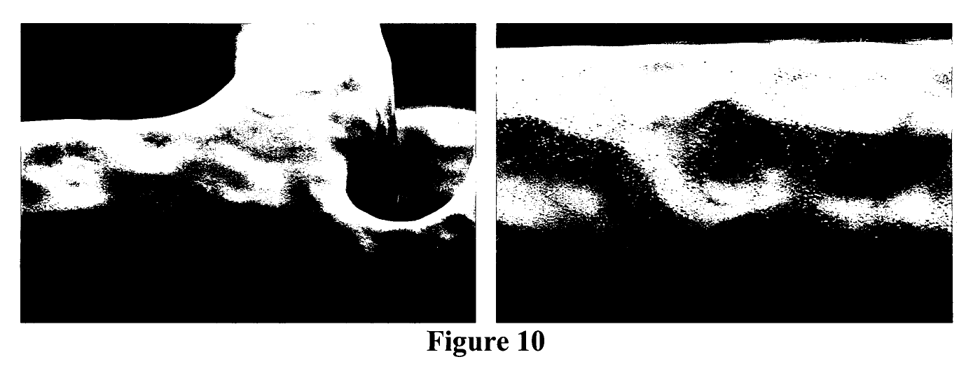

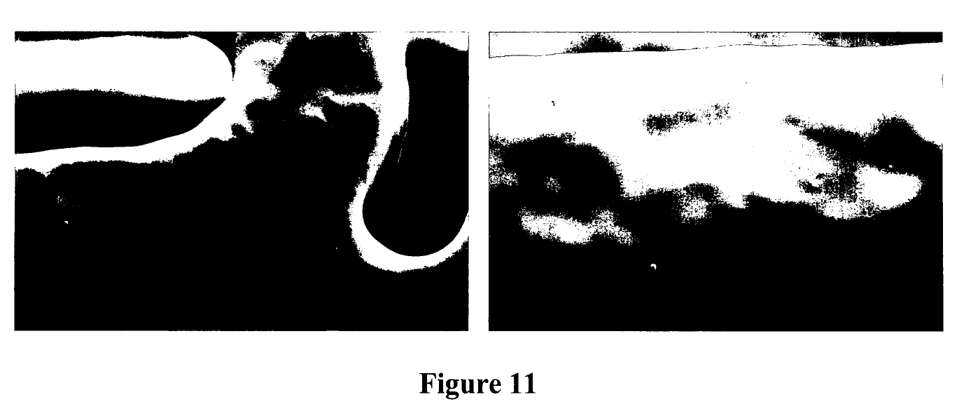

Optical and SEM images showed coatings on the metal stents had no webbing, cracking or coating delamination. No crystals of the paclitaxel were seen in the optical or SEM images. Overall, coatings appeared uniform across each stent. FIGS. 8 and 9 show Optical images for Coatings 36 and 39, respectively (100× magnification). FIGS. 10 and 11 show SEM images of the Coatings 36 and 39. SEM images showed that the coatings including a PolyActive™ topcoat possessed a bumpy pattern on the surface; however, there was no bumpy pattern on the surface of the stent including an top coat of P(LLA-CL-GLA). Thus, it appears the presence of PolyActive™ polymer at the surface (outer coated layer) can present a bumpy surface feature on the device.

Confocal Raman images showed the distribution of the coating components on each stent. Cross-sectional Raman images (taken perpendicular to the metal stent struts) were obtained over regions 50 μm in width and 10 or 15 μm in depth. In a cross-sectional image, the air above the coating had no Raman signal, the coating had a strong Raman signal, and the metal below the coating had no Raman signal.

At each pixel in the image, an entire Raman spectrum was obtained. An augmented classical least squares (CLS) algorithm was applied to deconvolute the data set into images of the individual components using reference spectra for P(LLA-CL-GLA), 1000PEGT55PBT45, 1000PETT80PBT20, paclitaxel, and Parylene™.

In all stents examined, the biodegradable polymers and the paclitaxel appeared to mix completely with no large segregations or drug crystals formed within the coatings. The images showed that the paclitaxel mixed into the biodegradable polymers uniformly, with no large phase segregation or crystals formed in the coatings. Additionally, the midcoat and topcoat layers of the coating were clearly visible in the Raman images and did not appear to have mixed with each other or with the paclitaxel. Thus, coating layer integrity appears to have been retained in these samples.

Mechanical Testing

After conventional balloon expansion of a selected stent, visual inspection of the stent coating under 6.3× magnification was conducted to determine coating quality on the stent. Inspection revealed that stent coatings with multiple coated layers composed of paclitaxel, PolyActive™ and P(LLA-CL-GLA) as a second polymer provided acceptable coatings. Acceptable stent coatings were characterized in appearance, for example, by minimal surface cracking, minimal webbing between stent struts, smooth texture to the coating surface, and coating adherence to the stent substrate.

### Example 8

### Elution of Bioactive Agent from Representative Multilayer Coatings Including PolyActive™/Bioactive Agent Base Coat

Stents were provided with a first coated layer containing PolyActive™ polymer with paclitaxel as a model bioactive agent. These coatings were prepared and applied to The stainless steel stents as described previously. A second coated layer of bioactive-agent free P(LLA-EG-EOP) was applied over the base coats to adjust bioactive agent release rate. For one group of stents, a third coated layer of bioactive-agent free PolyActive™ was applied over the second coated layer.

For the base coat, PolyActive™ polymer was dissolved into chloroform with paclitaxel, as described previously. The concentration of PolyActive™ polymer was 40 milligram per milliliter. For coated layers that did not contain bioactive agent, the P(LLA-CL-GLA) or PolyActive™ polymer was dissolved into chloroform, as described previously, to a concentration of 20 milligram per milliliter.

Coating compositions were applied by ultrasonic spraying. The coated stents were then dried in a vacuum oven set at room temperature.

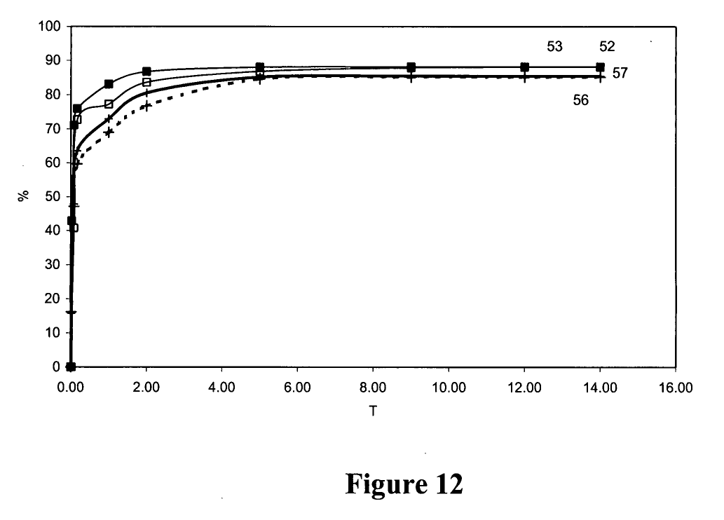

Table 9 lists the coating compositions and bioactive agent weights. FIG. 12 displays the paclitaxel elution results.

TABLE 9
Coating Characteristics

| | | | Second | |
|---|---|---|---|---|
| | Base Coat Layer | Wt % in | Coat Layer | Third Coat Layer |
| Coating | Composition | Base Coat | Composition | Composition |
| 52-55 | PTX/1000PEGT55PBT45 | 33/67 | P(LLA-EG- | 1000PEGT80PBT20 |
| | | | EOP) | |
| 56-59 | PTX/1000PEGT55PBT45 | 33/67 | P(LLA-EG- | N/A |
| | | | EOP) | |

For each group of stents, two samples were subjected to elution studies, one sample was subjected to surface characterization (optical, Raman and SEM), and one sample was subjected to mechanical studies.

Elution Studies

As shown in FIG. 12, the release rate of bioactive agent can be controlled by providing a second coated layer composed of P(LLA-EG-EOP). The inclusion of a PolyActive™ topcoat increased the initial release phase (initial burst). However, after the initial burst, elution rates appeared similar. For samples 52 and 53 (included PolyActive™ topcoat), approximately 83-84% of paclitaxel was released within the first 24 hours. For samples 56 and 57 (including two coated layers only, topcoat of P(LLA-EG-EOP)) released approximately 69% and 73% of paclitaxel within the first 24 hours. At 14 days, the PolyActive™ topcoat samples had released approximately 88% of paclitaxel, while the P(LLA-EG-EOP) topcoat samples had released approximately 85% of paclitaxel.

When comparing FIG. 12 with FIG. 7, the dramatic effect can be noted from the P(LLA-CL-GLA) polymer in the coated composition. Inclusion of the P(LLA-CL-GLA) as a topcoat provided significant reduction in initial release of bioactive agent, as well as sustained release of bioactive agent. Topcoats composed of either PolyActive™ or P(LLA-EG-EOP) possessed significantly higher initial release, as well as total release of bioactive agent over the time course of the study, as P(LLA-EG-EOP) is more hydrophilic, and thus, more similar to PolyActive™.

Surface Characterization

Surface analysis of the samples was performed to characterize the polymer coating on the stents, observing coating quality, uniformity, and mixing of the components.

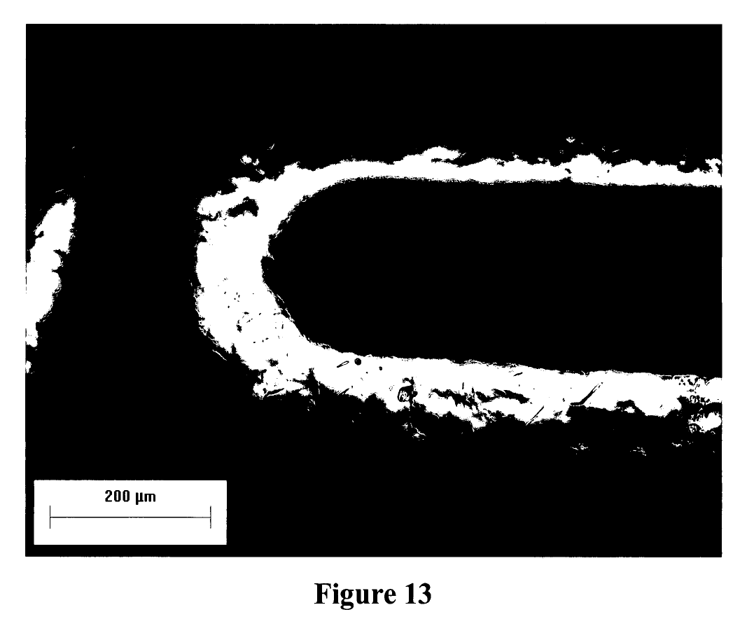

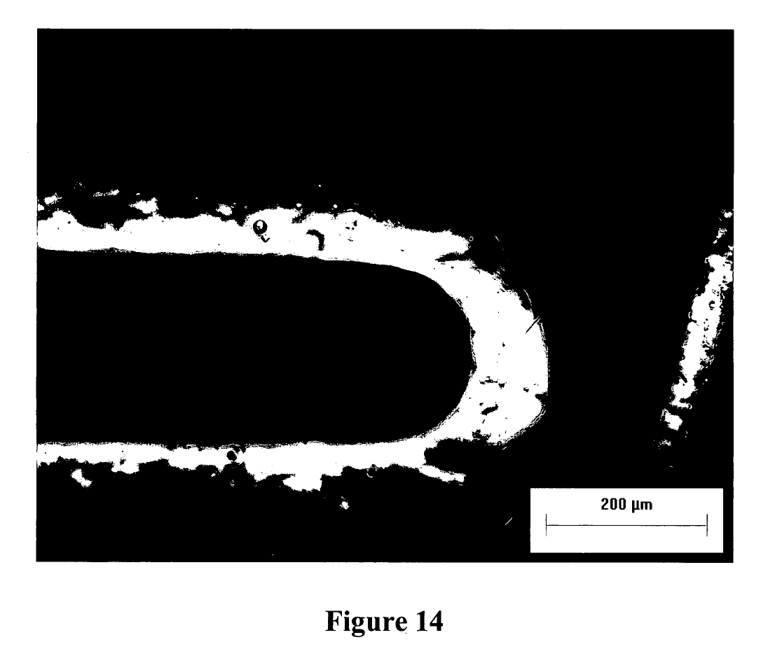

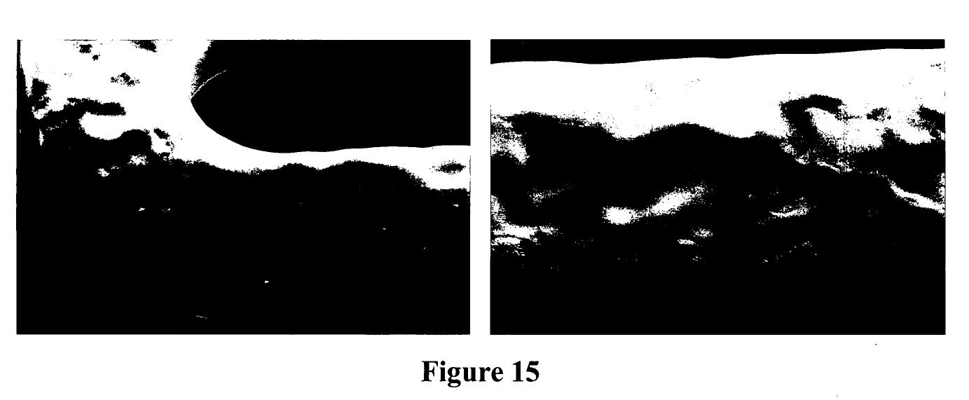

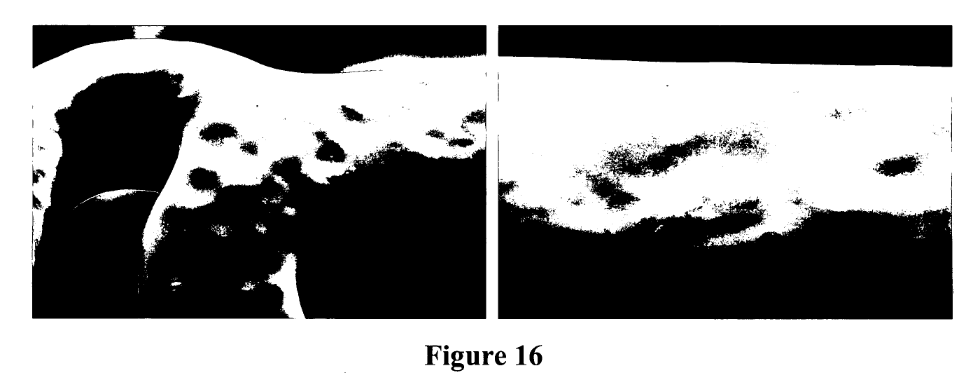

Optical and SEM images showed coatings on the metal stents had no webbing, cracking or coating delamination. No crystals of the paclitaxel were seen in the optical or SEM images. Overall, coatings appeared uniform across each stent. Some waviness in the coatings was observed. FIGS. 13 and 14 show Optical images for Coatings 54 and 58, respectively (100× magnification). FIGS. 15 and 16 show SEM images of the Coatings 54 and 58. SEM images showed that the coatings including a PolyActive™ topcoat possessed a bumpy pattern on the surface; however, there was no bumpy pattern on the surface of the stent including an top coat of P(LLA-EG-EOP). Thus, it appears the presence of PolyActive™ polymer at the surface (outer coated layer) can present a bumpy surface feature on the device.

Confocal Raman images showed the distribution of the coating components on each stent. Cross-sectional Raman images (taken perpendicular to the metal stent struts) were obtained over regions 50 μm in width and 10 or 15 μm in depth. In a cross-sectional image, the air above the coating had no Raman signal, the coating had a strong Raman signal, and the metal below the coating had no Raman signal.

At each pixel in the image, an entire Raman spectrum was obtained. An augmented classical least squares (CLS) algorithm was applied to deconvolute the data set into images of the individual components using reference spectra for P(LLA-EG-EOP), 1000PEGT55PBT45, 1000PETT80PBT20, paclitaxel, and Parylene™.

In all stents examined, the biodegradable polymers and the paclitaxel appeared to mix completely with no large segregations or drug crystals formed within the coatings. The images showed that the paclitaxel mixed into the biodegradable polymers uniformly, with no large phase segregation or crystals formed in the coatings. Additionally, the midcoat and topcoat layers of the coating were clearly visible in the Raman images and did not appear to have mixed with each other or with the paclitaxel. Thus, coating layer integrity appears to have been retained in these samples.

Mechanical Testing

After conventional balloon expansion of a selected stent, visual inspection of the stent coating under 6.3× magnification was conducted to determine coating quality on the stent. Inspection revealed that stent coatings with multiple coated layers composed of paclitaxel, PolyActive™ and P(LLA-EG-EOP) as a second polymer provided acceptable coatings. Acceptable stent coatings were characterized in appearance, for example, by minimal surface cracking, minimal webbing between stent struts, smooth texture to the coating surface, and coating adherence to the stent substrate.

### Example 9

### Elution of Bioactive Agent from Representative Multilayer Coatings Including P(LLA-CL-GLA)/Bioactive Agent Base Coat

Stents were provided with a first coated layer containing P(LLA-CL-GLA) copolymer with paclitaxel as a model bioactive agent. These coatings were prepared and applied to The stainless steel stents as described previously. For the base coat, P(LLA-CL-GLA) copolymer was dissolved into chloroform with paclitaxel, as described previously. The concentration of P(LLA-CL-GLA) copolymer was 40 milligram per milliliter.

Two groups of stents were prepared. In the first group, a second coated layer containing PolyActive™ was applied. In a second group, a total of three coated layers were applied to the stents; that is, a second coated layer composed of P(LLA-CL-GLA) was applied over the base coat, and a third coated layer composed of PolyActive™ was applied over the second coated layer. Thus, for both groups, the top coat was composed of PolyActive™, and the presence of a mid-coated layer of P(LLA-CL-GLA) was included in some of the samples.

For coated layers that did not contain bioactive agent, the P(LLA-CL-GLA) or PolyActive™ polymer was dissolved into chloroform, as described previously, to a concentration of 20 milligram per milliliter.

Coating compositions were applied by ultrasonic spraying. The coated stents were then dried in a vacuum oven set at room temperature.

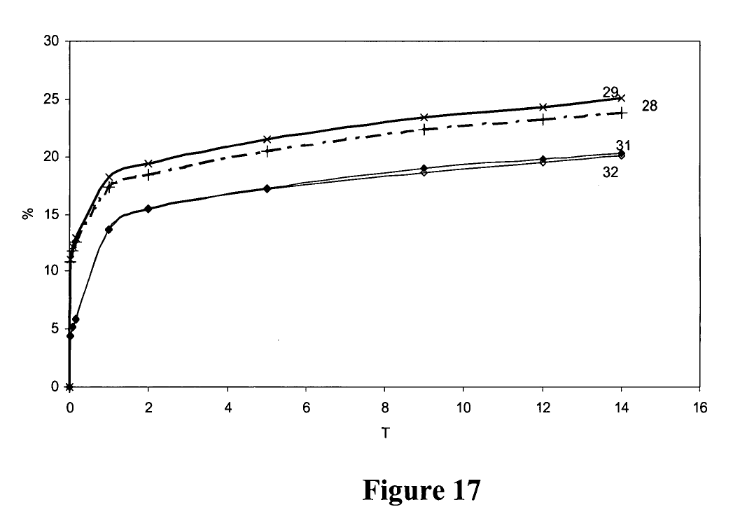

Table 10 lists the coating compositions and bioactive agent weights. FIG. 17 displays the paclitaxel elution results.

TABLE 10
Coating Characteristics

| | Base Coat Layer | Wt % in | Second Coat Layer | Third Coat Layer |
|---|---|---|---|---|
| Coating | Composition | Base Coat | Composition | Composition |
| 28-30, | PTX/P(LLA-CL-GLA) | 33/67 | 1000PEGT80PBT20 | N/A |
| 40 | | | | |
| 31-33, | PTX/P(LLA-CL-GLA) | 33/67 | P(LLA-CL-GLA) | 1000PEGT80PBT20 |
| 41 | | | | |

For each group of stents, two samples were subjected to elution studies, one sample was subjected to surface characterization (optical, Raman and SEM), and one sample was subjected to mechanical studies.

Elution Studies

As shown in FIG. 17, utilization of P(LLA-CL-GLA) as an intermediate coated layer (second coated layer between the base coat and top coat) decreased the rate of paclitaxel elution. The inclusion of a PolyActive™ topcoat increased the initial release phase (initial burst). However, after the initial burst, elution rates appeared to be similar. For samples 31 and 32 (included second coated layer of P(LLA-CL-GLA)), approximately 14% of paclitaxel was released within the first 24 hours. For samples 28 and 29 (no intermediate coated layer) released approximately 17% and 18% of paclitaxel within the first 24 hours. At 14 days, the 3-layer samples had released approximately 20% of paclitaxel, while the 2-layer samples had released approximately 24-25% of paclitaxel.

Surface Characterization

Surface analysis of the samples was performed to characterize the polymer coating on the stents, observing coating quality, uniformity, and mixing of the components.

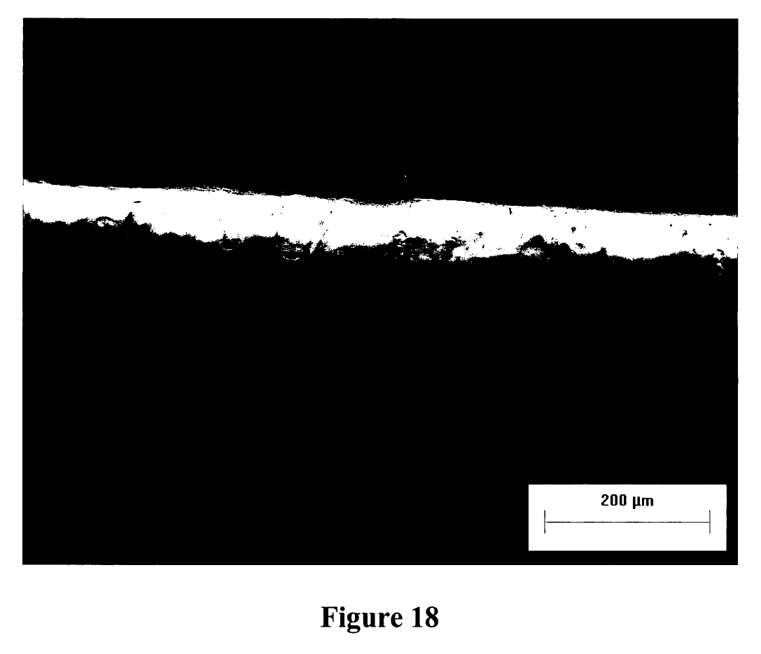

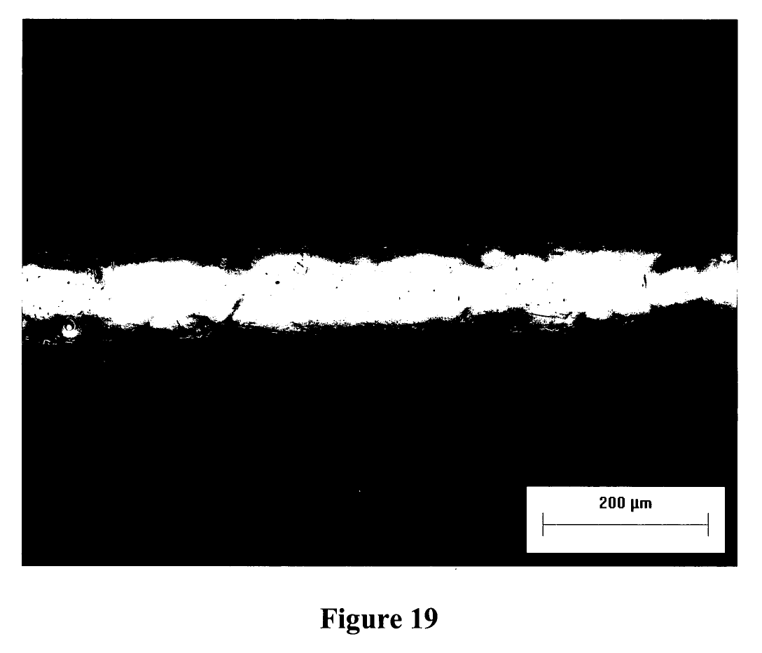

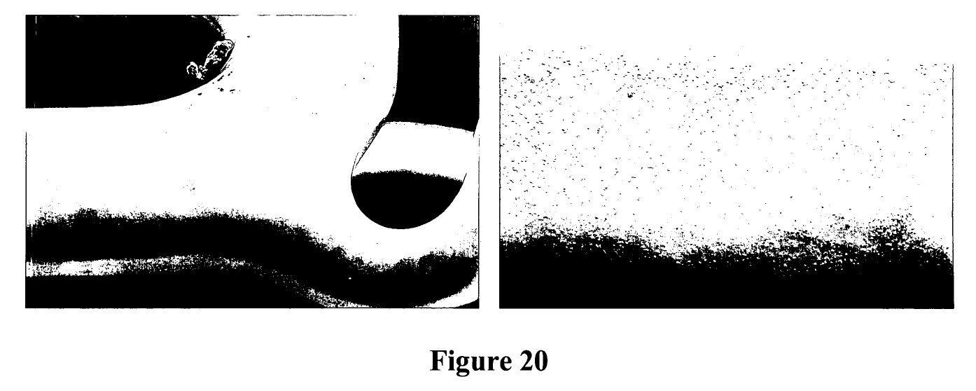

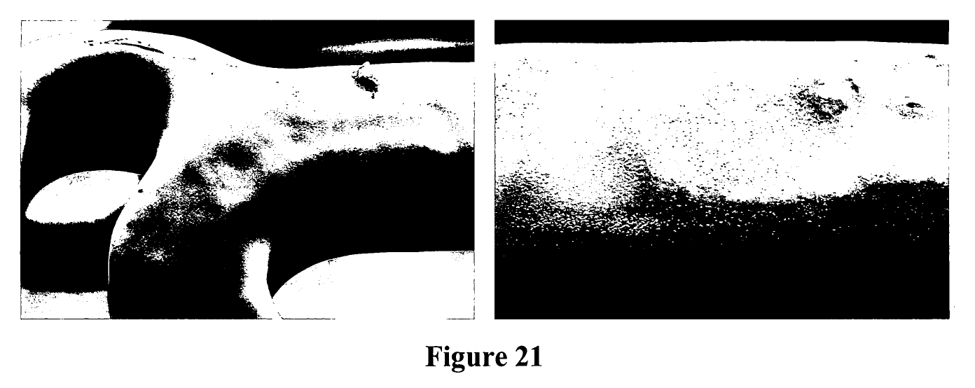

Optical and SEM images showed coatings on the metal stents had no webbing, cracking or coating delamination. No crystals of the paclitaxel were seen in the optical or SEM images. Overall, coatings appeared uniform across each stent. FIGS. 18 and 19 show optical images for Coatings 30 and 33, respectively (100× magnification). FIGS. 20 and 21 show SEM images of the Coatings 30 and 33. SEM images revealed a bumpy patterned surface on the surface of the coated stents.

Confocal Raman images showed the distribution of the coating components on each stent. Cross-sectional Raman images (taken perpendicular to the metal stent struts) were obtained over regions 50 μm in width and 10 or 15 μm in depth. In a cross-sectional image, the air above the coating had no Raman signal, the coating had a strong Raman signal, and the metal below the coating had no Raman signal.

At each pixel in the image, an entire Raman spectrum was obtained. An augmented classical least squares (CLS) algorithm was applied to deconvolute the data set into images of the individual components using reference spectra for P(LLA-CL-GLA), 1000PEGT55PBT45, 1000PETT80PBT20, paclitaxel, and Parylene™.

In all stents examined, the biodegradable polymers and the paclitaxel appeared to mix completely with no large segregations or drug crystals formed within the coatings. The images showed that the paclitaxel mixed into the biodegradable polymers uniformly, with no large phase segregation or crystals formed in the coatings. Additionally, the midcoat and topcoat layers of the coating were clearly visible in the Raman images and did not appear to have mixed with each other or with the paclitaxel. Thus, coating layer integrity appears to have been retained in these samples.

Mechanical Testing

After conventional balloon expansion of a selected stent, visual inspection of the stent coating under 6.3× magnification was conducted to determine coating quality on the stent. Inspection revealed that stent coatings with multiple coated layers composed of paclitaxel, PolyActive™ and P(LLA-CL-GLA) as a second polymer provided acceptable coatings. Acceptable stent coatings were characterized in appearance, for example, by minimal surface cracking, minimal webbing between stent struts, smooth texture to the coating surface, and coating adherence to the stent substrate.

### Example 10

### Elution of Bioactive Agent from Representative Multilayer Coatings Including P(LLA-EG-EOP)/Bioactive Agent Base Coat

Stents were provided with a first coated layer containing P(LLA-EG-EOP) copolymer with paclitaxel as a model bioactive agent. These coatings were prepared and applied to The stainless steel stents as described previously. For the base coat, P(LLA-EG-EOP) copolymer was dissolved into chloroform with paclitaxel, as described previously. The concentration of P(LLA-EG-EOP) copolymer was 40 milligram per milliliter.

Two groups of stents were prepared. In the first group, a second coated layer containing PolyActive™ was applied. In a second group, a total of three coated layers were applied to the stents; that is, a second coated layer composed of P(LLA-EG-EOP) was applied over the base coat, and a third coated layer composed of PolyActive™ was applied over the second coated layer. Thus, for both groups, the top coat was composed of PolyActive™, and the presence of a mid-coated layer of P(LLA-EG-EOP) was included in some of the samples.

For coated layers that did not contain bioactive agent, the P(LLA-EG-EOP) or PolyActive™ polymer was dissolved into chloroform, as described previously, to a concentration of 20 milligram per milliliter.

Coating compositions were applied by ultrasonic spraying. The coated stents were then dried in a vacuum oven set at room temperature.

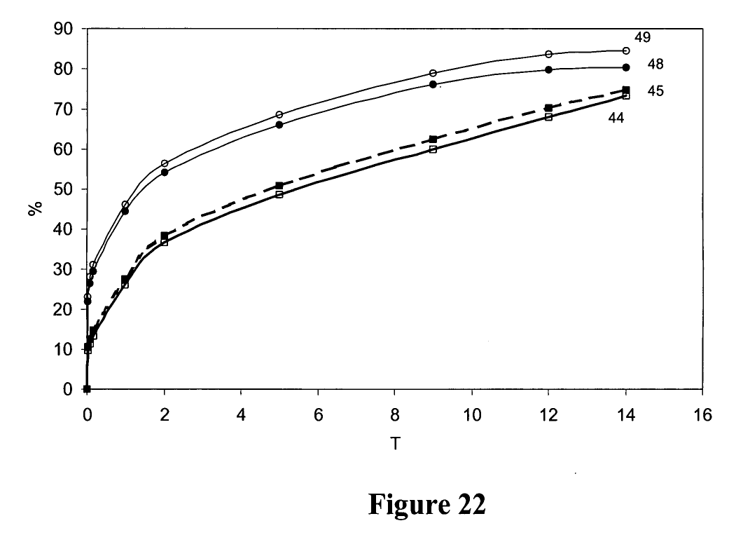

Table 11 lists the coating compositions and bioactive agent weights. FIG. 22 displays the paclitaxel elution results.

TABLE 11
Coating Characteristics

| | Base Coat Layer | Wt % in | Second Coat Layer | Third Coat Layer |
|---|---|---|---|---|
| Coating | Composition | Base Coat | Composition | Composition |
| 44-47 | PTX/P(LLA-EG-EOP) | 33/67 | P(LLA-EG-EOP) | 1000PEGT80PBT20 |
| 48-51 | PTX/P(LLA-EG-EOP) | 33/67 | 1000PEGT80PBT20 | N/A |

For each group of stents, two samples were subjected to elution studies, one sample was subjected to surface characterization (optical, Raman and SEM), and one sample was subjected to mechanical studies.

Elution Studies

As shown in FIG. 22, utilization of P(LLA-EG-EOP) as an intermediate coated layer (second coated layer between the base coat and top coat) decreased the rate of paclitaxel elution. The inclusion of a PolyActive™ topcoat increased the initial release phase (initial burst). The differences in initial release phase is thought to be a result of the PolyActive™ topcoat.

However, after the initial burst, elution rates appeared similar. For samples 44 and 45 (included second coated layer of P(LLA-EG-EOP)), approximately 26%-28% of paclitaxel was released within the first 24 hours. For samples 48 and 49 (no intermediate coated layer) released approximately 44%-46% of paclitaxel within the first 24 hours. At 14 days, the 3-layer samples had released approximately 73%-75% of paclitaxel, while the 2-layer samples had released approximately 80%-84% of paclitaxel.

Results of Examples 9 and 10 suggest that use of P(LLA-CL-GLA) copolymer appears to slow the release of a small molecule (paclitaxel) more than the P(LLA-EG-EOP) copolymer.

Surface Characterization

Surface analysis of the samples was performed to characterize the polymer coating on the stents, observing coating quality, uniformity, and mixing of the components.

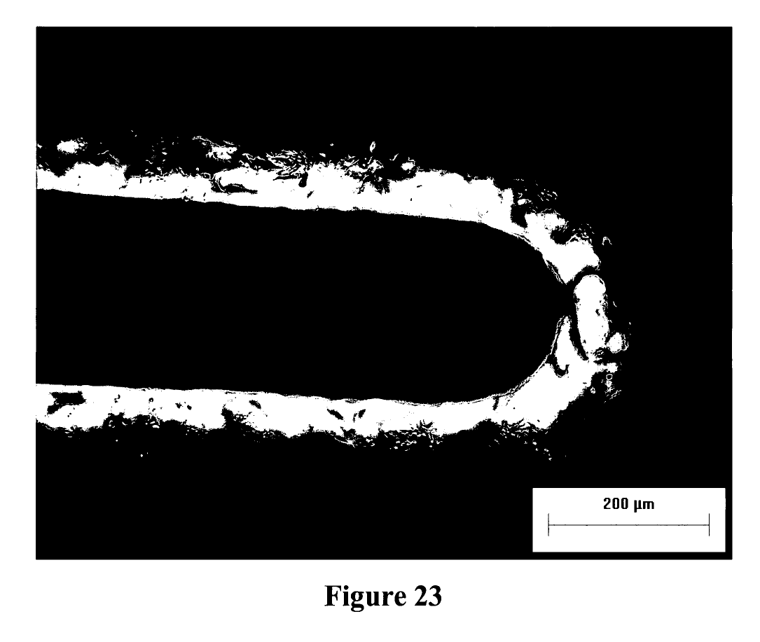

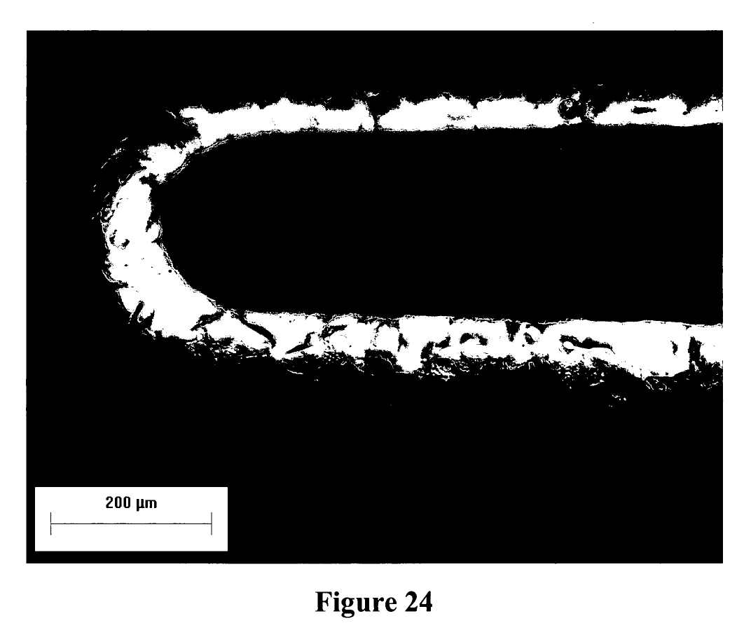

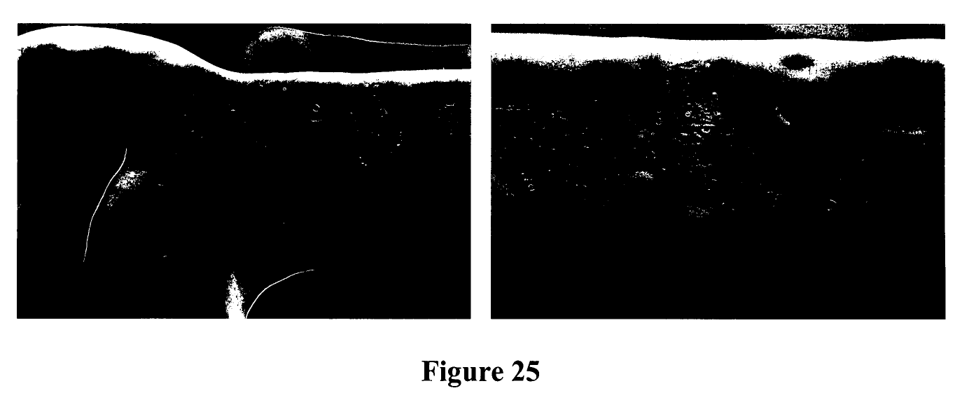

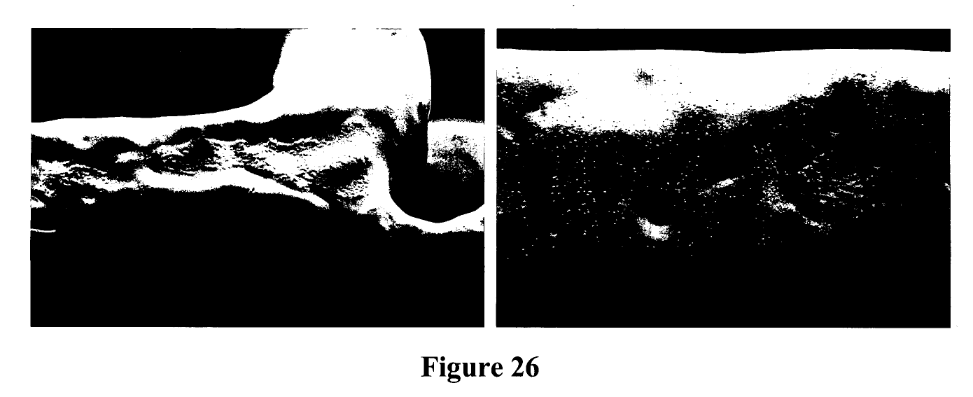

Optical and SEM images showed coatings on the metal stents had no webbing, cracking or coating delamination. No crystals of the paclitaxel were seen in the optical or SEM images. Overall, coatings appeared uniform across each stent. FIGS. 23 and 24 show optical images for Coatings 50 and 46, respectively (100× magnification). FIGS. 25 and 26 show SEM images of the Coatings 50 and 46. SEM images revealed a bumpy patterned surface on the surface of the coated stents.

Confocal Raman images showed the distribution of the coating components on each stent. Cross-sectional Raman images (taken perpendicular to the metal stent struts) were obtained over regions 50 μm in width and 10 or 15 μm in depth. In a cross-sectional image, the air above the coating had no Raman signal, the coating had a strong Raman signal, and the metal below the coating had no Raman signal.

At each pixel in the image, an entire Raman spectrum was obtained. An augmented classical least squares (CLS) algorithm was applied to deconvolute the data set into images of the individual components using reference spectra for P(LLA-CL-GLA), 1000PEGT55PBT45, 1100PETT80PBT20, paclitaxel, and Parylene™.

In all stents examined, the biodegradable polymers and the paclitaxel appeared to mix completely with no large segregations or drug crystals formed within the coatings. The images showed that the paclitaxel mixed into the biodegradable polymers uniformly, with no large phase segregation or crystals formed in the coatings. Additionally, the midcoat and topcoat layers of the coating were clearly visible in the Raman images and did not appear to have mixed with each other or with the paclitaxel. Thus, coating layer integrity appears to have been retained in these samples.

Mechanical Testing

After conventional balloon expansion of a selected stent, visual inspection of the stent coating under 6.3× magnification was conducted to determine coating quality on the stent. Inspection revealed that stent coatings with multiple coated layers composed of paclitaxel, PolyActive™ and P(LLA-EG-EOP) as a second polymer provided acceptable coatings. Acceptable stent coatings were characterized in appearance, for example, by minimal surface cracking, minimal webbing between stent struts, smooth texture to the coating surface, and coating adherence to the stent substrate.

In designing a coating that can provide controlled release of a bioactive agent, it is desirable to have the capability to modulate the shape of the release curve. The time profile of the release of the bioactive agent can range from immediate release where the drug elutes all at once (much like a step function) to an extremely slow, linear (zero order) release, where the drug is evenly released over many months or years. Depending upon the drug and the condition being treated, there are a variety of release profiles that are of interest. The objective of creating coatings including multiple coated layers of polymers is to be able to attain the broad range of release profiles that lie between a step function and a low-slope, zero-order release.

One of the primary strategies to control the release of a bioactive agent, is to limit the initial release (or “burst”) of bioactive agent. If this can be achieved, then more bioactive agent is available at later times for a more extended release duration. The inclusion of multiple coated layers within a coating described herein is designed to limit or even eliminate the burst of bioactive agent from the coating. The bioactive agent still remaining in the coating after the initial burst is then released to the site of action over a longer time period. The shape of the release profile (percentage of drug released versus time) after the burst can be controlled to be linear or logarithmic or some more complex shape, again depending on the composition of the coated layers of polymers and bioactive agent in the coating.

Once a therapeutic range has been determined (for example, by a physician), the inventive coatings can be adjusted to provide the bioactive agent at a dosage that is within the therapeutic range. The inventive compositions provide improved means to control release of the bioactive agent, thus providing enhanced ability to deliver bioactive agent at desired rates and amounts.

The results discussed in the preceding Examples show that the inventive multiple layer coatings can limit initial release of bioactive agent and provide control over the shape of the release profile curves.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims. All patents, patent documents, and publications cited herein are hereby incorporated by reference as if individually incorporated.

We claim:

1. An implantable medical article having a bioactive agent releasing coating at a surface, the coating comprising:
(a) a first coated layer comprising bioactive agent and a first biodegradable polymer, wherein the first biodegradable polymer is a copolymer of polyalkylene glycol terephthalate and an aromatic polyester; and
(b) a second coated layer covering at least a portion of the first coated layer and comprising a second biodegradable polymer,
wherein the first biodegradable polymer and the second biodegradable polymer are different,
and wherein the second biodegradable polymer is selected to have a slower bioactive agent release rate relative to the first biodegradable polymer.

2. The article according to claim 1 wherein the polyalkylene glycol terephthalate is selected from the group of polyethylene glycol terephthalate, polypropylene glycol terephthalate, polybutylene glycol terephthalate, and combinations of these.

3. The article according to claim 2 wherein the polyalkylene glycol is polyethylene glycol.

4. The article according to claim 1 wherein the polyester is selected from polyethylene terephthalate, polypropylene terephthalate, polybutylene terephthalate, and combinations of these.

5. The article according to claim 4 wherein the polyester is polybutylene terephthalate.

6. The article according to claim 1 wherein the second biodegradable polymer is more hydrophobic relative to the first biodegradable polymer.

7. The article according to claim 1 wherein the second biodegradable polymer comprises a polymer derived from monomers selected from lactic acid, glycolic acid, caprolactone, ethylene glycol, and ethyloxyphosphate.

8. The article according to claim 1 wherein the bioactive agent is a hydrophobic small molecule bioactive agent.

9. The article according to claim 8 wherein the bioactive agent has a molecular weight of 1500 or less.

10. The article according to claim 9 wherein the bioactive agent is selected from anti-proliferative agents, anti-inflammatory agents, immunosuppressive agents, small molecule antibiotics, estrogens, and combinations of any of these.

11. The article according to claim 10 wherein the bioactive agent is selected from actinomycin D, paclitaxel, taxane, dexamethasone, prednisolone, tranilast, cyclosporine, everolimus, mycophenolic acid, sirolimus, tacrolimus, estradiol, and combinations of any of these.

12. The article according to claim 1 wherein total bioactive agent content within the first coated layer is 50% or less.

13. The article according to claim 1 wherein two or more bioactive agents are included in the coating.

14. The article according to claim 9 wherein upon placement of the article in a biological environment, the bioactive agent is released, and wherein release is 10% or less within 24 hours after placement of the article in the biological environment.

15. The article according to claim 14 wherein the bioactive agent release is 2% or less within 24 hours after placement of the article in the biological environment.

16. The article according to claim 14 wherein the bioactive agent release is 20% or less within seven days after placement of the article in the biological environment.

17. The article according to claim 9 wherein the bioactive agent is released at a therapeutically effective concentration for at least one week, when the article is implanted in a patient.

18. The article according to claim 9 wherein the bioactive agent is released at a therapeutically effective concentration for at least four weeks, when the article is implanted in a patient.

19. The article according to claim 1 wherein the coating is provided on a surface of the article that comprises less than 100% of total article surface area.

20. The article according to claim 1 wherein the bioactive agent releasing coating further comprises a coating layer comprising parylene, silane, siloxane, polyurethane, polybutadiene, polycarbodiimide, or a combination of any of these.

21. The article according to claim 1 wherein the surface is proivded with a surface texture.

22. The article according to claim 1 wherein the article is a stent, graft, catheter, valve, cardiac device, ophthalmic device, or wound dressing.

23. An implantable medical article having a bioactive agent releasing coating at a surface, the coating comprising:
(a) a first coated layer comprising bioactive agent and a second biodegradable polymer; and
(b) an outer coated layer comprising a polyetherester copolymer that is a copolymer of polyalkylene glycol terephthalate and an aromatic polyester,
wherein the polyetherester copolymer and the second biodegradable polymer are different,
and wherein the second biodegradable polymer is selected to have a slower bioactive agent release rate relative to the polyetherester copolymer.

24. The article according to claim 23 wherein the coating further comprises one or more intermediate coated layers positioned between the first coated layer and the outer coated layer.

25. The article according to claim 23 wherein one or more of the intermediate coated layers comprises a polymer other than the polyetherester copolymer.

26. The article according to claim 23 wherein the polyalkylene glycol terephthalate is selected from the group of polyethylene glycol terephthalate, polypropylene glycol terephthalate, polybutylene glycol terephthalate, and combinations of these.

27. The article according to claim 23 wherein the polyester is selected from polyethylene terephthalate, polypropylene terephthalate, polybutylene terephthalate, and combinations of these.

28. The article according to claim 27 wherein the polyalkylene glycol terephthalate is polyethylene glycol terephthalate and the polyester is polybutylene terephthalate.

29. The article according to claim 23 wherein the second biodegradable polymer is more hydrophobic relative to the polyetherester copolymer.

30. The article according to claim 24 wherein the second biodegradable polymer comprises a polymer derived from monomers selected from lactic acid, glycolic acid, caprolactone, ethylene glycol, and ethyloxyphosphate.

31. The article according to claim 23 wherein the bioactive agent has a molecular weight of 1500 or less.

32. The article according to claim 31 wherein the bioactive agent is selected from anti-proliferative agents, anti-inflammatory agents, immunosuppressive agents, small molecule antibiotics, estrogens, and combinations of any of these.

33. The article according to claim 32 wherein the bioactive agent is selected from actinomycin D, paclitaxel, taxane, dexamethasone, prednisolone, tranilast, cyclosporine, everolimus, mycophenolic acid, sirolimus, tacrolimus, estradiol, and combinations of any of these.

34. The article according to claim 23 wherein total bioactive agent content within the first coated layer is 50% or less.

35. The article according to claim 31 wherein upon placement of the article in a biological environment, the bioactive agent is released, and wherein release is 35% or less within 24 hours after placement of the article in the biological environment.

36. The article according to claim 31 wherein the bioactive agent release is 20% or less within seven days after placement of the article in the biological environment.

37. The article according to claim 31 wherein the bioactive agent is released at a therapeutically effective concentration for at least one week, when the article is implanted in a patient.

38. The article according to claim 23 wherein the coating is provided on a surface of the article that comprises less than 100% of total article surface area.

39. The article according to claim 23 wherein the bioactive agent releasing coating further comprises a coating layer comprising parylene, silane, siloxane, polyurethane, polybutadiene, polycarbodiimide, or a combination of any of these.

40. The article according to claim 23 wherein the surface is provided with surface texture.

41. The article according to claim 23 wherein the article is a stent, graft, catheter, valve, cardiac device, ophthalmic device, or wound dressing.

42. An implantable medical article having a bioactive agent releasing coating at a surface, the coating comprising:
(a) a first coated layer comprising bioactive agent and a first biodegradable polymer, wherein the first biodegradable polymer is a copolymer of polyalkylene glycol terephthalate and an aromatic polyester; and
(b) a second coated layer covering at least a portion of the first coated layer and comprising a second biodegradable polymer, and
(c) a third coated layer comprising a copolymer of polyalkylene glycol terephthalate and an aromatic polyester,
wherein the first biodegradable polymer and the second biodegradable polymer are different,
and wherein the second biodegradable polymer is selected to have a slower bioactive agent release rate relative to the first biodegradable polymer.

43. The article according to claim 42 wherein the copolymer of the third coated layer is the same as the first biodegradable polymer of the first coated layer.

44. A method for preparing an implantable medical article comprising steps of:
(a) providing a medical article;
(b) disposing a first coating composition on a surface of the medical article, the first coating composition comprising bioactive agent and a first biodegradable polymer, wherein the first biodegradable polymer is a copolymer of polyalkylene glycol terephthalate and an aromatic polyester, to provide a first coated layer on the article; and
(c) disposing a second coating composition on the first coated layer, the second coating composition comprising a second biodegradable polymer that is different from the first biodegradable polymer,
wherein the second biodegradable polymer is selected to have a slower bioactive agent release rate relative to the first biodegradable polymer.

45. A method for preparing an implantable medical article comprising steps of:
(a) providing a medical article;
(b) disposing a first coating composition on a surface of the medical article, the first coating composition comprising bioactive agent and a second biodegradable polymer, to provide a first coated layer on the article; and
(c) disposing an outer coating composition on the first coated layer, the outer coating composition comprising a polyetherester copolymer that is a copolymer of polyalkylene glycol terephthalate and an aromatic polyester,
wherein the polyetherester copolymer and the second biodegradable polymer are different,
and wherein the second biodegradable polymer is selected to have a slower bioactive agent release rate relative to the polyetherester copolymer.

46. A method of delivering bioactive agent to a patient in a controlled manner, the method comprising steps of:
(a) providing the device according to claim 1 to a patient, and
(b) maintaining the device in the patient for a selected period of time, during which time the bioactive agent is released from the coating composition in a controlled manner.
